(19) United States
(12) Patent Application Publication
(10) Pub. No.: US 2016/0361401 A1
(43) Pub. Date: Dec. 15, 2016

(54) COMPOSITIONS AND METHODS FOR THE TREATMENT OF HER2/NEU OVER-EXPRESSING TUMORS

(71) Applicants: ADVAXIS, INC., Princeton, NJ (US); THE TRUSTEES OF THE UNIVERSITY OF PENNSYLVANIA, Philadephia, PA (US)

(72) Inventors: Vafa Shahabi, Valley Forge, PA (US); Anu Wallecha, Yardley, PA (US); Paulo C. Maciag, Long Grove, IL (US); Yvonne Paterson, Philadelphia, PA (US); Nicola Mason, Philadelphia, PA (US); Matthew Seavey, Secane, PA (US)

(21) Appl. No.: 15/121,421

(22) PCT Filed: Feb. 25, 2015
(86) PCT No.: PCT/US15/17559

§ 371 Date: Aug. 25, 2016

Related U.S. Application Data
(63) Continuation of application No. 14/189,008, filed on Feb. 25, 2014.
Application No. 14/189,008 is a continuation-in-part of application No. 13/210,696, filed on Aug. 16, 2011, now Pat. No. 9,017,660.
Application No. 13/210,696 is a continuation-in-part of application No. 12/945,386, filed on Nov. 12, 2010, now Pat. No. 9,084,747.
Application No. PCT/US15/17559 is a continuation of application No. 14/268,436, filed on May 2, 2014.
Application No. 14/268,436 is a continuation-in-part of application No. 14/189,008, filed on Feb. 25, 2014.
Application No. 14/189,008 is a continuation-in-part of application No. 13/210,696, filed on Aug. 16, 2011, now Pat. No. 9,017,660.
Application No. 13/210,696 is a continuation-in-part of application No. 12/945,386, filed on Nov. 12, 2010, now Pat. No. 9,084,747.
(60) Provisional application No. 61/260,277, filed on Nov. 11, 2009.
(60) Provisional application No. 61/260,277, filed on Nov. 11, 2009.
(60) Provisional application No. 62/076,411, filed on Nov. 6, 2014.

(51) Int. Cl. A61K 39/00; C07K 14/195; C12N 9/90; C12N 9/12; C12N 1/36; C12N 1/20; C07K 14/82; C12N 15/74

(52) U.S. Cl. CPC A61K 39/0011; C07K 14/82; C07K 14/195; C12N 15/74; C12N 9/12; C12Y 207/10; C12N 1/36; C12N 1/20; C12N 9/90; C12Y 501/01001; A61K 2039/523; A61K 2039/522; A61K 2039/545; A61K 2039/552; A61K 2039/55; A61K 2039/572; C07K 2319/40

(57) ABSTRACT

This invention provides compositions and methods for treating and vaccinating against a HER2/neu antigen-expressing tumor and inducing an immune response against the same in a subject.

### FIELD OF INVENTION

This invention provides compositions and methods for inducing an immune response against a HER2/neu antigen-expressing tumor and for treating the same and vaccinating against the same in human and canine subjects. In another embodiment, a human subject is a child or adolescent.

### BACKGROUND OF THE INVENTION

Listeria monocytogenes is an intracellular pathogen that primarily infects antigen presenting cells and has adapted for life in the cytoplasm of these cells. Host cells, such as macrophages, actively phagocytose L. monocytogenes and the majority of the bacteria are degraded in the phagolysosome. Some of the bacteria escape into the host cytosol by perforating the phagosomal membrane through the action of a hemolysin, listeriolysin O (LLO). Once in the cytosol, L. monocytogenes can polymerize the host actin and pass directly from cell to cell further evading the host immune system and resulting in a negligible antibody response to L. monocytogenes.

HER2/neu (also referred to herein as “Her-2”) is a 185 kDa glycoprotein that is a member of the epidermal growth factor receptor (EGFR) family of tyrosine kinases, and consists of an extracellular domain, a transmembrane domain, and an intracellular domain which is known to be involved in cellular signaling. In humans, the Her2 antigen is overexpressed in 25 to 40% of all breast cancers and is also overexpressed in many cancers of the bone (osteosarcoma—OSA), ovaries, lung, pancreas, brain, and gastrointestinal tract. The overexpression of Her-2 is associated with uncontrolled cell growth and signaling, both of which contribute to the development of tumors. Patients with cancers that overexpress Her-2 exhibit tolerance even with detectable humoral, CD8⁺ T cell, and CD4⁺ T cell responses directed against Her-2.

Large breed dogs spontaneously develop OSA that recapitulates many aspects of pediatric OSA including histologic heterogeneity, aggressive local disease and early metastases. In dogs, OSA can occur in any bone but the limbs account for 75%-85% of all affected bones and where it is called ‘appendicular osteosarcoma’. The remaining OSA affect the axial skeleton comprising maxilla, mandible, spine, cranium, ribs, nasal cavity, paranasal sinuses and pelvis. At diagnosis, 95% of dogs have micrometastatic disease and despite amputation and chemotherapy, the median survival time is 10 months with most dogs euthanized due to progressive metastatic disease. Pulmonary metastatic disease is the principal cause of morbidity and mortality in both species.

Primary malignant bone tumors in the pediatric to young adult populations are relatively uncommon and account for about 6% of all cancers in those less than 20 years old and 3% of all cancers in adolescents and young adults (AYA) within the age range of 15 to 29 years. Osteosarcoma affects about 400 children and teens in the U.S. every year, representing a small, high need area that has seen little therapeutics improvement in decades. Although osteosarcoma (OS) is a rare malignancy, it is ranked among the leading causes of cancer-related death in the pediatric age group. Modern, multiagent, dose-intensive chemotherapy in conjunction with surgery achieves a 5-year event-free survival of 60-70% in extremity localized, non-metastatic disease. However, a major, as yet unsolved, problem is the poor prognosis for metastatic relapse or recurrence, and for patients with axial disease. Moreover, there are no products approved for osteosarcoma in the U.S, presenting a high need for novel therapies that address this disease.

The present invention meets this need by providing a recombinant Listeria-HER2/neu vaccine strain that was generated using the LmddA vaccine vector which has a well-defined attenuation mechanism and is devoid of antibiotic selection markers and which has been found effective in treating canine osteosarcoma.

### SUMMARY OF THE INVENTION

In one aspect, the invention provided herein relates to an immunogenic composition comprising a fusion polypeptide, wherein said fusion polypeptide comprises a HER2/neu chimeric antigen fused to an additional polypeptide, and wherein administering the fusion protein to a subject having a HER2/neu-expressing tumor circumvents mutation avoidance by the tumor. In another embodiment, circumventing mutation avoidance is due to epitope spreading. In yet another embodiment, circumventing mutation avoidance is due to the chimeric nature of the antigen.

In another embodiment, the invention provided herein relates to a recombinant Listeria vaccine strain comprising a nucleic acid molecule, wherein and in another embodiment, the nucleic acid molecule comprises a first open reading frame encoding a polypeptide, wherein the polypeptide comprises a HER2/neu chimeric antigen, wherein the nucleic acid molecule further comprises a second open reading frame encoding a metabolic enzyme, and wherein the metabolic enzyme complements an endogenous gene that is mutated in the chromosome of the recombinant Listeria strain.

In one embodiment, the invention provided herein relates to a method of treating a HER2/neu-expressing tumor growth or cancer in a subject, the method comprising the step of administering a recombinant attenuated Listeria comprising a nucleic acid encoding a fusion polypeptide, wherein said fusion polypeptide comprises a HER2/neu chimeric antigen fused to an additional polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is mutated in the chromosome of said recombinant Listeria vaccine strain. In another embodiment, the subject is a human. In another embodiment, a human subject may be an adult or a child. In another embodiment, a subject is a canine. In another embodiment, the chimeric HER2 is a canine chimeric HER2. In another embodiment, the chimeric HER2 is a human chimeric HER2. In another embodiment, administering said fusion polypeptide to said subject prevents escape mutations within said tumor. In another embodiment, said human HER2/neu chimeric antigen comprises at least 5, 9, 13, 14, or 17 of the mapped human MHC-class I epitopes.

In another embodiment, the invention provided herein relates to a method of preventing a HER2/neu-expressing tumor growth or cancer.

In one embodiment, a method of treating a HER2/neu-expressing tumor growth or cancer, results in increased overall survival of said subject. In another embodiment, a method of treating a HER2/neu-expressing tumor growth or cancer, results in a delay of metastatic disease in a subject. In another embodiment, the treating results in an increased HER2/neu specific T cell response.

In one embodiment, this invention provides a method of eliciting an enhanced immune response against a HER2/neu-expressing tumor growth or cancer in a subject, the method comprising the step of administering a recombinant attenuated Listeria comprising a nucleic acid encoding a fusion polypeptide, wherein said fusion polypeptide comprises a HER2/neu chimeric antigen fused to an additional polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is lacking in the chromosome of said recombinant Listeria vaccine strain. In another embodiment, said method of eliciting an enhanced immune response results in increased overall survival of said subject. In another embodiment, said method of eliciting an enhanced immune response results in a delay of metastatic disease in a subject. In another embodiment, said method of eliciting an enhanced immune response results in an increased HER2/neu specific T cell response.

In one embodiment, this invention provides a method of prolonging survival in a subject suffering a HER2/neu-expressing tumor growth or cancer, the method comprising the step of administering a recombinant attenuated Listeria comprising a nucleic acid encoding a fusion polypeptide, wherein said fusion polypeptide comprises a HER2/neu chimeric antigen fused to an additional polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is lacking in the chromosome of said recombinant Listeria vaccine strain. In another embodiment, the subject is a human. In another embodiment, a human subject may be an adult or a child. In another embodiment, a subject is a canine. In one embodiment, said method further comprises administering said recombinant attenuated Listeria following a relapse or metastasis in said subject.

In one embodiment, the invention provided herein relates to a method of delaying metastatic disease in a subject suffering from a HER2/neu-expressing tumor growth or cancer, the method comprising the step of administering a recombinant attenuated Listeria comprising a nucleic acid encoding a fusion polypeptide, wherein said fusion polypeptide comprises a HER2/neu chimeric antigen fused to an additional polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is lacking in the chromosome of said recombinant Listeria vaccine strain. In another embodiment, a subject in a human. In another embodiment, a human subject may be an adult or a child. In another embodiment, a subject is a canine.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Construction of ADXS31-164. (A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human HER2/neu gene, which was constructed by the direct fusion of 3 fragments the HER2/neu: EC1 (aa 40-170), EC2 (aa 359-518) and ICI (aa 679-808). The vector schematic on the right shows details pAdv164 expressing a chimeric HER2/neu fusion protein consisting of 2 extracellular domains and one intracellular domain of human HER2/neu fused to truncated LLO. The plasmid is maintained within the recombinant dal/dat/actA⁻listeria strain (LmddA) by means of auxotrophic complementation of the dal gene (See Examples). (B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIG. 2. Immunogenic properties of ADXS31-164 (A) Cytotoxic T cell responses elicited by HER2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (B) IFN-γ secreted by the splenocytes from immunized FVB/N mice into the cell culture medium, measured by ELISA, after 24 hours of in vitro stimulation with mitomycin C treated NT-2 cells. (C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. *P value<0.001.

FIG. 3. Tumor Prevention Studies for Listeria-ChHER2/neu Vaccines HER2/neu transgenic mice were injected six times with each recombinant Listeria-ChHer2 or a control Listeria vaccine Immunizations started at 6 weeks of age and continued every three weeks until week 21. Appearance of tumors was monitored on a weekly basis and expressed as percentage of tumor free mice. *p<0.05, N=9 per group.

FIG. 4. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIG. 5. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (A). dot-plots of the Tregs from a representative experiment. (B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIG. 6. Vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. (A) Ex vivo imaging of the mice was performed on the indicated days using a Xenogen X-100 CCD camera. (B) Pixel intensity was graphed as number of photons per second per cm2 of surface area; this is shown as average radiance. (C) Expression of HER2/neu by EMT6-Luc cells, 4T1-Luc and NT-2 cell lines was detected by Western blots, using an anti-HER2/neu antibody. J774.A2 cells, a murine macrophage like cell line was used as a negative control.

FIG. 7. Shows the first 18 patients vaccinated with ADXS31-164.

FIG. 8. Shows that ADXS31-164 administration does not cause early or late cardiac damage. A) Echocardiogram of the heart showing that the heart looks normal. B) Sequential cardiactroponin I levels evaluated over the course of the study showing that the levels are normal (see also FIG. 26D).

FIG. 9. Shows ADXS31-164 associated changes in A) body temperature and B) systolic blood pressure. Body temperature and systolic blood pressure were recorded at baseline and every 2 hours post ADXS31-164 administration. Parameters for each dog at each vaccination are displayed. Horizontal bars represent median values for all dogs in each dose group at each time point. *p<0.05, **p<0.005

FIG. 10. Shows treatment schedule of combination ADXS31-164 and palliative radiation therapy (RT) in primary disease.

FIG. 11. Radiograph showing no evidence of metastatic disease in a dog following fracture of proximal humerus and also shows the presence of honey callus indicating fracture healing.

FIG. 12. Timeline of a pilot phase I clinical trial to evaluate the safety and efficacy of a L. monocytogenes recombinant expressing ADXS31-164 to elicit therapeutically effective anti-tumor immunity in dogs with appendicular osteosarcoma.

FIG. 13. Treatment-related adverse events and survival curves following ADXS-31-164 administration. A) Treatment-related adverse events. B) All dogs without metastatic disease at the time of trial enrollment. Dogs in the control group underwent limb amputation followed by either carboplatin alone or carboplatin plus Adriamycin. 2 dogs have been censored from the vaccine arm as they died of unrelated causes (1 dog died from aspiration pneumonia, the other died from nephroblastoma). Vaccinated group Red line; Control group Black line.

FIG. 14. Radiographic images of primary and metastatic osteosarcoma (OSA) in a human (A) and canine (B) patient. In both species, primary lesions are characterized by areas of marked proliferation and lysis in the bone metaphysis (arrows in A).

FIG. 15. Schematic of the phase I, 3+3 clinical trial to evaluate the safety and efficacy of ADXS31-164 in dogs with HER2+ osteosarcoma (OSA). Privately owned dogs with spontaneous HER2+ appendicular OSA underwent standard of care amputation and follow up carboplatin chemotherapy. Three weeks after the last carboplatin dose, dogs were vaccinated with either 2×10⁸, 5×10⁸, 1×10⁹ or 3×10⁹ CFU of ADXS31-164 intravenously (three vaccinations given three weeks apart). Dogs were re-staged every 2 months until death to determine vaccine efficacy in preventing metastatic disease.

FIG. 16. HER2/neu expression in canine primary osteosarcoma. (A) H&E stain of primary OSA from a dog showing nests of malignant osteoblasts and osteoid deposition. (B) Immunohistochemical evaluation of canine primary OSA showing HER2/neu expression within malignant osteoblasts. (C) Western blot of primary OSA samples from 5 privately owned dogs showing variable expression of HER2/neu. Positive controls are: MCF-7 human mammary carcinoma cell line andCAMAC2 a canine mammary carcinoma cell line.

FIG. 17. Hematological values at baseline and at 24 hours post ADXS31-164 administration. Pre and post values from all dogs within each dose group at each vaccination were averaged. *p<0.05, **p<0.005. Shows a transient, but statistically significant increase in white blood cell and neutrophil counts (A-B) that occurred 24 hours after ADXS31-164 administration and that were accompanied by a transient decrease in platelets and lymphocytes (C-D).

FIG. 18. ADXS31-164 induced increases in white blood cells (WBC), neutrophil and monocyte counts correlate with survival. WBC, neutrophil and monocyte counts were measured at baseline and 24 hours after vaccination. The percent increase was calculated following each vaccination and averaged for each dog. (A) Results are displayed according to survival (dead or alive). (B) Results are displayed according to ADXS31-164 dose received. Horizontal bars represent median values of the group.

FIG. 19. Shows the results of evaluation of Her-2 specific T cell responses induced by ADXS31-164 by IFN-γ ELISpot.

FIG. 20. Shows repeat “booster” vaccinations Stimulate Her-2 specific immunity. (A) Shows the results for patient 289-003. (B) Shows the results for patient 289-004. EC1, EC2 and IC1 represent the peptide fragments of the HER2/neu polypeptide.

FIG. 21. Kaplan Meier estimates for (A) Time To Metastasis (TTM) and (B) OSA Specific Survival.

FIG. 22. Shows that ADXS31-164 prevents development of metastatic disease. (A and B) Thoracic radiographs taken 3 weeks after carboplatin therapy (A) and 3 weeks after the third ADXS31-164 vaccine (B) showing an increase in size of the pre-existing metastatic nodule in the right cranial lung lobe but lack of further metastatic disease development in remaining lung lobes. (C and D) Pulmonary nodule identified on thoracoscopy that fluoresces under near infra-red light following administration of ICG (C). Grossly normal appearing pulmonary tissue removed at the time of metastatectomy showing fluorescence under near infra-red light (inset) (D). (E and F) H&E stained histopathology of (E) pulmonary nodule and (F) fluorescing normal pulmonary tissue showing significant hemorrhage and necrosis of encapsulated pulmonary nodule (E) and focal area of inflammation in grossly normal appearing pulmonary tissue (F). (G and H) Immunohistochemistry of pulmonary nodule at low (G) and high (H) magnification showing CD3+ T cells surrounding the pulmonary nodule with minimal CD3+ T cells within the neoplastic tissue. (I and J) Immunohistochemistry of normal appearing pulmonary tissue at low (G) and high (H) magnification showing focal accumulations of CD3+ T cells. (K) High magnification H&E stain of focal pneumonia showing large abnormal cells with mitotic figures surrounded by lymphocytes. (L) Vimentin stain of pneumonic region showing large cells, with mitotic figures surrounded by mononuclear cells.

FIG. 23. ADXS31-164 delays/prevents metastatic disease and prolongs overall survival in dogs with spontaneous HER2+ osteosarcoma. Shown is a Kaplan-Meier survival curve of vaccinated dogs compared with a historical control group. The control group consisted of dogs with HER2+ appendicular OSA, treated with amputation and follow-up chemotherapy but who did not receive ADXS31-164. P<0.0001. Vaccinated group Red line; Control group Black line.

FIG. 24. Shows that ADXS31-164 breaks tolerance to HER2/neu. PBMCs were collected at baseline, 3 weeks after the 3^{rd} vaccine (9 weeks) and 2 months later (17 weeks) and analyzed by IFN-γ ELISpot for responses to the highly conserved IC1 domain of HER2/neu. Results presented divided dogs into early responders, late responders and apparent non-responders. NA indicates that the 17 week sample for these dogs was not yet evaluated.

FIG. 25A-D. Shows that ADXS31-164 does not adversely affect cardiac function. Cardiac parameters LVID (diastole) (FIG. 25A), LVID (systole) (FIG. 25B) and fractional shortening (FIG. 25C) were evaluated for each dog at baseline, the time of vaccination and every 2 months thereafter. Cardiac troponin I levels were evaluated at the same time points (FIG. 25D).

FIG. 26. Shows that ADXS31-164 breaks immune tolerance to the highly conserved intracellular domain of HER2/neu.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, provided herein are compositions and methods for preventing, treating and vaccinating against a Her2-neu antigen-expressing tumor and inducing an immune response against sub-dominant epitopes of the Her2-neu antigen, while circumventing mutation avoidance. In another embodiment, circumventing mutation avoidance is due to epitope spreading. In yet another embodiment, circumventing mutation avoidance is due to the chimeric nature of the antigen.

In another embodiment, provided herein is an immunogenic composition comprising a fusion polypeptide, wherein said fusion polypeptide comprises a HER2/neu chimeric antigen fused to an additional polypeptide, and wherein administering the fusion protein to a subject having an HER2/neu-expressing tumor prevents escape mutations within said tumor. In another embodiment, provided herein is a recombinant Listeria vaccine strain comprising the immunogenic composition.

In one embodiment, a subject is a human subject. In another embodiment, the human subject is an adult or a child. In another embodiment, the human subject is a child. In another embodiment, a subject is a canine subject. In another embodiment, the canine is a dog.

In one embodiment, provided herein is a method of eliciting an enhanced immune response against a HER2/neu-expressing tumor growth or cancer in a subject, the method comprising the step of administering a recombinant Listeria comprising a nucleic acid encoding a fusion polypeptide, wherein said fusion polypeptide comprises a HER2/neu chimeric antigen fused to an additional polypeptide.

In one embodiment, provided herein is a method of preventing a HER2/neu-expressing tumor growth or cancer in a subject, the method comprising the step of administering a recombinant Listeria comprising nucleic acid encoding a fusion polypeptide, wherein said fusion polypeptide comprises a HER2/neu chimeric antigen fused to an additional polypeptide.

In another embodiment, provided herein is a method of treating a HER2/neu-expressing tumor growth or cancer in a subject, the method comprising the step of administering a recombinant Listeria comprising nucleic acid encoding a fusion polypeptide, wherein said fusion polypeptide comprises a HER2/neu chimeric antigen fused to an additional polypeptide.

In one embodiment, provided herein is a method of prolonging the survival of a subject having a HER2/neu-expressing tumor growth or cancer, the method comprising the step of administering a recombinant Listeria comprising nucleic acid encoding a fusion polypeptide, wherein said fusion polypeptide comprises a HER2/neu chimeric antigen fused to an additional polypeptide. In one embodiment, the subject is a human. In another embodiment, the subject is a canine.

In one embodiment, provided herein is a method of delaying metastatic disease in a subject having a HER2/neu-expressing tumor growth or cancer, the method comprising the step of administering a recombinant Listeria comprising nucleic acid encoding a fusion polypeptide, wherein said fusion polypeptide comprises a HER2/neu chimeric antigen fused to an additional polypeptide. In one embodiment, the subject is a human. In another embodiment, the subject is a canine.

In one embodiment, provided herein is a method of treating a HER2/neu-expressing tumor growth or cancer in a subject, the method comprising the step of administering a recombinant attenuated Listeria comprising a nucleic acid encoding a fusion polypeptide, wherein said fusion polypeptide comprises a HER2/neu chimeric antigen fused to an additional polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is mutated in the chromosome of said recombinant Listeria vaccine strain. In another embodiment, the subject is a human. In another embodiment, a human subject may be an adult or a child. In another embodiment, a subject is a canine. In another embodiment, the chimeric HER2 is a canine chimeric HER2. In another embodiment, the chimeric HER2 is a human chimeric HER2. In another embodiment, administering said fusion polypeptide to said subject prevents escape mutations within said tumor. In another embodiment, said human HER2/neu chimeric antigen comprises at least 5, 9, 13, 14, or 17 of the mapped human MHC-class I epitopes.

In one embodiment, provided herein is a recombinant Listeria vaccine strain comprising a nucleic acid molecule, wherein the nucleic acid molecule comprises a first open reading frame encoding a polypeptide, wherein the polypeptide comprises a HER2/neu chimeric antigen, wherein the nucleic acid molecule further comprises a second open reading frame encoding a metabolic enzyme, and wherein the metabolic enzyme complements an endogenous gene that is lacking in the chromosome of the recombinant Listeria strain. In another embodiment, the recombinant Listeria vaccine strain further comprises a nucleic acid molecule comprising a third open reading frame encoding a metabolic enzyme, and wherein the metabolic enzyme complements an endogenous gene that is lacking in the chromosome of the recombinant Listeria strain.

In one embodiment, the nucleic acid molecule is integrated into the Listeria genome. In another embodiment, the nucleic acid molecule is in a plasmid in the recombinant Listeria vaccine strain. In yet another embodiment, the plasmid is stably maintained in the recombinant Listeria vaccine strain in the absence of antibiotic selection. In another embodiment, the plasmid does not confer antibiotic resistance upon the recombinant Listeria. In another embodiment, the recombinant Listeria strain is attenuated. In another embodiment, the recombinant Listeria is an attenuated auxotrophic strain. In another embodiment, the high metabolic burden that the expression of a foreign antigen exerts on a bacterium such as one of the present invention is also an important mechanism of attenuation.

In one embodiment the attenuated strain is LmddA. In another embodiment, this strain exerts a strong adjuvant effect which is an inherent property of Listeria-based vaccines. One manifestation of this adjuvant effect is the 5-fold decrease in the number of the intratumoral Tregs caused by either Listeria expressing an antigen other than chimeric HER2/neu or the ADXS-31-164 (expressing chimeric HER2/neu) vaccines (see FIG. 5 herein). In another embodiment, the LmddA vector expressing a different antigen (HPV 16 E7) is also associated with a significant decrease in the frequency of Tregs in the tumors, likely as a consequence of innate immunity responses.

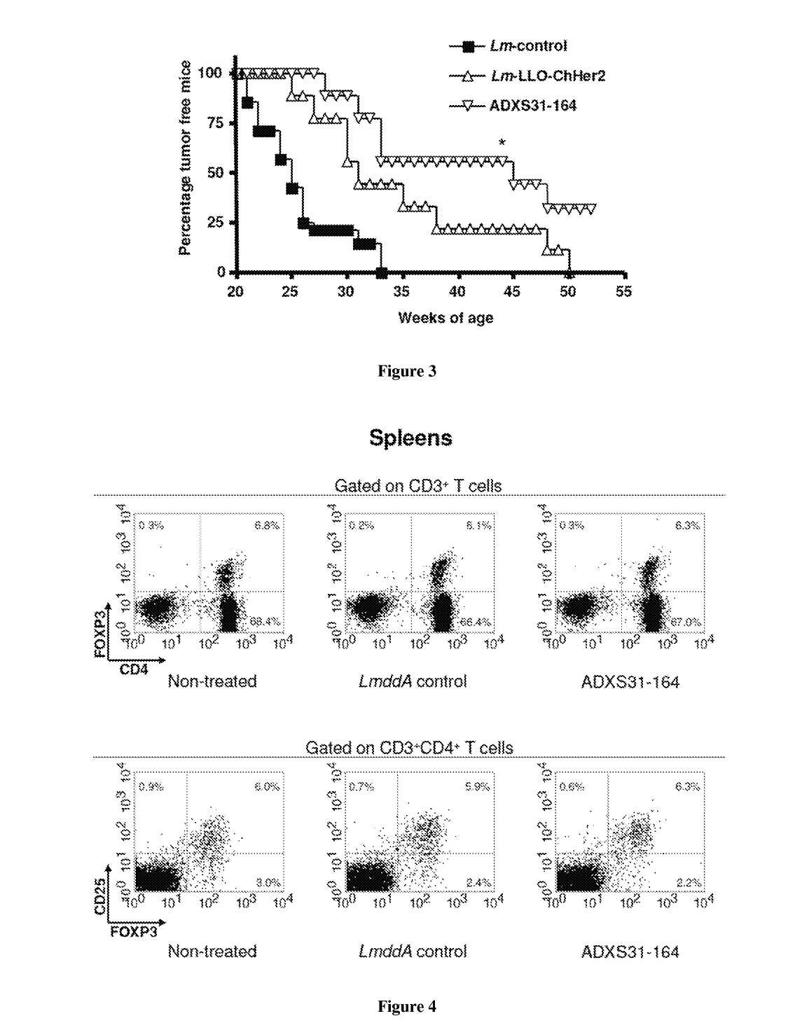

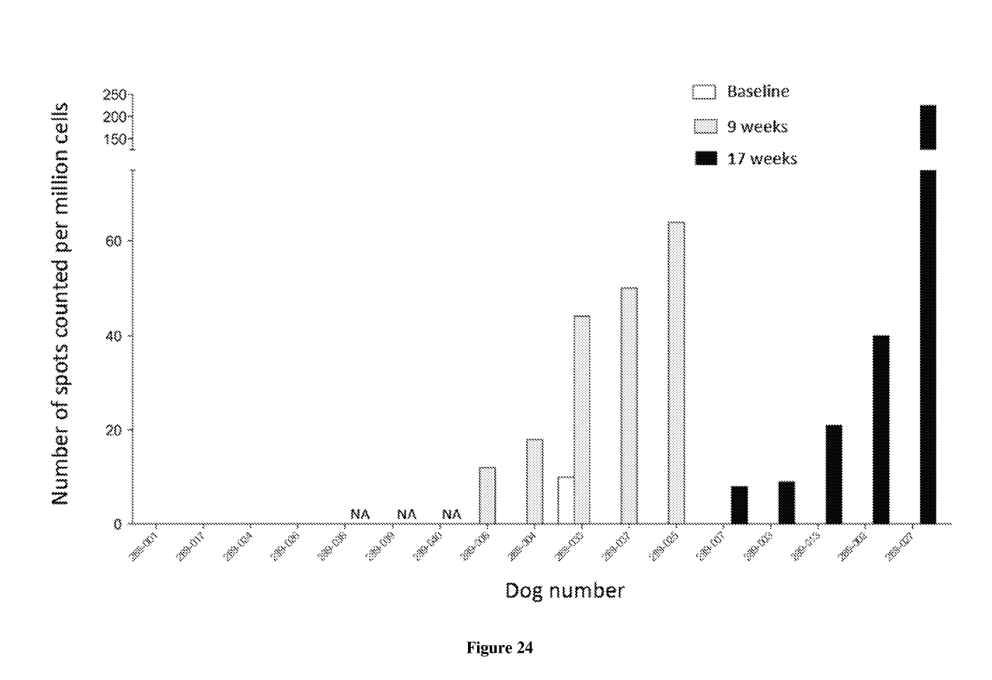

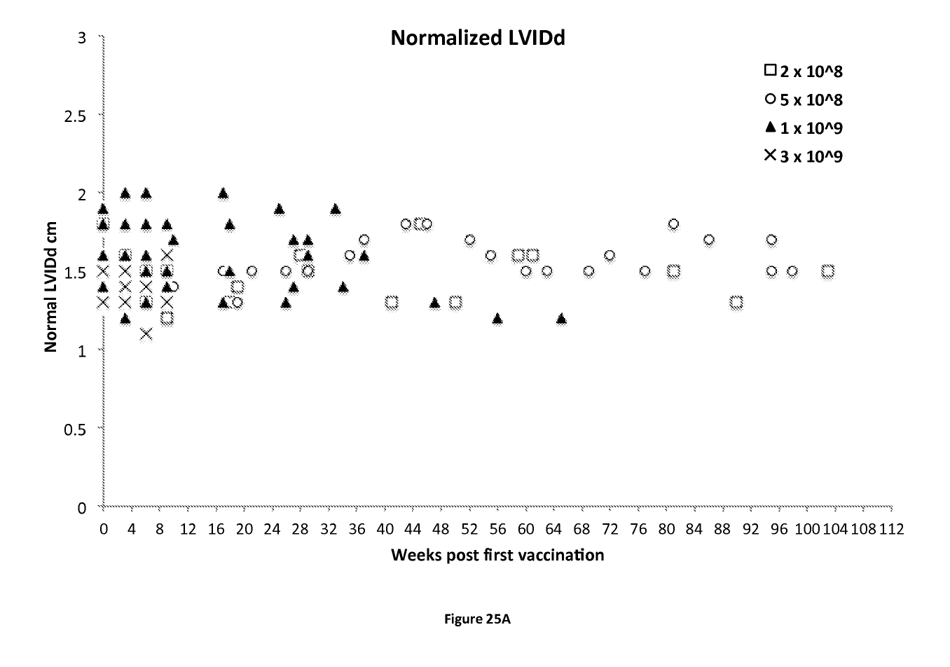

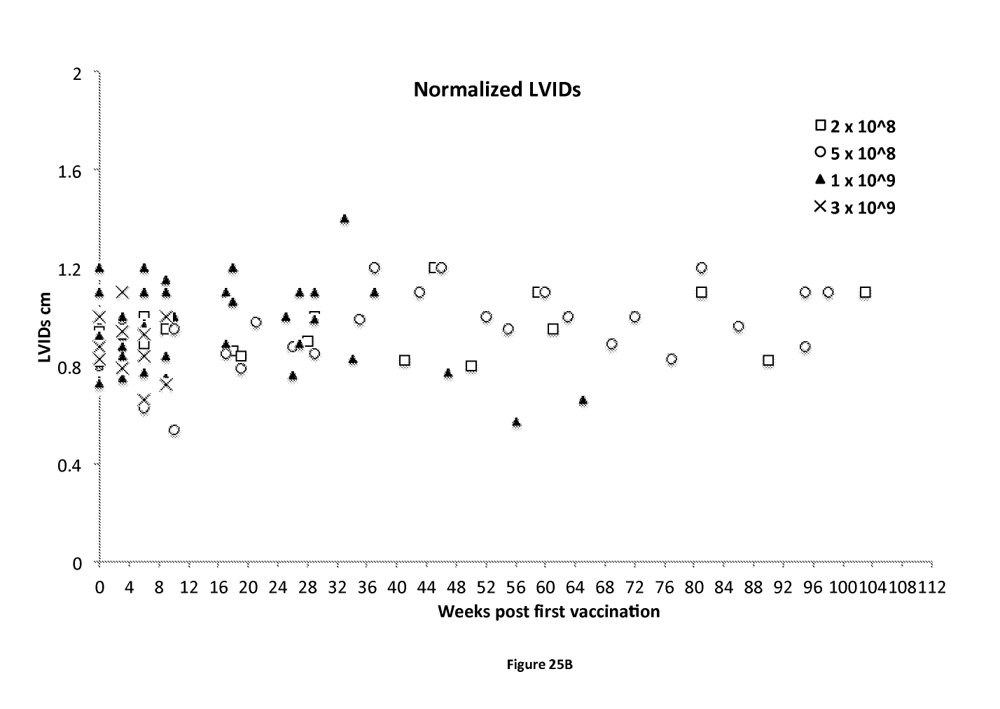

In one embodiment, an attenuated auxotrophic Listeria vaccine strain provided herein is the ADXS-31-164 strain. ADXS-31-164 is based on a Listeria vaccine vector which is attenuated due to the deletion of virulence gene actA and retains the plasmid for HER2/neu expression in vivo and in vitro by complementation of dal gene. In one embodiment, ADXS31-164 expresses and secretes a chimeric HER2/neu protein fused to the first 441 amino acids of listeriolysin 0 (LLO), which in another embodiment, is a truncated and non-hemolytic LLO. In another embodiment, ADXS31-164 exerts strong and antigen specific anti-tumor responses with ability to break tolerance toward HER2/neu in transgenic animals (see Examples, FIG. 24). In another embodiment, the ADXS31-164 strain is highly attenuated and has a better safety profile than previous Listeria vaccine generation, as it is more rapidly cleared from the spleens of the immunized mice. In another embodiment, the ADXS31-164 results in a longer delay of tumor onset in transgenic animals than Lm-LLO-ChHer2, the antibiotic resistant and more virulent version of this vaccine (see FIG. 3). In another embodiment, ADXS31-164 strain is highly immunogenic, able to break tolerance toward the HER2/neu self-antigen and prevent tumor formation in HER2/neu transgenic animals. In another embodiment, ADXS31-164 causes a significant decrease in intra-tumoral T regulatory cells (Tregs). In another embodiment, the lower frequency of Tregs in tumors treated with LmddA vaccines resulted in an increased intratumoral CD8/Tregs ratio, suggesting that a more favorable tumor microenvironment can be obtained after immunization with LmddA vaccines. In another embodiment, the use of this chimeric antigen does not result in escape mutations indicating that tumors do not mutate away from a therapeutic efficacious response to treatment with this novel antigen (see Example 6). In another embodiment, peripheral immunization with ADXS31-164 delays the growth of a metastatic breast cancer cell line in the brain (see Example 7). In another embodiment, canine subjects suffering from osteosarcoma and provided treatment including amputation, chemotherapy, and vaccination with ADXS31-164, have prolonged survival compared with control subjects not receiving the vaccination with ADXS31-164. (See Examples 9 and 10). In another embodiment, canine subjects suffering from osteosarcoma and provided treatment including amputation, chemotherapy, and vaccination with ADXS31-164, show reduced metastasis compared with control subjects not receiving the vaccination with ADXS31-164. (See Example 10). In another embodiment, canine subjects suffering from osteosarcoma and provided treatment including amputation, chemotherapy, and vaccination with ADXS31-164, show increased specific T cell response induced compared with control subjects not receiving the vaccination with ADXS31-164. (See Example 10).

In one embodiment, the Lm-LLO-ChHer2 strain is Lm-LLO-138, and comprises an antibiotic resistance gene and a prfA gene expressed from a plasmid.

In one embodiment, recombinant attenuated, antibiotic-free Listeria-expressing chimeric antigens are useful for preventing, and treating a cancer or solid tumors, as exemplified herein. In another embodiment, the tumor is a HER2/neu positive tumor. In another embodiment, the cancer is a HER2/neu-expressing cancer. In another embodiment, the cancer is breast cancer, a central nervous system (CNS) cancer, a head and neck cancer, an osteosarcoma (OSA), a canine osteosarcoma, Ewing's sarcoma (ES), or any HER2/neu-expressing cancer known in the art. In another embodiment, a canine osteosarcoma is an appendicular osteosarcoma. In another embodiment, the tumor is an osteosarcoma tumor, a breast tumor, a head and neck tumor, or any other antigen-expressing tumor known in the art. In another embodiment, a cancer or solid tumor described herein is a result of relapse or metastatic disease.

In another embodiment, recombinant Listeria expressing a chimeric HER2/neu are useful as a therapeutic vaccine for the treatment of HER2/neu overexpressing solid tumors. In another embodiment, a HER2/neu chimeric antigen provided herein is useful for treating HER2/neu-expressing tumors and preventing escape mutations of the same. In another embodiment, the term “escape mutation” refers to a tumor mutating away from a therapeutic efficacious response to treatment.

In one embodiment, provided herein is a nucleic acid molecule comprising a first open reading frame encoding a recombinant polypeptide provided herein, wherein the nucleic molecule resides within the recombinant Listeria vaccine strain. In another embodiment, the nucleic acid molecule provided herein is used to transform the Listeria in order to arrive at a recombinant Listeria. In another embodiment, the nucleic acid provided herein lacks a virulence gene. In another embodiment, the nucleic acid molecule integrated into the Listeria genome carries a non-functional virulence gene. In another embodiment, the virulence gene is mutated in the genome of the recombinant Listeria. In yet another embodiment, the nucleic acid molecule is used to inactivate the endogenous gene present in the Listeria genome. In yet another embodiment, the virulence gene is an actA gene. In another embodiment, the virulence gene is a prfA gene. As will be understood by a skilled artisan, the virulence gene can be any gene known in the art to be associated with virulence in the recombinant Listeria.

In one embodiment, a metabolic gene, a virulence gene, etc., provided herein is lacking in a chromosome of the Listeria strain. In another embodiment, the metabolic gene, virulence gene, etc., provided herein is lacking in the chromosome and in any episomal genetic element of the Listeria strain. It will be appreciated by a skilled artisan that the term “episome,” “episomal,” etc., refer to a plasmid vector or use thereof that does not integrate into the chromosome of the Listeria provided herein. In another embodiment, the term refers to plasmid vectors that integrate into the chromosome of the Listeria provided herein. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In another embodiment, the nucleic acids and plasmids provided herein do not confer antibiotic resistance upon a recombinant Listeria provided herein.

In one embodiment, a nucleic acid molecule provided herein comprises a plasmid. In another embodiment, a nucleic acid molecule provided herein is a plasmid. In another embodiment, a plasmid provided herein is an integration vector. In another embodiment, a plasmid is a non-integration vector. In another embodiment, a plasmid comprises an integration vector. In another embodiment, an integration vector is a site-specific integration vector. In another embodiment, a nucleic acid molecule of methods and compositions of the present invention are composed of any type of nucleotide known in the art.

It will be understood by a skilled artisan that the term “metabolic enzyme” may encompass an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the present invention.

It will be understood by a skilled artisan that the term “Stably maintained” may encompass maintenance of a nucleic acid molecule or plasmid in a host cell or bacteria in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In one embodiment, the present invention provides a recombinant Listeria strain expressing the antigen. The present invention also provides recombinant polypeptides comprising a listeriolysin (LLO) protein fragment fused to a HER2 chimeric protein or fragment thereof, vaccines and immunogenic compositions comprising same, and methods of inducing an anti-HER2 immune response and treating and vaccinating against a HER2-expressing tumor, comprising the same.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed by any other method known in the art.

In one embodiment, a recombinant polypeptide provided herein comprises a fusion protein provided herein. In another embodiment, the recombinant polypeptide is a fusion protein. In another embodiment, a fusion protein provided herein comprises a chimeric HER2 antigen and an additional polypeptide selected from the group consisting of: a) non-hemolytic LLO protein or N-terminal fragment, b) a PEST sequence, or c) an ActA fragment, and further wherein said additional polypeptide is fused to the HER2/neu chimeric antigen. In another embodiment, the additional polypeptide is functional. In another embodiment, a fragment of the additional polypeptide is immunogenic. In another embodiment, the additional polypeptide is immunogenic.

In another embodiment, a fusion protein provided herein comprises a non-hemolytic LLO protein or N-terminal fragment fused to a HER2/neu chimeric antigen provided herein. In another embodiment, a fusion protein of methods and compositions of the present invention comprises an ActA sequence from a Listeria organism. ActA proteins and fragments thereof augment antigen presentation and immunity in a similar fashion to LLO.

In another embodiment, a fusion protein of methods and compositions of the present invention comprises a truncated ActA-sequence from a Listeria organism. In another embodiment, a truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the nonconservative QDNKR substitution as described in US Patent Publication Serial No. 2014/0186387. ActA proteins and fragments thereof augment antigen presentation and immunity in a similar fashion to LLO.

In another embodiment of methods and compositions of the present invention, a fusion protein provided herein comprises the HER2/neu antigen and an additional polypeptide. In one embodiment, the additional polypeptide is a non-hemolytic LLO protein or fragment thereof (Examples herein). In another embodiment, the additional polypeptide is a PEST sequence. In another embodiment, the additional polypeptide is an ActA protein or a fragment thereof. ActA proteins and fragments thereof augment antigen presentation and immunity in a similar fashion to LLO.

The additional polypeptide of methods and compositions of the present invention is, in another embodiment, a listeriolysin (LLO) peptide. In another embodiment, the additional polypeptide is an ActA peptide. In another embodiment, the additional polypeptide is a PEST sequence peptide. In another embodiment, the additional polypeptide is any other peptide capable of enhancing the immunogenicity of an antigen peptide. Each possibility represents a separate embodiment of the present invention.

Fusion proteins comprising the HER2/neu chimeric antigen may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen provided herein can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). In another embodiment, the antigen is ligated into a plasmid. Each method represents a separate embodiment of the present invention.

The results of the present invention demonstrate that administration of a composition of the present invention has utility for inducing formation of antigen-specific T cells (e.g. cytotoxic T cells) that recognize and kill tumor cells (Examples herein).

In one embodiment, the present invention provides a recombinant polypeptide comprising an N-terminal fragment of an LLO protein fused to a HER2 chimeric protein or fused to a fragment thereof. In one embodiment, the present invention provides a recombinant polypeptide consisting of an N-terminal fragment of an LLO protein fused to a HER2 chimeric protein or fused to a fragment thereof.

In another embodiment, a HER2 chimeric protein of the methods and compositions of the present invention is a human HER2 chimeric protein. In another embodiment, the HER2 chimeric protein is a mouse HER2 chimeric protein. In another embodiment, the HER2 chimeric protein is a rat HER2 chimeric protein. In another embodiment, the HER2 chimeric protein is a primate HER2 chimeric protein. In another embodiment, the HER2 chimeric protein is a canine HER2 chimeric protein. In another embodiment, the Her-2 protein is a HER2 chimeric protein of human or any other animal species or combinations thereof known in the art. Each possibility represents a separate embodiment of the present invention.

In another embodiment, a Her-2 protein is a protein referred to as “HER2/neu,” “Erbb2,” “v-erb-b2,” “c-erb-b2,” “neu,” or “cNeu.” In another embodiment, HER2/neu is also referred to herein as “Her-2,” “Her-2 protein,” “HER2 protein,” or “HER2”).

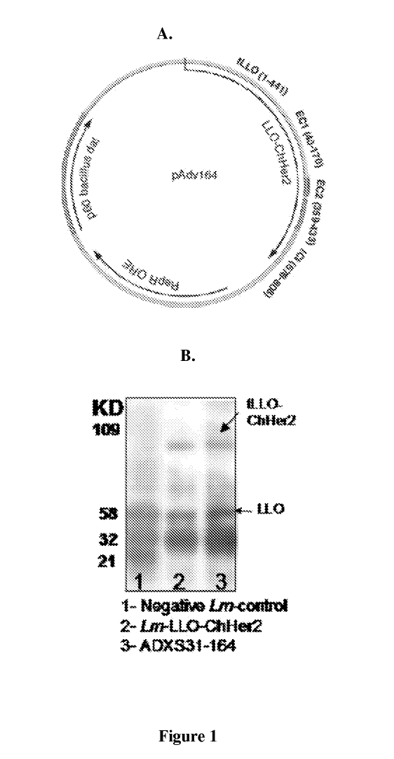

In one embodiment, a Her2-neu chimeric protein provided herein harbors two of the extracellular and one intracellular fragments of HER2/neu antigen showing clusters of MHC-class I epitopes of the oncogene, where, in another embodiment, the chimeric protein, harbors 3 H2Dq and at least 17 of the mapped human MHC-class I epitopes of the HER2/neu antigen (fragments EC1, EC2, and IC1) (See FIG. 1A). In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2). In another embodiment, the Her2-neu chimeric protein is fused to a non-hemolytic listeriolysin O (LLO). In another embodiment, the Her2-neu chimeric protein is fused to truncated listeriolysin O (tLLO). In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the expression and secretion of the fusion protein tLLO-ChHer2 from the attenuated auxotrophic strain provided herein that expresses a chimeric HER2/neu antigen/LLO fusion protein is comparable to that of the Lm-LLO-ChHer2 in TCA precipitated cell culture supernatants after 8 hours of in vitro growth (See FIG. 1B).

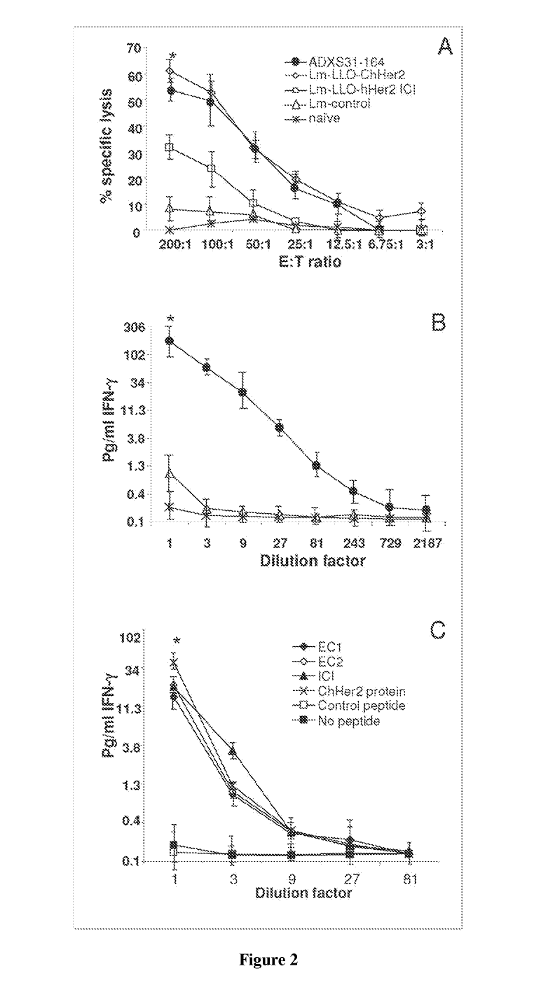

In one embodiment, no CTL activity is detected in naïve animals or mice injected with an irrelevant Listeria vaccine (See FIG. 2A). While in another embodiment, the attenuated auxotrophic strain (ADXS31-164) provided herein is able to stimulate the secretion of IFN-γ by the splenocytes from wild type FVB/N mice (FIG. 2B).

In another embodiment, the metabolic enzyme of the methods and compositions provided herein is an amino acid metabolism enzyme, where, in another embodiment, the metabolic enzyme is an alanine racemase enzyme. In another embodiment, the metabolic enzyme is a D-amino acid transferase enzyme. In another embodiment, the metabolic enzyme catalyzes a formation of an amino acid used for a cell wall synthesis in the recombinant Listeria strain, where in another embodiment, the metabolic enzyme is an alanine racemase enzyme.

In another embodiment, the gene encoding the metabolic enzyme is expressed under the control of the Listeria p60 promoter. In another embodiment, the inlA (encodes internalin) promoter is used. In another embodiment, the hly promoter is used. In another embodiment, the ActA promoter is used. In another embodiment, the integrase gene is expressed under the control of any other gram positive promoter. In another embodiment, the gene encoding the metabolic enzyme is expressed under the control of any other promoter that functions in Listeria. The skilled artisan will appreciate that other promoters or polycistronic expression cassettes may be used to drive the expression of the gene. Each possibility represents a separate embodiment of the present invention.

In another embodiment, a HER2/neu chimeric protein is encoded by the following nucleic acid sequence set forth in SEQ ID NO:1 gagacccacctggacatgctccgccacctctaccagggctgccaggtggtgcagggaaacctggaactcacctacctgcccacca atgccagcctgtccttcctgcaggatatccaggaggtgcagggctacgtgctcatcgctcacaaccaagtgaggcaggtcccactgcag aggctgcggattgtgcgaggcacccagctctttgaggacaactatgccctggccgtgctagacaatggagacccgctgaacaataccac ccctgtcacaggggcctccccaggaggcctgcgggagctgcagcttcgaagcctcacagagatcttgaaaggaggggtcttgatccag cggaacccccagctctgctaccaggac acgattttgtggaagaatatccaggagtttgctggctgc aagaagatctttgggagcctggc a tttctgccggagagctttgatggggacccagcctccaacactgccccgctccagcc agagcagctccaagtgtttgagactctggaaga gatcacaggttacctatacatctcagcatggccggacagcctgcctgacctcagcgtcttccagaacctgcaagtaatccggggacgaat tctgcacaatggcgcctactcgctgaccctgcaagggctgggc atcagctggctggggctgcgctcactgagggaactgggcagtgga ctggccctcatccaccataacacccacctctgcttcgtgcacacggtgccctgggaccagctctttcggaacccgcaccaagctctgctc cacactgccaaccggccagaggacgagtgtgtgggcgagggcctggcctgccaccagctgtgcgcccgagggcagcagaagatcc ggaagtacacgatgcggagactgctgcaggaaacggagctggtggagccgctgacacctagcggagcgatgcccaaccaggcgc gatgcggatcctgaaagagacggagctgaggaaggtgaaggtgcttggatctggcgcttttggcacagtctacaagggcatctggatcc ctgatggggagaatgtgaaaattccagtggccatcaaagtgttgagggaaaacacatcccccaaagccaacaaagaaatcttagacgaa gcatacgtgatggctggtgtgggctccccatatgtctcccgccttctgggcatctgcctgacatccacggtgcagctggtgacac agctta tgccctatggctgcctcttagactaa (SEQ ID NO: 1).

In another embodiment, the HER2/neu chimeric protein comprises the sequence of SEQ ID NO: 2:

(SEQ ID NO: 2)

E T H L D M L R H L Y Q G C Q V V Q G N L E L T Y
L P T N A S L S F L Q D I Q E V Q G Y V L I A H N
Q V R Q V P L Q R L R I V R G T Q L F E D N Y A L
A V L D N G D P L N N T T P V T G A S P G G L R E
L Q L R S L T E I L K G G V L I Q R N P Q L C Y Q
D T I L W K N I Q E F A G C K K I F G S L A F L P
E S F D G D P A S N T A P L Q P E Q L Q V F E T L
E E I T G Y L Y I S A W P D S L P D L S V F Q N L
Q V I R G R I L H N G A Y S L T L Q G L G I S W L
G L R S L R E L G S G L A L I H H N T H L C F V H
T V P W D Q L F R N P H Q A L L H T A N R P E D E
C V G E G L A C H Q L C A R G Q Q K I R K Y T M R
R L L Q E T E L V E P L T P S G A M P N Q A Q M R
I L K E T E L R K V K V L G S G A F G T V Y K G I
W I P D G E N V K I P V A I K V L R E N T S P K A
N K E I L D E A Y V M A G V G S P Y V S R L L G I
C L T S T V Q L V T Q L M P Y G C L L D.

Table 1 below shows the percent (%) identity between the amino acid sequences of human and canine Her-2 EC and IC fragments, respectively.

TABLE 1
89% identity EC1

| | | |
|---|---|---|
| Human | SLSFLQDIQDVQGYVLIAHHQVRQVPLQRLRIVRGTQLPEDNYALAVLDNGDPLHHTTPV | 60 |
| Canine | SLSPLQDIQDVQGYVLIAHSQVRQIPLQRLRIVRGTQLPEDNYALAVLDNGDPLEGGIPA | 60 |
| | *******************.****:*****************************:. *. | |
| Human | TGASPGGLRELQLRSLTDILKGGVLIQRNPQLCYQDTILWKOIPHKNNQLALTLIDTHRE | 120 |
| Canine | PGAAPGGLRELQLRSLTDILKGGVLIQRSPQLCHQDTILWKDVPHKNNQLALTLIDTNRS | 120 |
| | .**:************************.****:********:***************** | |
| Human | RACHPCSPHCK 131 SEQ ID NO: 69 | |
| Canine | RACPPCSPACK 131 SEQ ID NO: 70 | |
| | *** **** ** | |

93% identity EC2

| | | |
|---|---|---|
| Human | TAPLQPEQLQVPDTLDDITGYLYISAKPDSLPDLSVTQNLQVIRGRILHSGAYSLTLQGL | 60 |
| Canine | TAPLQPEQLRVPEALEEITGYLYISAWPDSLPNLSVTQNLRVIRGRVLHDGAYSLTLQGL | 60 |
| | *********:***:******************:*******:*****:**:********** | |
| Human | GISHLGLRSLRRLGS 75 SEQ ID NO: 71 | |
| Canine | GISHLGLRSLHELGS 75 SEQ ID NO: 72 | |
| | *************** | |
| Human | NQAQNRILMDTELRKVKVLGSGATGTVYKGIWIPDGDNVKIPVAIKVLRKNTSPKANKSI | 60 |
| Canine | NQAQMRILKDTELRKVKVLGSGATGTVYKGIWIPDGENVKIPVAIKVLRDNTSPKANKSI | 60 |
| | ************************************************************ | |

98% identity IC1

| | | |
|---|---|---|
| Human | LDEAYVMACVGSPYVSRLLGICLTSTVQLVTQLMPYGCLLDHVRKKRGRLGSQDLLHWCH | 120 |
| Canine | LDEAYVHAGVGSPYVSRLLGICLTSTVQLVTQLMPYCCLLDHVRDHRCRLGSQDLLNWCV | 120 |
| | *********************************************:*************: | |
| Human | QIAKCMSYLED 131 SEQ ID NO: 73 | |
| Canine | QIAKCMSYLED 131 SEQ ID NO: 74 | |
| | *********** | |

In another embodiment, an amino acid sequence encoding a human HER2/EC1 fragment is set forth in (SEQ ID NO: ₆₉):

(SEQ ID NO: 69)

SLSFLQDIQEVQGYVLIAHNQVRQVPLQRLRIVRGTQLFEDNYALAVLDN
GDPLNNTTPVTGASPGGLRELQLRSLTEILKGGVLIQRNPQLCYQDTILW
KDIFHKNNQLALTLIDTNRSRACHPCSPMCK.

In another embodiment, an amino acid sequence encoding a canine her2/neu EC1 fragment is set forth in (SEQ ID NO: 70):

(SEQ ID NO: 70)

SLSFLQDIQEVQGYVLIAHSQVRQIPLQRLRIVRGTQLFEDNYALAVLDN
GDPLEGGIPAPGAAPGGLRELQLRSLTEILKGGVLIQRSPQLCHQDTILW
KDVFHKNNQLALTLIDTNRSRACPPCSPACK.

In another embodiment, an amino acid sequence encoding a human her2/neu EC2 fragment is set forth in (SEQ ID NO: 71):

(SEQ ID NO: 71)

TAPLQPEQLQVFETLEEITGYLYISAWPDSLPDLSVFQNLQVIRGRILHN
GAYSLTLQGLGISWLGLRSLRELGS.

In another embodiment, an amino acid sequence encoding a canine her2/neu EC2 fragment is set forth in (SEQ ID NO: 72):

(SEQ ID NO: 72)

TAPLQPEQLRVFEALEEITGYLYISAWPDSLPNLSVFQNLRVIRGRVLHD
GAYSLTLQGLGISWLGLRSLRELGS.

In another embodiment, an amino acid sequence encoding a human her2/neu IC1 fragment is set forth in (SEQ ID NO: 73):

(SEQ ID NO: 73)

NQAQMRILKETELRKVKVLGSGAFGTVYKGIWIPDGENVKIPVAIKVLRE
NTSPKANKEILDEAYVMAGVGSPYVSRLLGICLTSTVQLVTQLMPYGCLL
DHVRENRGRLGSQDLLNWCMQIAKGMSYLED.

In another embodiment, an amino acid sequence encoding a canine her2/neu IC1 fragment is set forth in (SEQ ID NO: 74):

(SEQ ID NO: 74)

NQAQMRILKETELRKVKVLGSGAFGTVYKGIWIPDGENVKIPVAIKVLRE
NTSPKANKEILDEAYVMAGVGSPYVSRLLGICLTSTVQLVTQLMPYGCLL
DHVRENRGRLGSQDLLNWCMQIAKGMSYLED.

In one embodiment, the human amino acid sequence of HER2 EC1 fragment (SEQ ID NO: 69) has 89% identity with that of a canine HER2 EC1 fragment (SEQ ID NO: 70). In another embodiment, the human amino acid sequence of HER2 EC2 fragment (SEQ ID NO: 71) has 93% identity with that of a canine HER2 EC2 fragment (SEQ ID NO: 72). In another embodiment, the human amino acid sequence of HER2 IC1 fragment (SEQ ID NO: 73) has 98% identity with that of a canine HER2 IC1 fragment (SEQ ID NO: 74).

In one embodiment, the HER2 chimeric protein or fragment thereof of the methods and compositions provided herein does not include a signal sequence thereof. In another embodiment, omission of the signal sequence enables the HER2 fragment to be successfully expressed in Listeria, due the high hydrophobicity of the signal sequence. Each possibility represents a separate embodiment of the present invention.

In another embodiment, the fragment of a HER2 chimeric protein of methods and compositions of the present invention does not include a transmembrane domain (TM) thereof. In one embodiment, omission of the TM enables the HER2 fragment to be successfully expressed in Listeria, due the high hydrophobicity of the TM. Each possibility represents a separate embodiment of the present invention.

In one embodiment, the nucleic acid sequence encoding a rat-HER2/neu gene is comprises SEQ ID NO: 45:

(SEQ ID NO: 45)

CCGGAATCGCGGGCACCCAAGTGTGTACCGGCACAGACATGAAGTTGCGG
CTCCCTGCCAGTCCTGAGACCCACCTGGACATGCTCCGCCACCTGTACCA
GGGCTGTCAGGTAGTGCAGGGCAACTTGGAGCTTACCTACGTGCCTGCCA
ATGCCAGCCTCTCATTCCTGCAGGACATCCAGGAAGTTCAGGGTTACATG
CTCATCGCTCACAACCAGGTGAAGCGCGTCCCACTGCAAAGGCTGCGCAT
CGTGAGAGGGACCCAGCTCTTTGAGGACAAGTATGCCCTGGCTGTGCTAG
ACAACCGAGATCCTCAGGACAATGTCGCCGCCTCCACCCCAGGCAGAACC
CCAGAGGGGCTGCGGGAGCTGCAGCTTCGAAGTCTCACAGAGATCCTGAA
GGGAGGAGTTTTGATCCGTGGGAACCCTCAGCTCTGCTACCAGGACATGG
TTTTGTGGAAGGACGTCTTCCGCAAGAATAACCAACTGGCTCCTGTCGAT
ATAGACACCAATCGTTCCCGGGCCTGTCCACCTTGTGCCCCCGCCTGCAA
AGACAATCACTGTTGGGGTGAGAGTCCGGAAGACTGTCAGATCTTGACTG
GCACCATCTGTACCAGTGGTTGTGCCCGGTGCAAGGGCCGGCTGCCCACT
GACTGCTGCCATGAGCAGTGTGCCGCAGGCTGCACGGGCCCCAAGCATTC
TGACTGCCTGGCCTGCCTCCACTTCAATCATAGTGGTATCTGTGAGCTGC
ACTGCCCAGCCCTCGTCACCTACAACACAGACACCTTTGAGTCCATGCAC
AACCCTGAGGGTCGCTACACCTTTGGTGCCAGCTGCGTGACCACCTGCCC
CTACAACTACCTGTCTACGGAAGTGGGATCCTGCACTCTGGTGTGTCCCC
CGAATAACCAAGAGGTCACAGCTGAGGACGGAACACAGCGTTGTGAGAAA
TGCAGCAAGCCCTGTGCTCGAGTGTGCTATGGTCTGGGCATGGAGCACCT
TCGAGGGGCGAGGGCCATCACCAGTGACAATGTCCAGGAGTTTGATGGCT
GCAAGAAGATCTTTGGGAGCCTGGCATTTTTGCCGGAGAGCTTTGATGGG
GACCCCTCCTCCGGCATTGCTCCGCTGAGGCCTGAGCAGCTCCAAGTGTT
CGAAACCCTGGAGGAGATCACAGGTTACCTGTACATCTCAGCATGGCCAG
ACAGTCTCCGTGACCTCAGTGTCTTCCAGAACCTTCGAATCATTCGGGGA
CGGATTCTCCACGATGGCGCGTACTCATTGACACTGCAAGGCCTGGGGAT
CCACTCGCTGGGGCTGCGCTCACTGCGGGAGCTGGGCAGTGGATTGGCTC
TGATTCACCGCAACGCCCATCTCTGCTTTGTACACACTGTACCTTGGGAC
CAGCTCTTCCGGAACCCACATCAGGCCCTGCTCCACAGTGGGAACCGGCC
GGAAGAGGATTGTGGTCTCGAGGGCTTGGTCTGTAACTCACTGTGTGCCC
ACGGGCACTGCTGGGGGCCAGGGCCCACCCAGTGTGTCAACTGCAGTCAT
TTCCTTCGGGGCCAGGAGTGTGTGGAGGAGTGCCGAGTATGGAAGGGGCT
CCCCCGGGAGTATGTGAGTGACAAGCGCTGTCTGCCGTGTCACCCCGAGT
GTCAGCCTCAAAACAGCTCAGAGACCTGCTTTGGATCGGAGGCTGATCAG
TGTGCAGCCTGCGCCCACTACAAGGACTCGTCCTCCTGTGTGGCTCGCTG
CCCCAGTGGTGTGAAACCGGACCTCTCCTACATGCCCATCTGGAAGTACC
CGGATGAGGAGGGCATATGCCAGCCGTGCCCCATCAACTGCACCCACTCC
TGTGTGGATCTGGATGAACGAGGCTGCCCAGCAGAGCAGAGAGCCAGCCC
GGTGACATTCATCATTGCAACTGTAGTGGGCGTCCTGCTGTTCCTGATCT
TAGTGGTGGTCGTTGGAATCCTAATCAAACGAAGGAGACAGAAGATCCGG
AAGTATACGATGCGTAGGCTGCTGCAGGAAACTGAGTTAGTGGAGCCGCT
GACGCCCAGCGGAGCAATGCCCAACCAGGCTCAGATGCGGATCCTAAAAG
AGACGGAGCTAAGGAAGGTGAAGGTGCTTGGATCAGGAGCTTTTGGCACT
GTCTACAAGGGCATCTGGATCCCAGATGGGGAGAATGTGAAAATCCCCGT
GGCTATCAAGGTGTTGAGAGAAAACACATCTCCTAAAGCCAACAAAGAAA
TTCTAGATGAAGCGTATGTGATGGCTGGTGTGGGTTCTCCGTATGTGTCC
CGCCTCCTGGGCATCTGCCTGACATCCACAGTACAGCTGGTGACACAGCT
TATGCCCTACGGCTGCCTTCTGGACCATGTCCGAGAACACCGAGGTCGCC
TAGGCTCCCAGGACCTGCTCAACTGGTGTGTTCAGATTGCCAAGGGGATG
AGCTACCTGGAGGACGTGCGGCTTGTACACAGGGACCTGGCTGCCCGGAA
TGTGCTAGTCAAGAGTCCCAACCACGTCAAGATTACAGATTTCGGGCTGG
CTCGGCTGCTGGACATTGATGAGACAGAGTACCATGCAGATGGGGGCAAG
GTGCCCATCAAATGGATGGCATTGGAATCTATTCTCAGACGCCGGTTCAC
CCATCAGAGTGATGTGTGGAGCTATGGAGTGACTGTGTGGGAGCTGATGA
CTTTTGGGGCCAAACCTTACGATGGAATCCCAGCCCGGGAGATCCCTGAT
TTGCTGGAGAAGGGAGAACGCCTACCTCAGCCTCCAATCTGCACCATTGA
TGTCTACATGATTATGGTCAAATGTTGGATGATTGACTCTGAATGTCGCC
CGAGATTCCGGGAGTTGGTGTCAGAATTTTCACGTATGGCGAGGGACCCC
CAGCGTTTTGTGGTCATCCAGAACGAGGACTTGGGCCCATCCAGCCCCAT
GGACAGTACCTTCTACCGTTCACTGCTGGAAGATGATGACATGGGTGACC
TGGTAGACGCTGAAGAGTATCTGGTGCCCCAGCAGGGATTCTTCTCCCCG
GACCCTACCCCAGGCACTGGGAGCACAGCCCATAGAAGGCACCGCAGCTC
GTCCACCAGGAGTGGAGGTGGTGAGCTGACACTGGGCCTGGAGCCCTCGG
AAGAAGGGCCCCCCAGATCTCCACTGGCTCCCTCGGAAGGGGCTGGCTCC
GATGTGTTTGATGGTGACCTGGCAATGGGGGTAACCAAAGGGCTGCAGAG
CCTCTCTCCACATGACCTCAGCCCTCTACAGCGGTACAGCGAGGACCCCA
CATTACCTCTGCCCCCCGAGACTGATGGCTATGTTGCTCCCCTGGCCTGC
AGCCCCCAGCCCGAGTATGTGAACCAATCAGAGGTTCAGCCTCAGCCTCC
TTTAACCCCAGAGGGTCCTCTGCCTCCTGTCCGGCCTGCTGGTGCTACTC
TAGAAAGACCCAAGACTCTCTCTCCTGGGAAGAATGGGGTTGTCAAAGAC
GTTTTTGCCTTCGGGGGTGCTGTGGAGAACCCTGAATACTTAGTACCGAG
AGAAGGCACTGCCTCTCCGCCCCACCCTTCTCCTGCCTTCAGCCCAGCCT
TTGACAACCTCTATTACTGGGACCAGAACTCATCGGAGCAGGGGCCTCCA
CCAAGTAACTTTGAAGGGACCCCCACTGCAGAGAACCCTGAGTACCTAGG
CCTGGATGTACCTGTA.

In one embodiment, the nucleic acid sequence encoding a rat/HER2/neu EC1 fragment comprises SEQ ID NO: 46:

(SEQ ID NO: 46)

CCCAGGCAGAACCCCAGAGGGGCTGCGGGAGCTGCAGCTTCGAAGTCTCA
CAGAGATCCTGAAGGGAGGAGTTTTGATCCGTGGGAACCCTCAGCTCTGC
TACCAGGACATGGTTTTGTGGAAGGACGTCTTCCGCAAGAATAACCAACT
GGCTCCTGTCGATATAGACACCAATCGTTCCCGGGCCTGTCCACCTTGTG
CCCCCGCCTGCAAAGACAATCACTGTTGGGGTGAGAGTCCGGAAGACTGT
CAGATCTTGACTGGCACCATCTGTACCAGTGGTTGTGCCCGGTGCAAGGG
CCGGCTGCCCACTGACTGCTGCCATGAGCAGTGTGCCGCAGGCTGCACGG
GCCCCAAGCA.

In another embodiment, the nucleic acid sequence encoding the rat HER2/neu EC2 fragment comprises SEQ ID NO: 47:

(SEQ ID NO: 47)

GGTCACAGCTGAGGACGGAACACAGCGTTGTGAGAAATGCAGCAAGCCCT
GTGCTCGAGTGTGCTATGGTCTGGGCATGGAGCACCTTCGAGGGGCGAGG
GCCATCACCAGTGACAATGTCCAGGAGTTTGATGGCTGCAAGAAGATCTT
TGGGAGCCTGGCATTTTTGCCGGAGAGCTTTGATGGGGACCCCTCCTCCG
GCATTGCTCCGCTGAGGCCTGAGCAGCTCCAAGTGTTCGAAACCCTGGAG
GAGATCACAGGTTACCTGTACATCTCAGCATGGCCAGACAGTCTCCGTGA
CCTCAGTGTCTTCCAGAACCTTCGAATCATTCGGGGACGGATTCTCCACG
ATGGCGCGTACTCATTGACACTGCAAGGCCTGGGGATCCACTCGCTGGGG
CTGCGCTCACTGCGGGAGCTGGGCAGTGGATTGGCTCTGATTCACCGCAA
CGCCCATCTCTGCTTTGTACACACTGTACCTTGGGACCAGCTCTTCCGGA
ACCCACATCAGGCCCTGCTCCACAGTGGGAACCGGCCGGAAGAGGATTGT
GGTCTCGAGGGCTTGGTCTGTAACTCACTGTGTGCCCACGGGCACTGCTG
GGGGCCAGGGCCCACCCA.

In another embodiment, the nucleic acid sequence encoding the rat HER2/neu IC1 fragment comprises SEQ ID NO: 48:

(SEQ ID NO: 48)

CGCCCAGCGGAGCAATGCCCAACCAGGCTCAGATGCGGATCCTAAAAGAG
ACGGAGCTAAGGAAGGTGAAGGTGCTTGGATCAGGAGCTTTTGGCACTGT
CTACAAGGGCATCTGGATCCCAGATGGGGAGAATGTGAAAATCCCCGTGG
CTATCAAGGTGTTGAGAGAAAACACATCTCCTAAAGCCAACAAAGAAATT
CTAGATGAAGCGTATGTGATGGCTGGTGTGGGTTCTCCGTATGTGTCCCG
CCTCCTGGGCATCTGCCTGACATCCACAGTACAGCTGGTGACACAGCTTA
TGCCCTACGGCTGCCTTCTGGACCATGTCCGAGAACACCGAGGTCGCCTA
GGCTCCCAGGACCTGCTCAACTGGTGTGTTCAGATTGCCAAGGGGATGAG
CTACCTGGAGGACGTGCGGCTTGTACACAGGGACCTGGCTGCCCGGAATG
TGCTAGTCAAGAGTCCCAACCACGTCAAGATTACAGATTTCGGGCTGGCT
CGGCTGCTGGACATTGATGAGACAGAGTACCATGCAGATGGGGGCAAGGT
GCCCATCAAATGGATGGCATTGGAATCTATTCTCAGACGCCGGTTCACCC
ATCAGAGTGATGTGTGGAGCTATGGAGTGACTGTGTGGGAGCTGATGACT
TTTGGGGCCAAACCTTACGATGGAATCCCAGCCCGGGAGATCCCTGATTT
GCTGGAGAAGGGAGAACGCCTACCTCAGCCTCCAATCTGCACCATTGATG
TCTACATGATTATGGTCAAATGTTGGATGATTGACTCTGAATGTCGCCCG
AGATTCCGGGAGTTGGTGTCAGAATTTTCACGTATGGCGAGGGACCCCCA
GCGTTTTGTGGTCATCCAGAACGAGGACTTGGGCCCATCCAGCCCCATGG
ACAGTACCTTCTACCGTTCACTGCTGGAA.

In one embodiment, the nucleic acid sequence of human-HER2/neu gene comprises SEQ ID NO: 49:

(SEQ ID NO: 49)

ATGGAGCTGGCGGCCTTGTGCCGCTGGGGGCTCCTCCTCGCCCTCTTGCC
CCCCGGAGCCGCGAGCACCCAAGTGTGCACCGGCACAGACATGAAGCTGC
GGCTCCCTGCCAGTCCCGAGACCCACCTGGACATGCTCCGCCACCTCTAC
CAGGGCTGCCAGGTGGTGCAGGGAAACCTGGAACTCACCTACCTGCCCAC
CAATGCCAGCCTGTCCTTCCTGCAGGATATCCAGGAGGTGCAGGGCTACG
TGCTCATCGCTCACAACCAAGTGAGGCAGGTCCCACTGCAGAGGCTGCGG
ATTGTGCGAGGCACCCAGCTCTTTGAGGACAACTATGCCCTGGCCGTGCT
AGACAATGGAGACCCGCTGAACAATACCACCCCTGTCACAGGGGCCTCCC
CAGGAGGCCTGCGGGAGCTGCAGCTTCGAAGCCTCACAGAGATCTTGAAA
GGAGGGGTCTTGATCCAGCGGAACCCCCAGCTCTGCTACCAGGACACGAT
TTTGTGGAAGGACATCTTCCACAAGAACAACCAGCTGGCTCTCACACTGA
TAGACACCAACCGCTCTCGGGCCTGCCACCCCTGTTCTCCGATGTGTAAG
GGCTCCCGCTGCTGGGGAGAGAGTTCTGAGGATTGTCAGAGCCTGACGCG
CACTGTCTGTGCCGGTGGCTGTGCCCGCTGCAAGGGGCCACTGCCCACTG
ACTGCTGCCATGAGCAGTGTGCTGCCGGCTGCACGGGCCCCAAGCACTCT
GACTGCCTGGCCTGCCTCCACTTCAACCACAGTGGCATCTGTGAGCTGCA
CTGCCCAGCCCTGGTCACCTACAACACAGACACGTTTGAGTCCATGCCCA
ATCCCGAGGGCCGGTATACATTCGGCGCCAGCTGTGTGACTGCCTGTCCC
TACAACTACCTTTCTACGGACGTGGGATCCTGCACCCTCGTCTGCCCCCT
GCACAACCAAGAGGTGACAGCAGAGGATGGAACACAGCGGTGTGAGAAGT
GCAGCAAGCCCTGTGCCCGAGTGTGCTATGGTCTGGGCATGGAGCACTTG
CGAGAGGTGAGGGCAGTTACCAGTGCCAATATCCAGGAGTTTGCTGGCTG
CAAGAAGATCTTTGGGAGCCTGGCATTTCTGCCGGAGAGCTTTGATGGGG
ACCCAGCCTCCAACACTGCCCCGCTCCAGCCAGAGCAGCTCCAAGTGTTT
GAGACTCTGGAAGAGATCACAGGTTACCTATACATCTCAGCATGGCCGGA
CAGCCTGCCTGACCTCAGCGTCTTCCAGAACCTGCAAGTAATCCGGGGAC
GAATTCTGCACAATGGCGCCTACTCGCTGACCCTGCAAGGGCTGGGCATC
AGCTGGCTGGGGCTGCGCTCACTGAGGGAACTGGGCAGTGGACTGGCCCT
CATCCACCATAACACCCACCTCTGCTTCGTGCACACGGTGCCCTGGGACC
AGCTCTTTCGGAACCCGCACCAAGCTCTGCTCCACACTGCCAACCGGCCA
GAGGACGAGTGTGTGGGCGAGGGCCTGGCCTGCCACCAGCTGTGCGCCCG
AGGGCACTGCTGGGGTCCAGGGCCCACCCAGTGTGTCAACTGCAGCCAGT
TCCTTCGGGGCCAGGAGTGCGTGGAGGAATGCCGAGTACTGCAGGGGCTC
CCCAGGGAGTATGTGAATGCCAGGCACTGTTTGCCGTGCCACCCTGAGTG
TCAGCCCCAGAATGGCTCAGTGACCTGTTTTGGACCGGAGGCTGACCAGT
GTGTGGCCTGTGCCCACTATAAGGACCCTCCCTTCTGCGTGGCCCGCTGC
CCCAGCGGTGTGAAACCTGACCTCTCCTACATGCCCATCTGGAAGTTTCC
AGATGAGGAGGGCGCATGCCAGCCTTGCCCCATCAACTGCACCCACTCCT
GTGTGGACCTGGATGACAAGGGCTGCCCCGCCGAGCAGAGAGCCAGCCCT
CTGACGTCCATCGTCTCTGCGGTGGTTGGCATTCTGCTGGTCGTGGTCTT
GGGGGTGGTCTTTGGGATCCTCATCAAGCGACGGCAGCAGAAGATCCGGA
AGTACACGATGCGGAGACTGCTGCAGGAAACGGAGCTGGTGGAGCCGCTG
ACACCTAGCGGAGCGATGCCCAACCAGGCGCAGATGCGGATCCTGAAAGA
GACGGAGCTGAGGAAGGTGAAGGTGCTTGGATCTGGCGCTTTTGGCACAG
TCTACAAGGGCATCTGGATCCCTGATGGGGAGAATGTGAAAATTCCAGTG
GCCATCAAAGTGTTGAGGGAAAACACATCCCCCAAAGCCAACAAAGAAAT
CTTAGACGAAGCATACGTGATGGCTGGTGTGGGCTCCCCATATGTCTCCC
GCCTTCTGGGCATCTGCCTGACATCCACGGTGCAGCTGGTGACACAGCTT
ATGCCCTATGGCTGCCTCTTAGACCATGTCCGGGAAAACCGCGGACGCCT
GGGCTCCCAGGACCTGCTGAACTGGTGTATGCAGATTGCCAAGGGGATGA
GCTACCTGGAGGATGTGCGGCTCGTACACAGGGACTTGGCCGCTCGGAAC
GTGCTGGTCAAGAGTCCCAACCATGTCAAAATTACAGACTTCGGGCTGGC
TCGGCTGCTGGACATTGACGAGACAGAGTACCATGCAGATGGGGGCAAGG
TGCCCATCAAGTGGATGGCGCTGGAGTCCATTCTCCGCCGGCGGTTCACC
CACCAGAGTGATGTGTGGAGTTATGGTGTGACTGTGTGGGAGCTGATGAC
TTTTGGGGCCAAACCTTACGATGGGATCCCAGCCCGGGAGATCCCTGACC
TGCTGGAAAAGGGGGAGCGGCTGCCCCAGCCCCCCATCTGCACCATTGAT
GTCTACATGATCATGGTCAAATGTTGGATGATTGACTCTGAATGTCGGCC
AAGATTCCGGGAGTTGGTGTCTGAATTCTCCCGCATGGCCAGGGACCCCC
AGCGCTTTGTGGTCATCCAGAATGAGGACTTGGGCCCAGCCAGTCCCTTG
GACAGCACCTTCTACCGCTCACTGCTGGAGGACGATGACATGGGGGACCT
GGTGGATGCTGAGGAGTATCTGGTACCCCAGCAGGGCTTCTTCTGTCCAG
ACCCTGCCCCGGGCGCTGGGGGCATGGTCCACCACAGGCACCGCAGCTCA
TCTACCAGGAGTGGCGGTGGGGACCTGACACTAGGGCTGGAGCCCTCTGA
AGAGGAGGCCCCCAGGTCTCCACTGGCACCCTCCGAAGGGGCTGGCTCCG
ATGTATTTGATGGTGACCTGGGAATGGGGGCAGCCAAGGGGCTGCAAAGC
CTCCCCACACATGACCCCAGCCCTCTACAGCGGTACAGTGAGGACCCCAC
AGTACCCCTGCCCTCTGAGACTGATGGCTACGTTGCCCCCCTGACCTGCA
GCCCCCAGCCTGAATATGTGAACCAGCCAGATGTTCGGCCCCAGCCCCCT
TCGCCCCGAGAGGGCCCTCTGCCTGCTGCCCGACCTGCTGGTGCCACTCT
GGAAAGGGCCAAGACTCTCTCCCCAGGGAAGAATGGGGTCGTCAAAGACG
TTTTTGCCTTTGGGGGTGCCGTGGAGAACCCCGAGTACTTGACACCCCAG
GGAGGAGCTGCCCCTCAGCCCCACCCTCCTCCTGCCTTCAGCCCAGCCTT
CGACAACCTCTATTACTGGGACCAGGACCCACCAGAGCGGGGGGCTCCAC
CCAGCACCTTCAAAGGGACACCTACGGCAGAGAACCCAGAGTACCTGGGT
CTGGACGTGCCAGTGTGAACCAGAAGGCCAAGTCCGCAGAAGCCCTGA.

In another embodiment, the nucleic acid sequence encoding a human HER2/neu EC1 fragment implemented into the chimera spans from 120-510 by of the human EC1 region and comprises SEQ ID NO: 50:

(SEQ ID NO: 50)

GAGACCCACCTGGACATGCTCCGCCACCTCTACCAGGGCTGCCAGGTGGT
GCAGGGAAACCTGGAACTCACCTACCTGCCCACCAATGCCAGCCTGTCCT
TCCTGCAGGATATCCAGGAGGTGCAGGGCTACGTGCTCATCGCTCACAAC
CAAGTGAGGCAGGTCCCACTGCAGAGGCTGCGGATTGTGCGAGGCACCCA
GCTCTTTGAGGACAACTATGCCCTGGCCGTGCTAGACAATGGAGACCCGC
TGAACAATACCACCCCTGTCACAGGGGCCTCCCCAGGAGGCCTGCGGGAG
CTGCAGCTTCGAAGCCTCACAGAGATCTTGAAAGGAGGGGTCTTGATCCA
GCGGAACCCCCAGCTCTGCTACCAGGACACGATTTTGTGGAAG.

In one embodiment, the complete EC1 human HER2/neu fragment spans from (58-979 by of the human HER2/neu gene) and is encoded by a nucleic acid sequence comprising SEQ ID NO: 54:

(SEQ ID NO: 54)

GCCGCGAGCACCCAAGTGTGCACCGGCACAGACATGAAGCTGCGGCTCCC
TGCCAGTCCCGAGACCCACCTGGACATGCTCCGCCACCTCTACCAGGGCT
GCCAGGTGGTGCAGGGAAACCTGGAACTCACCTACCTGCCCACCAATGCC
AGCCTGTCCTTCCTGCAGGATATCCAGGAGGTGCAGGGCTACGTGCTCAT
CGCTCACAACCAAGTGAGGCAGGTCCCACTGCAGAGGCTGCGGATTGTGC
GAGGCACCCAGCTCTTTGAGGACAACTATGCCCTGGCCGTGCTAGACAAT
GGAGACCCGCTGAACAATACCACCCCTGTCACAGGGGCCTCCCCAGGAGG
CCTGCGGGAGCTGCAGCTTCGAAGCCTCACAGAGATCTTGAAAGGAGGGG
TCTTGATCCAGCGGAACCCCCAGCTCTGCTACCAGGACACGATTTTGTGG
AAGGACATCTTCCACAAGAACAACCAGCTGGCTCTCACACTGATAGACAC
CAACCGCTCTCGGGCCTGCCACCCCTGTTCTCCGATGTGTAAGGGCTCCC
GCTGCTGGGGAGAGAGTTCTGAGGATTGTCAGAGCCTGACGCGCACTGTC
TGTGCCGGTGGCTGTGCCCGCTGCAAGGGGCCACTGCCCACTGACTGCTG
CCATGAGCAGTGTGCTGCCGGCTGCACGGGCCCCAAGCACTCTGACTGCC
TGGCCTGCCTCCACTTCAACCACAGTGGCATCTGTGAGCTGCACTGCCCA
GCCCTGGTCACCTACAACACAGACACGTTTGAGTCCATGCCCAATCCCGA
GGGCCGGTATACATTCGGCGCCAGCTGTGTGACTGCCTGTCCCTACAACT
ACCTTTCTACGGACGTGGGATCCTGCACCCTCGTCTGCCCCCTGCACAAC
CAAGAGGTGACAGCAGAGGAT.

In another embodiment, the nucleic acid sequence encoding the human HER2/neu EC2 fragment implemented into the chimera spans from 1077-1554 by of the human HER2/neu EC2 fragment and includes a 50 by extension, and comprises SEQ ID NO: 51:

(SEQ ID NO: 51)

AATATCCAGGAGTTTGCTGGCTGCAAGAAGATCTTTGGGAGCCTGGCATT
TCTGCCGGAGAGCTTTGATGGGGACCCAGCCTCCAACACTGCCCCGCTCC
AGCCAGAGCAGCTCCAAGTGTTTGAGACTCTGGAAGAGATCACAGGTTAC
CTATACATCTCAGCATGGCCGGACAGCCTGCCTGACCTCAGCGTCTTCCA
GAACCTGCAAGTAATCCGGGGACGAATTCTGCACAATGGCGCCTACTCGC
TGACCCTGCAAGGGCTGGGCATCAGCTGGCTGGGGCTGCGCTCACTGAGG
GAACTGGGCAGTGGACTGGCCCTCATCCACCATAACACCCACCTCTGCTT
CGTGCACACGGTGCCCTGGGACCAGCTCTTTCGGAACCCGCACCAAGCTC
TGCTCCACACTGCCAACCGGCCAGAGGACGAGTGTGTGGGCGAGGGCCTG
GCCTGCCACCAGCTGTGCGCCCGAGGG.

In one embodiment, a complete EC2 human HER2/neu fragment spans from 907-1504 by of the human HER2/neu gene and is encoded by a nucleic acid sequence comprising SEQ ID NO: 55):

(SEQ ID NO: 55)

TACCTTTCTACGGACGTGGGATCCTGCACCCTCGTCTGCCCCCTGCACAA
CCAAGAGGTGACAGCAGAGGATGGAACACAGCGGTGTGAGAAGTGCAGCA
AGCCCTGTGCCCGAGTGTGCTATGGTCTGGGCATGGAGCACTTGCGAGAG
GTGAGGGCAGTTACCAGTGCCAATATCCAGGAGTTTGCTGGCTGCAAGAA
GATCTTTGGGAGCCTGGCATTTCTGCCGGAGAGCTTTGATGGGGACCCAG
CCTCCAACACTGCCCCGCTCCAGCCAGAGCAGCTCCAAGTGTTTGAGACT
CTGGAAGAGATCACAGGTTACCTATACATCTCAGCATGGCCGGACAGCCT
GCCTGACCTCAGCGTCTTCCAGAACCTGCAAGTAATCCGGGGACGAATTC
TGCACAATGGCGCCTACTCGCTGACCCTGCAAGGGCTGGGCATCAGCTGG
CTGGGGCTGCGCTCACTGAGGGAACTGGGCAGTGGACTGGCCCTCATCCA
CCATAACACCCACCTCTGCTTCGTGCACACGGTGCCCTGGGACCAGCTCT
TTCGGAACCCGCACCAAGCTCTGCTCCACACTGCCAACCGGCCAGAG.

In another embodiment, the nucleic acid sequence encoding the human HER2/neu IC1 fragment implemented into the chimera comprises SEQ ID NO: 52:

(SEQ ID NO: 52)

CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACT.

In another embodiment, the nucleic acid sequence encoding the complete human HER2/neu IC1 fragment spans from 2034-3243 of the human HER2/neu gene and comprises SEQ ID NO: 56):

(SEQ ID NO: 56)

CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACCATGTCCGGG
AAAACCGCGGACGCCTGGGCTCCCAGGACCTGCTGAACTGGTGTATGCAG
ATTGCCAAGGGGATGAGCTACCTGGAGGATGTGCGGCTCGTACACAGGGA
CTTGGCCGCTCGGAACGTGCTGGTCAAGAGTCCCAACCATGTCAAAATTA
CAGACTTCGGGCTGGCTCGGCTGCTGGACATTGACGAGACAGAGTACCAT
GCAGATGGGGGCAAGGTGCCCATCAAGTGGATGGCGCTGGAGTCCATTCT
CCGCCGGCGGTTCACCCACCAGAGTGATGTGTGGAGTTATGGTGTGACTG
TGTGGGAGCTGATGACTTTTGGGGCCAAACCTTACGATGGGATCCCAGCC
CGGGAGATCCCTGACCTGCTGGAAAAGGGGGAGCGGCTGCCCCAGCCCCC
CATCTGCACCATTGATGTCTACATGATCATGGTCAAATGTTGGATGATTG
ACTCTGAATGTCGGCCAAGATTCCGGGAGTTGGTGTCTGAATTCTCCCGC
ATGGCCAGGGACCCCCAGCGCTTTGTGGTCATCCAGAATGAGGACTTGGG
CCCAGCCAGTCCCTTGGACAGCACCTTCTACCGCTCACTGCTGGAGGACG
ATGACATGGGGGACCTGGTGGATGCTGAGGAGTATCTGGTACCCCAGCAG
GGCTTCTTCTGTCCAGACCCTGCCCCGGGCGCTGGGGGCATGGTCCACCA
CAGGCACCGCAGCTCATCTACCAGGAGTGGCGGTGGGGACCTGACACTAG
GGCTGGAGCCCTCTGAAGAGGAGGCCCCCAGGTCTCCACTGGCACCCTCC
GAAGGGGCT.

The LLO utilized in the methods and compositions provided herein is, in one embodiment, a Listeria LLO. In one embodiment, the Listeria from which the LLO is derived is Listeria monocytogenes (LM). In another embodiment, the Listeria is Listeria ivanovii. In another embodiment, the Listeria is Listeria welshimeri. In another embodiment, the Listeria is Listeria seeligeri. In another embodiment, the LLO protein is a non-Listerial LLO protein. In another embodiment, the LLO protein is a synthetic LLO protein. In another embodiment it is a recombinant LLO protein.

In one embodiment, the LLO protein is encoded by a nucleic acid sequence comprising SEQ ID NO: 3:

(SEQ ID NO: 3)

atgaaaaaaataatgctagtttttattacacttatattagttagtctacc
aattgcgcaacaaactgaagcaaaggatgcatctgcattcaataaagaaa
attcaatttcatccatggcaccaccagcatctccgcctgcaagtcctaag
acgccaatcgaaaagaaacacgcggatgaaatcgataagtatatacaagg
attggattacaataaaaacaatgtattagtataccacggagatgcagtga
caaatgtgccgccaagaaaaggttacaaagatggaaatgaatatattgtt
gtggagaaaaagaagaaatccatcaatcaaaataatgcagacattcaagt
tgtgaatgcaatttcgagcctaacctatccaggtgctctcgtaaaagcga
attcggaattagtagaaaatcaaccagatgttctccctgtaaaacgtgat
tcattaacactcagcattgatttgccaggtatgactaatcaagacaataa
aatagttgtaaaaaatgccactaaatcaaacgttaacaacgcagtaaata
cattagtggaaagatggaatgaaaaatatgctcaagcttatccaaatgta
agtgcaaaaattgattatgatgacgaaatggcttacagtgaatcacaatt
aattgcgaaatttggtacagcatttaaagctgtaaataatagcttgaatg
taaacttcggcgcaatcagtgaagggaaaatgcaagaagaagtcattagt
tttaaacaaatttactataacgtgaatgttaatgaacctacaagaccttc
cagatttttcggcaaagctgttactaaagagcagttgcaagcgcttggag
tgaatgcagaaaatcctcctgcatatatctcaagtgtggcgtatggccgt
caagtttatttgaaattatcaactaattcccatagtactaaagtaaaagc
tgcttttgatgctgccgtaagcggaaaatctgtctcaggtgatgtagaac
taacaaatatcatcaaaaattcttccttcaaagccgtaatttacggaggt
tccgcaaaagatgaagttcaaatcatcgacggcaacctcggagacttacg
cgatattttgaaaaaaggcgctacttttaatcgagaaacaccaggagttc
ccattgcttatacaacaaacttcctaaaagacaatgaattagctgttatt
aaaaacaactcagaatatattgaaacaacttcaaaagcttatacagatgg
aaaaattaacatcgatcactctggaggatacgttgctcaattcaacattt
cttgggatgaagtaaattatgat.

In another embodiment, the LLO protein comprises the sequence SEQ ID NO: 4:

(SEQ ID NO: 4)

M K K I M L V F I T L I L V S L P I A Q Q T E A K
D A S A F N KE N S I S S M A P P A S P P A S P K T
P I E K K H A D E I D K Y I QG L D Y N K N N V L
V Y H G D A V T N V P P R K G Y K D G N E YI V V E
K K K K S I N Q N N A D I Q V V N A I S S L T Y P
G A L VK A N S E L V E N Q P D V L P V K R D S L
T L S I D L P G M T N QD N K I V V K N A T K S N V
N N A V N T L V E R W N E K Y A Q AY P N V S A K
I D Y D D E M A Y S E S Q L I A K F G T A F K A V
NN S L N V N F G A I S E G K M Q E E V I S F K Q I
Y Y N V N V N EP T R P S R F F G K A V T K E Q L
Q A L G V N A E N P P A Y I S SV A Y G R Q V Y L K
L S T N S H S T K V K A A F D A A V S G K SV S G
D V E L T N I I K N S S F K A V I Y G G S A K D E
V Q I I DG N L G D L R D I L K K G A T F N R E T P
G V P I A Y T T N F L KD N E L A V I K N N S E Y
I E T T S K A Y T D G K I N I D H S G GY V A Q F N
I S W D E V N Y D

The first 25 amino acids of the proprotein corresponding to this sequence are the signal sequence and are cleaved from LLO when it is secreted by the bacterium. Thus, in this embodiment, the full length active LLO protein is 504 residues long. In another embodiment, the LLO protein has a sequence set forth in GenBank Accession No. DQ054588, DQ054589, AY878649, U25452, or U25452. In another embodiment, the LLO protein is a variant of an LLO protein. In another embodiment, the LLO protein is a homologue of an LLO protein. Each possibility represents a separate embodiment of the present invention.

In another embodiment, “truncated LLO” or “tLLO” refers to a fragment of LLO that comprises a PEST domain. In another embodiment, the terms refer to an LLO fragment that does not contain the activation domain at the amino terminus and does not include cystine 484. In another embodiment, the LLO fragment consists of a PEST sequence. In another embodiment, the LLO fragment comprises a PEST sequence. In another embodiment, the LLO fragment consists of about the first 400 to 441 amino acids of the 529 amino acid full-length LLO protein. In another embodiment, the LLO fragment is a non-hemolytic form of the LLO protein.

In another embodiment of the methods and compositions of the present invention, a recombinant polypeptide encoded by a nucleic acid sequence of the methods and compositions of the present invention is a fusion protein comprising the chimeric HER2/neu antigen and an additional polypeptide, where in another embodiment, the fusion protein comprises, inter alia, an LLO fragment which in one embodiment is an LM non-hemolytic LLO protein or, in another embodiment, a truncated LLO (Examples herein).

In one embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the present invention.

In another embodiment, a fusion protein of methods and compositions of the present invention comprises a PEST sequence, either from an LLO protein or from another organism, e.g. a prokaryotic organism.

The PEST amino acid (AA) sequence comprises, in another embodiment, a sequence selected from SEQ ID NO: 5-9. In another embodiment, the PEST sequence is a PEST sequence from the Listeria monocytogenes (Lm) ActA protein. In another embodiment, a PEST sequence comprises KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: ⁷), or RGGIPTSEEFSSLNSGDFTDDENSETTEEEIDR (SEQ ID NO: 8). In another embodiment, the PEST sequence is from Streptolysin O protein of Streptococcus sp. In another embodiment, the PEST sequence is from Streptococcus pyogenes Streptolysin O, e.g. KQNTASTETTTTNEQPK (SEQ ID NO: 9) at AA 35-51. In another embodiment, the PEST sequence is from Streptococcus equisimilis Streptolysin O, e.g. KQNTANTETTTTNEQPK (SEQ ID NO: 10) at AA 38-54. In another embodiment, the PEST-like sequence is another PEST AA sequence derived from a prokaryotic organism. In another embodiment, the PEST sequence is any other PEST sequence known in the art. Each possibility represents a separate embodiment of the present invention.

Fusion of an antigen to a PEST sequence of Lm enhanced cell mediated and anti-tumor immunity of the antigen. Thus, fusion of an antigen to other PEST sequences derived from other prokaryotic organisms will also enhance immunogenicity of the antigen. PEST sequence of other prokaryotic organism can be identified in accordance with methods such as described by, for example Rechsteiner and Rogers (1996, Trends Biochem. Sci. 21:267-271) for Lm. Alternatively, PEST AA sequences from other prokaryotic organisms can also be identified based on this method. Other prokaryotic organisms wherein PEST AA sequences would be expected to occur include, but are not limited to, other Listeria species. In another embodiment, the PEST sequence is embedded within the antigenic protein. Thus, in another embodiment, “fusion” refers to an antigenic protein comprising both the antigen and the PEST amino acid sequence either linked at one end of the antigen or embedded within the antigen.

In another embodiment, provided herein is a vaccine comprising a recombinant polypeptide of the present invention. In another embodiment, provided herein is a composition comprising a recombinant polypeptide of the present invention. In another embodiment, provided herein is a vaccine consisting of a recombinant polypeptide of the present invention. In another embodiment, provided herein is a composition consisting of a recombinant polypeptide of the present invention. In another embodiment, the composition is an immunogenic composition.

In another embodiment, provided herein is a nucleotide molecule encoding a recombinant polypeptide of the present invention. In another embodiment, provided herein is a vaccine comprising the nucleotide molecule. In another embodiment, provided herein is a composition comprising the nucleotide molecule.

In another embodiment, provided herein is a nucleotide molecule encoding a recombinant polypeptide of the present invention.

In another embodiment, provided herein is a recombinant polypeptide encoded by the nucleotide molecule of the present invention.

In another embodiment, provided herein is a vaccine comprising a nucleotide molecule or recombinant polypeptide of the present invention.

In another embodiment, provided herein is an immunogenic composition comprising a nucleotide molecule or recombinant polypeptide of the present invention.

In another embodiment, provided herein is a vector comprising a nucleotide molecule or recombinant polypeptide of the present invention.

In another embodiment, provided herein is a recombinant form of Listeria comprising a nucleotide molecule of the present invention.

In another embodiment, provided herein is a vaccine comprising a recombinant form of Listeria of the present invention.

In another embodiment, provided herein is an immunogenic composition comprising a recombinant form of Listeria of the present invention.

In another embodiment, provided herein is a culture of a recombinant form of Listeria of the present invention.

In one embodiment, a vaccine or composition for use in the methods of the present invention comprises a recombinant Listeria monocytogenes, in any form or embodiment as described herein. In one embodiment, the vaccine or composition for use in the present invention consists of a recombinant Listeria monocytogenes of the present invention, in any form or embodiment as described herein. In another embodiment, the vaccine or composition for use in the methods of the present invention consists essentially of a recombinant Listeria monocytogenes of the present invention, in any form or embodiment as described herein.

In one embodiment, the term “comprise” refers to the inclusion of a recombinant Listeria monocytogenes in the vaccine or composition, as well as inclusion of other vaccines, compositions or treatments that may be known in the art. In another embodiment, the term “consisting essentially of” refers to a vaccine, whose functional component is the recombinant Listeria monocytogenes, however, other components of the vaccine or composition may be included that are not involved directly in the therapeutic effect of the vaccine and may, for example, refer to components which facilitate the effect of the recombinant Listeria monocytogenes (e.g. stabilizing, preserving, etc.). In another embodiment, the term “consisting” refers to a vaccine, which contains the recombinant Listeria monocytogenes.

In another embodiment, the methods of the present invention comprise the step of administering a recombinant Listeria monocytogenes, in any form or embodiment as described herein. In one embodiment, the methods of the present invention consist of the step of administering a recombinant Listeria monocytogenes of the present invention, in any form or embodiment as described herein. In another embodiment, the methods of the present invention consist essentially of the step of administering a recombinant Listeria monocytogenes of the present invention, in any form or embodiment as described herein. In one embodiment, the term “comprise” refers to the inclusion of the step of administering a recombinant Listeria monocytogenes in the methods, as well as inclusion of other methods or treatments that may be known in the art. In another embodiment, the term “consisting essentially of” refers to a methods, whose functional component is the administration of recombinant Listeria monocytogenes, however, other steps of the methods may be included that are not involved directly in the therapeutic effect of the methods and may, for example, refer to steps which facilitate the effect of the administration of recombinant Listeria monocytogenes. In one embodiment, the term “consisting” refers to a method of administering recombinant Listeria monocytogenes with no additional steps.

In another embodiment, the Listeria of methods and compositions of the present invention is Listeria monocytogenes. In another embodiment, the Listeria is Listeria ivanovii. In another embodiment, the Listeria is Listeria welshimeri. In another embodiment, the Listeria is Listeria seeligeri. Each type of Listeria represents a separate embodiment of the present invention.

In one embodiment, the Listeria strain of the methods and compositions of the present invention is the ADXS31-164 strain. In another embodiment, ADXS31-164 stimulates the secretion of IFN-γ by the splenocytes from wild type FVB/N mice. Further, the data presented herein show that ADXS31-164 is able to elicit anti-HER2/neu specific immune responses to human epitopes that are located at different domains of the targeted antigen.

In another embodiment, the present invention provides a recombinant form of Listeria comprising a nucleotide molecule encoding a HER2 chimeric protein or a fragment thereof.

In one embodiment, the present invention provides a method of inducing an anti-HER2 immune response in a subject, comprising administering to the subject a recombinant polypeptide comprising an N-terminal fragment of a LLO protein fused to a HER2 chimeric protein or fused to a fragment thereof, thereby inducing an anti-HER2 immune response in a subject.

In one embodiment, the two molecules of the fusion protein (the LLO, ActA fragment or PEST sequence and the antigen) are joined directly. In another embodiment, the two molecules are joined by a short spacer peptide, consisting of one or more amino acids. In one embodiment, the spacer has no specific biological activity other than to join the proteins or to preserve some minimum distance or other spatial relationship between them. In another embodiment, the constituent amino acids of the spacer are selected to influence some property of the molecule such as the folding, net charge, or hydrophobicity. In another embodiment, the two molecules of the protein (the LLO fragment and the antigen) are synthesized separately or unfused. In another embodiment, the two molecules of the protein are synthesized separately from the same nucleic acid. In yet another embodiment, the two molecules are individually synthesized from separate nucleic acids. Each possibility represents a separate embodiment of the present invention.

In one embodiment, nucleic acids encoding the recombinant polypeptides provided herein also encode a signal peptide or sequence. In another embodiment, the fusion protein of methods and compositions of the present invention comprises an LLO signal sequence from LLO. In one embodiment, a heterologous antigen may be expressed through the use of a signal sequence, such as a Listerial signal sequence, for example, the hemolysin signal sequence or the actA signal sequence. Alternatively, for example, foreign genes can be expressed downstream from a L. monocytogenes promoter without creating a fusion protein. In another embodiment, the signal peptide is bacterial (Listerial or non-Listerial). In one embodiment, the signal peptide is native to the bacterium. In another embodiment, the signal peptide is foreign to the bacterium. In another embodiment, the signal peptide is a signal peptide from Listeria monocytogenes, such as a secA1 signal peptide. In another embodiment, the signal peptide is a Usp45 signal peptide from Lactococcus lactis, or a Protective Antigen signal peptide from Bacillus anthracis. In another embodiment, the signal peptide is a secA2 signal peptide, such the p60 signal peptide from Listeria monocytogenes. In addition, the recombinant nucleic acid molecule optionally comprises a third polynucleotide sequence encoding p60, or a fragment thereof. In another embodiment, the signal peptide is a Tat signal peptide, such as a B. subtilis Tat signal peptide (e.g., PhoD). In one embodiment, the signal peptide is in the same translational reading frame encoding the recombinant polypeptide.

In another embodiment, provided herein is a method of inducing an anti-HER2 immune response in a subject, comprising administering to the subject a recombinant nucleotide encoding a recombinant polypeptide comprising an N-terminal fragment of a LLO protein fused to a HER2 chimeric protein or fused to a fragment thereof, thereby inducing an anti-HER2 immune response in a subject.

In one embodiment, provided herein is a method of eliciting an enhanced immune response to a HER2/neu-expressing tumor in a subject, where in another embodiment the method comprises administering to the subject a composition comprising the recombinant Listeria vaccine strain provided herein. In another embodiment, the immune response against the HER2-expressing tumor comprises an immune response to a subdominant epitope of the HER2 protein. In another embodiment, the immune response against the HER2-expressing tumor comprises an immune response to several subdominant epitopes of the HER2protein. In another embodiment, the immune response against the HER2-expressing tumor comprises an immune response to at least 1-5 subdominant epitopes of the HER2protein. In another embodiment, the immune response against the HER2-expressing tumor comprises an immune response to at least 1-10 subdominant epitopes of the HER2protein. In another embodiment, the immune response against the HER2-expressing tumor comprises an immune response to at least 1-17 subdominant epitopes of the HER2protein. In another embodiment, the immune response against the HER2-expressing tumor comprises an immune response to at least 17 subdominant epitopes of the HER2 protein.

Point mutations or amino-acid deletions in the oncogenic protein HER2/neu, have been reported to mediate treatment of resistant tumor cells, when these tumors have been targeted by small fragment Listeria-based vaccines or trastuzumab (a monoclonal antibody against an epitope located at the extracellular domain of the HER2/neu antigen). Described herein is a chimeric HER2/neu based composition which harbors two of the extracellular and one intracellular fragments of HER2/neu antigen showing clusters of MHC-class I epitopes of the oncogene. This chimeric protein, which harbors 3 H2Dq and at least 17 of the mapped human MHC-class I epitopes of the HER2/neu antigen was fused to the first 441 amino acids of the Listeria-monocytogenes listeriolysin O protein and expressed and secreted by the Listeria monocytogenes attenuated strain LmddA.

Previous reports have shown that when HER2/neu transgenic mice were immunized with Listeria-based vaccines expressing and secreting small fragments of the HER2/neu antigen separately (each of which harbored only one H2Dq epitope of the HER2/neu oncogene), HER2/neu over-expressing tumors could escape due to mutations in those epitopes of the HER2/neu antigen targeted by each vaccine (see Singh R, Paterson Y. Immunoediting sculpts tumor epitopes during immunotherapy. Cancer Res 2007; 67: 1887-92). Demonstrated herein is the unexpected result that when three or more epitopes of the HER2/neu protein are incorporated in a chimeric vaccine, it can eliminate the selection and escape of these tumors by escape mutations Immunization with the novel HER2/neu chimeric Listeria vaccines did not result in any escape mutations that could be associated with point mutations or amino acid deletions in the HER2/neu antigen (see Example 4 herein).

In one embodiment, provided herein is a method of engineering a Listeria vaccine strain to express a HER2 chimeric protein or recombinant polypeptide expressing the chimeric protein, the method comprising transforming a Listeria strain with a nucleic acid molecule. In another embodiment, the nucleic acid molecule comprises a first open reading frame encoding a polypeptide, wherein the polypeptide comprises a HER2/neu chimeric antigen. In another embodiment, the nucleic acid molecule further comprises a second open reading frame encoding a metabolic enzyme, and wherein said metabolic enzyme complements an endogenous gene that is lacking in the chromosome of the recombinant Listeria strain, thereby engineering a Listeria vaccine strain to express a HER2 chimeric protein.

In one embodiment, the methods and compositions provided herein further comprise an adjuvant, where in another embodiment, the adjuvant comprises a granulocyte/macrophage colony-stimulating factor (GM-CSF) protein, a nucleotide molecule encoding a GM-CSF protein, saponin QS21, monophosphoryl lipid A, an unmethylated CpG-containing oligonucleotide or any adjuvant known in the art.

In one embodiment, attenuated Listeria strains, such as LM delta-actA mutant (Brundage et al, 1993, Proc. Natl. Acad. Sci., USA, 90:11890-11894), L. monocytogenes delta-plcA (Camilli et al, 1991, J. Exp. Med., 173:751-754), or delta-ActA, delta INL-b (Brockstedt et 5 al, 2004, PNAS, 101:13832-13837) are used in the present invention. In another embodiment, attenuated Listeria strains are constructed by introducing one or more attenuating mutations, as will be understood by one of average skill in the art when equipped with the disclosure herein. Examples of such strains include, but are not limited to Listeria strains auxotrophic for aromatic amino acids (Alexander et al, 1993, Infection and Immunity 10 61:2245-2248) and mutant for the formation of lipoteichoic acids (Abachin et al, 2002, Mol. Microbiol. 43:1-14) and those attenuated by a lack of a virulence gene (see examples herein).

In another embodiment, a nucleic acid molecule of the methods and compositions of the present invention is operably linked to a promoter/regulatory sequence. In another embodiment, the first open reading frame of methods and compositions of the present invention is operably linked to a promoter/regulatory sequence. In another embodiment, the nucleic acid molecule comprises a second open reading frame operably linked to a promoter/regulatory sequence. In another embodiment, each of the open reading frames are operably linked to a promoter/regulatory sequence. Each possibility represents a separate embodiment of the present invention.

The skilled artisan, when equipped with the present disclosure and the methods provided herein, will readily understand that different transcriptional promoters, terminators, carrier vectors or specific gene sequences (e.g. those in commercially available cloning vectors) can be used successfully in methods and compositions of the present invention. As is contemplated in the present invention, these functionalities are provided in, for example, the commercially available vectors known as the pUC series. In another embodiment, non-essential DNA sequences (e.g. antibiotic resistance genes) are removed. Each possibility represents a separate embodiment of the present invention. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art.

Another embodiment is a plasmid such as pCR2.1 (Invitrogen, La Jolla, CA), which is a prokaryotic expression vector with a prokaryotic origin of replication and promoter/regulatory elements to facilitate expression in a prokaryotic organism. In another embodiment, extraneous nucleotide sequences are removed to decrease the size of the plasmid and increase the size of the cassette that can be placed therein.

Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

Antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, DC; Miller, 1992, A Short Course in Bacterial Genetics, Cold Spring Harbor Laboratory Press, Cold Spring Harbor, N.Y.) In another embodiment, the Listeria vaccine strain of the present invention is transformed by electroporation. Each method represents a separate embodiment of the present invention.

In another embodiment, conjugation is used to introduce genetic material and/or plasmids into bacteria. Methods for conjugation are well known in the art, and are described, for example, in Nikodinovic J et al. (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102 (35):12554-9). Each method represents a separate embodiment of the present invention.

“Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

Plasmids and other expression vectors useful in the present invention are described elsewhere herein, and can include such features as a promoter/regulatory sequence, an origin of replication for gram negative and gram positive bacteria, an isolated nucleic acid encoding a fusion protein and an isolated nucleic acid encoding an amino acid metabolism gene. Further, an isolated nucleic acid encoding a fusion protein and an amino acid metabolism gene will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann. Rev. Genet. 18:415-442). Further examples of promoter/regulatory elements contemplated in the present invention include, but are not limited to the Listerial prfA promoter, the Listerial hly promoter, the Listerial p60 promoter and the Listerial actA promoter (GenBank Acc. No. NC_003210) or fragments thereof.

In another embodiment, a plasmid of methods and compositions of the present invention comprises a gene encoding a fusion protein. In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In another embodiment, the present invention further comprises a phage based chromosomal integration system for clinical applications. A host strain that is auxotrophic for essential enzymes, including, but not limited to, d-alanine racemase will be used, for example Lmdal(−)dat(−). In another embodiment, in order to avoid a “phage curing step,” a phage integration system based on PSA is used (Lauer, et al., 2002 J Bacteriol, 184:4177-4186). This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain will be complemented.

The recombinant proteins of the present invention are synthesized, in another embodiment, using recombinant DNA methodology. This involves, in one embodiment, creating a DNA sequence that encodes the fusion protein, placing the DNA in an expression cassette, such as the plasmid of the present invention, under the control of a particular promoter/regulatory element, and expressing the protein. DNA encoding the fusion protein (e.g. non-hemolytic LLO/antigen) of the present invention is prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 15 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then ligated to produce the desired DNA sequence.

In another embodiment, DNA encoding the fusion protein or the recombinant protein of the present invention is cloned using DNA amplification methods such as polymerase chain reaction (PCR). Thus, the gene for non-hemolytic LLO is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the non-hemolytic LLO and antigen sequences and insertion into a plasmid or vector produces a vector encoding non-hemolytic LLO joined to a terminus of the antigen. The two molecules are joined either directly or by a short spacer introduced by the restriction site.

In another embodiment, the molecules are separated by a peptide spacer consisting of one or more amino acids, generally the spacer will have no specific biological activity other than to join the proteins or to preserve some minimum distance or other spatial relationship between them. In another embodiment, the constituent AA of the spacer are selected to influence some property of the molecule such as the folding, net charge, or hydrophobicity. In another embodiment, the nucleic acid sequences encoding the fusion or recombinant proteins are transformed into a variety of host cells, including E. coli, other bacterial hosts, such as Listeria, yeast, and various higher eukaryotic cells such as the COS, CHO and HeLa cells lines and myeloma cell lines. The recombinant fusion protein gene will be operably linked to appropriate expression control sequences for each host. Promoter/WO regulatory sequences are described in detail elsewhere herein. In another embodiment, the plasmid further comprises additional promoter regulatory elements, as well as a ribosome binding site and a transcription termination signal. For eukaryotic cells, the control sequences will include a promoter and an enhancer derived from e g immunoglobulin genes, SV40, cytomegalovirus, etc., and a polyadenylation sequence. In another embodiment, the sequences include splice donor and acceptor sequences.

In one embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In another embodiment, in order to select for an auxotrophic bacterium comprising the plasmid, transformed auxotrophic bacteria are grown on a media that will select for expression of the amino acid metabolism gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with this invention.

In one embodiment, provided herein is a method of impeding a growth of a HER2-expressing tumor in a subject, wherein and in another embodiment, the method comprises the step of administering to the subject a composition comprising the recombinant Listeria vaccine strain described herein.

In another embodiment, provided herein is a method of impeding or delaying metastatic disease origination from a HER2-expressing tumor in a subject, wherein and in another embodiment, the method comprises the step of administering to the subject a composition comprising the recombinant Listeria vaccine strain described herein.

In another embodiment, provided herein is a method of eliciting an enhanced immune response to a HER2/neu-expressing tumor in a subject, wherein and in another embodiment, the method comprises the step of administering to the subject a composition comprising the recombinant Listeria vaccine strain described herein. In yet another embodiment, the immune response against the HER2/neu-expressing tumor comprises an immune response to at least one subdominant epitope of the HER2/neu protein.

In one embodiment, provided herein is a method of preventing an escape mutation in the treatment of HER2/neu expressing tumors, wherein and in another embodiment, the method comprises the step of administering to said subject a composition comprising the recombinant Listeria vaccine strain provided herein.

In another embodiment, provided herein is a method of preventing the onset of a HER2/neu antigen-expressing tumor in a subject, wherein and in another embodiment, the method comprises the step of administering to the subject a composition comprising the recombinant Listeria vaccine strain provided herein.

In one embodiment, provided herein is a method of decreasing the frequency of intra-tumoral T regulatory cells, wherein and in another embodiment, the method comprises the step of administering to the subject a composition comprising the recombinant Listeria vaccine strain provided herein.

In another embodiment, provided herein is a method of decreasing the frequency of intra-tumoral T regulatory cells, wherein and in another embodiment, the method comprises the step of administering to the subject a composition comprising the recombinant Listeria vaccine strain provided herein.

In one embodiment, provided herein is a method of decreasing the frequency of intra-tumoral myeloid derived suppressor cells, wherein and in another embodiment, the method comprises the step of administering to the subject a composition comprising the recombinant Listeria vaccine strain provided herein.

In another embodiment, provided herein is a method of decreasing the frequency of myeloid derived suppressor cells, wherein and in another embodiment, the method comprises the step of administering to the subject a composition comprising the recombinant Listeria vaccine strain provided herein.

In one embodiment, provided herein a method of preventing the formation of a HER2/neu-expressing tumor in a subject, wherein and in another embodiment, the method comprises the step of administering to the subject a composition comprising the recombinant Listeria vaccine strain provided herein. In another embodiment, provided herein is a method of preventing the formation of a metastatic disease originating from an Her2/neu-expressing tumor in a subject, wherein and in another embodiment, the method comprises the step of administering to the subject a composition comprising the recombinant Listeria vaccine strain the provided herein. In one embodiment, provided herein is a method of treating a Her2/neu-expressing tumor in a subject, wherein and in another embodiment, the method comprises the step of administering to the subject a composition comprising the recombinant Listeria vaccine strain provided herein. In another embodiment, provided herein is a method of treating a metastatic disease coming from a Her2/neu-expressing tumor in a subject, wherein and in another embodiment, the method comprises the step of administering to the subject a composition comprising the recombinant Listeria vaccine strain provided herein. In one embodiment, provided herein is a method of administering a composition of the present invention. In another embodiment, provided herein is a method of administering a vaccine of the present invention. In another embodiment, provided herein is a method of administering the recombinant polypeptide or recombinant nucleotide of the present invention. In another embodiment, the step of administering the composition, vaccine, recombinant polypeptide or recombinant nucleotide of the present invention is performed with an attenuated recombinant form of Listeria comprising the composition, vaccine, recombinant nucleotide or expressing the recombinant polypeptide, each in its own discrete embodiment. In another embodiment, the administering is performed with a different attenuated bacterial vector. In another embodiment, the administering is performed with a DNA vaccine (e.g. a naked DNA vaccine). In another embodiment, administration of a recombinant polypeptide of the present invention is performed by producing the protein recombinantly, then administering the recombinant protein to a subject. Each possibility represents a separate embodiment of the present invention.

In one embodiment, repeat administrations (booster doses) of compositions of this invention may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In another embodiment, the immune response elicited by methods and compositions of the present invention comprises a CD8⁺ T cell-mediated response. In another embodiment, the immune response consists primarily of a CD8⁺ T cell-mediated response. In another embodiment, the only detectable component of the immune response is a CD8⁺ T cell-mediated response.

In another embodiment, the immune response elicited by methods and compositions provided herein comprises a CD4⁺ T cell-mediated response. In another embodiment, the immune response consists primarily of a CD4⁺ T cell-mediated response. In another embodiment, the only detectable component of the immune response is a CD4⁺ T cell-mediated response. In another embodiment, the CD4⁺ T cell-mediated response is accompanied by a measurable antibody response against the antigen. In another embodiment, the CD4⁺ T cell-mediated response is not accompanied by a measurable antibody response against the antigen.

In another embodiment, the present invention provides a method of inducing a CD8⁺ T cell-mediated immune response in a subject against a subdominant CD8⁺ T cell epitope of an antigen, comprising the steps of (a) fusing a nucleotide molecule encoding the Her2-neu chimeric antigen or a fragment thereof to a nucleotide molecule encoding an N-terminal fragment of a LLO protein, thereby creating a recombinant nucleotide encoding an LLO-antigen fusion protein; and (b) administering the recombinant nucleotide or the LLO-antigen fusion to the subject; thereby inducing a CD8⁺ T cell-mediated immune response against a subdominant CD8⁺ T cell epitope of an antigen.

In one embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, the immune response elicited by the methods and compositions provided herein comprises an immune response to at least one subdominant epitope of the antigen. In another embodiment, the immune response does not comprise an immune response to a subdominant epitope. In another embodiment, the immune response consists primarily of an immune response to at least one subdominant epitope. In another embodiment, the only measurable component of the immune response is an immune response to at least one subdominant epitope. Each type of immune response represents a separate embodiment of the present invention.

In one embodiment, methods of this invention break tolerance in a subject to a HER2/expressing tumor or cancer in said subject, wherein and in another embodiment, the method comprises the step of administering to the subject a composition comprising the recombinant Listeria vaccine strain provided herein.

Methods of measuring immune responses are well known in the art, and include, e.g. measuring suppression of tumor growth, flow cytometry, target cell lysis assays (e.g. chromium release assay), the use of tetramers, and others. Each method represents a separate embodiment of the present invention.

In another embodiment, the present invention provides a method of delaying or inhibiting a metastatic disease emanating from a Her-2-expressing tumor in a subject, wherein and in another embodiment, the method comprises administering to the subject a recombinant polypeptide comprising an N-terminal fragment of a LLO protein fused to the HER2 chimeric protein or a fragment thereof or a recombinant nucleotide encoding the recombinant polypeptide, wherein the subject mounts an immune response against the HER2-expressing tumor, thereby delaying or inhibiting the metastatic disease emanating from a HER2-expres sing tumor in a subject.

In another embodiment, the present invention provides a method of improving an antigenicity of a HER2 chimeric protein, wherein and in another embodiment, the method comprises the step of fusing a nucleotide encoding an N-terminal fragment of a LLO protein to a nucleotide encoding the Her-2 protein or a fragment thereof to create a recombinant polypeptide, thereby improving an antigenicity of a HER2 chimeric protein.

In another embodiment, provided herein is a method of improving an antigenicity of a HER2 chimeric protein, wherein and in another embodiment, the method comprises engineering a Listeria strain to express the recombinant nucleotide. In another embodiment, a different bacterial vector is used to express the recombinant nucleotide. In another embodiment, the bacterial vector is attenuated. In another embodiment, a DNA vaccine (e.g. a naked DNA vaccine) is used to express the recombinant nucleotide. In another embodiment, administration of the LLO-HER2 chimera fusion peptide encoded by the nucleotide is performed by producing the protein recombinantly, then administering the recombinant protein to a subject. Each possibility represents a separate embodiment of the present invention.

In one embodiment, the present invention provides a method for “epitope spreading” of a tumor. In another embodiment, the immunization using the compositions and methods provided herein induce epitope spreading onto other tumors bearing antigens other than the antigen carried in the vaccine of the present invention.

In another embodiment, the dominant epitope or subdominant epitope is dominant or subdominant, respectively, in the subject being treated. In another embodiment, the dominant epitope or subdominant epitope is dominant or subdominant in a population being treated.

In one embodiment, provided herein is a method of treating, suppressing, or inhibiting a cancer or a tumor growth in a subject by epitope spreading wherein and in another embodiment, said cancer is associated with expression of an antigen or fragment thereof comprised in the composition of the present invention. In another embodiment, the method comprises administering to said subject a composition comprising the recombinant polypeptide, recombinant Listeria, or recombinant vector of the present invention. In yet another embodiment, the subject mounts an immune response against the antigen-expressing cancer or the antigen-expressing tumor, thereby treating, suppressing, or inhibiting a cancer or a tumor growth in a subject.

“Dominant CD8⁺ T cell epitope,” in one embodiment, refers to an epitope that is recognized by over 30% of the antigen-specific CD8⁺ T cells that are elicited by vaccination, infection, or a malignant growth with a protein or a pathogen or cancer cell containing the protein. In another embodiment, the term refers to an epitope recognized by over 35% of the antigen-specific CD8⁺ T cells that are elicited thereby. In another embodiment, the term refers to an epitope recognized by over 40% of the antigen-specific CD8⁺ T cells. In another embodiment, the term refers to an epitope recognized by over 45% of the antigen-specific CD8⁺ T cells. In another embodiment, the term refers to an epitope recognized by over 50% of the antigen-specific CD8⁺ T cells. In another embodiment, the term refers to an epitope recognized by over 55% of the antigen-specific CD8⁺ T cells. In another embodiment, the term refers to an epitope recognized by over 60% of the antigen-specific CD8⁺ T cells. In another embodiment, the term refers to an epitope recognized by over 65% of the antigen-specific CD8⁺ T cells. In another embodiment, the term refers to an epitope recognized by over 70% of the antigen-specific CD8⁺ T cells. In another embodiment, the term refers to an epitope recognized by over 75% of the antigen-specific CD8⁺ T cells. In another embodiment, the term refers to an epitope recognized by over 80% of the antigen-specific CD8⁺ T cells. In another embodiment, the term refers to an epitope recognized by over 85% of the antigen-specific CD8⁺ T cells. In another embodiment, the term refers to an epitope recognized by over 90% of the antigen-specific CD8⁺ T cells. In another embodiment, the term refers to an epitope recognized by over 95% of the antigen-specific CD8⁺ T cells. In another embodiment, the term refers to an epitope recognized by over 96% of the antigen-specific CD8⁺ T cells. In another embodiment, the term refers to an epitope recognized by over 97% of the antigen-specific CD8⁺ T cells. In another embodiment, the term refers to an epitope recognized by over 98% of the antigen-specific CD8⁺ T cells.

“Subdominant CD8⁺ T cell epitope,” in one embodiment, refers to an epitope recognized by fewer than 30% of the antigen-specific CD8⁺ T cells that are elicited by vaccination, infection, or a malignant growth with a protein or a pathogen or cancer cell containing the protein. In another embodiment, the term refers to an epitope recognized by fewer than 28% of the antigen-specific CD8⁺ T cells. In another embodiment, the term refers to an epitope recognized by over 26% of the antigen-specific CD8⁺ T cells. In another embodiment, the term refers to an epitope recognized by fewer than 24% of the antigen-specific CD8⁺ T cells. In another embodiment, the term refers to an epitope recognized by over 22% of the antigen-specific CD8⁺ T cells. In another embodiment, the term refers to an epitope recognized by fewer than 20% of the antigen-specific CD8⁺ T cells. In another embodiment, the term refers to an epitope recognized by over 18% of the antigen-specific CD8⁺ T cells. In another embodiment, the term refers to an epitope recognized by fewer than 16% of the antigen-specific CD8⁺ T cells. In another embodiment, the term refers to an epitope recognized by over 14% of the antigen-specific CD8⁺ T cells. In another embodiment, the term refers to an epitope recognized by over 12% of the antigen-specific CD8⁺ T cells. In another embodiment, the term refers to an epitope recognized by fewer than 10% of the antigen-specific CD8⁺ T cells. In another embodiment, the term refers to an epitope recognized by over 8% of the antigen-specific CD8⁺ T cells. In another embodiment, the term refers to an epitope recognized by fewer than 6% of the antigen-specific CD8⁺ T cells. In another embodiment, the term refers to an epitope recognized by fewer than 5% of the antigen-specific CD8⁺ T cells. In another embodiment, the term refers to an epitope recognized by over 4% of the antigen-specific CD8⁺ T cells. In another embodiment, the term refers to an epitope recognized by fewer than 3% of the antigen-specific CD8⁺ T cells. In another embodiment, the term refers to an epitope recognized by fewer than 2% of the antigen-specific CD8⁺ T cells. In another embodiment, the term refers to an epitope recognized by less than 1% of the antigen-specific CD8⁺ T cells. In another embodiment, the term refers to an epitope recognized by less than 0.5% of the antigen-specific CD8⁺ T cells.

Each type of the dominant epitope and subdominant epitope represents a separate embodiment of the present invention.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In one embodiment, “non-tumor cell” refers to a cell outside the body of the tumor. In another embodiment, “non-tumor cell” refers to a non-malignant cell. In another embodiment, “non-tumor cell” refers to a non-transformed cell. In another embodiment, the non-tumor cell is a somatic cell. In another embodiment, the non-tumor cell is a germ cell. Each possibility represents a separate embodiment of the present invention.

“Detectable level” refers, in one embodiment, to a level that is detectable when using a standard assay. In one embodiment, the assay is an immunological assay. In one embodiment, the assay is enzyme-linked immunoassay (ELISA). In another embodiment, the assay is Western blot. In another embodiment, the assay is FACS. It is to be understood by a skilled artisan that any other assay available in the art can be used in the methods provided herein. In another embodiment, a detectable level is determined relative to the background level of a particular assay. Methods for performing each of these techniques are well known to those skilled in the art, and each technique represents a separate embodiment of the present invention.

In one embodiment, vaccination with recombinant antigen-expressing LM induces epitope spreading. In another embodiment, vaccination with LLO-antigen fusions, even outside the context of Her2, induces epitope spreading as well. Each possibility represents a separate embodiment of the present invention.

In another embodiment, the present invention provides a method of impeding a growth of an HER2-expressing tumor in a subject, comprising administering to the subject a recombinant polypeptide comprising an N-terminal fragment of a LLO protein fused to a HER2 chimeric antigen, wherein the antigen has one or more subdominant CD8⁺ T cell epitopes, wherein the subject mounts an immune response against the antigen-expressing tumor, thereby impeding a growth of an HER2-expressing tumor in a subject. In another embodiment, the antigen does not contain any of the dominant CD8⁺ T cell epitopes. In another embodiment, provided herein is a method of impeding a growth on a HER2-expressing tumor in a subject, comprising administering to the subject a recombinant form of Listeria comprising a recombinant nucleotide encoding the recombinant polypeptide provided herein.

In another embodiment, the present invention provides a method for inducing formation of cytotoxic T cells in a host having cancer, comprising administering to the host a composition of the present invention, thereby inducing formation of cytotoxic T cells in a host having cancer.

In another embodiment, the present invention provides a method of reducing an incidence of cancer, comprising administering a composition of the present invention. In another embodiment, the present invention provides a method of ameliorating cancer, comprising administering a composition of the present invention. Each possibility represents a separate embodiment of the present invention.

In one embodiment, the composition is administered to the cells of the subject ex vivo; in another embodiment, the composition is administered to the cells of a donor ex vivo; in another embodiment, the composition is administered to the cells of a donor in vivo, and then is transferred to the subject. Each possibility represents a separate embodiment of the present invention.

In one embodiment, the cancer treated by a method of the present invention is breast cancer. In another embodiment, the cancer is a Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is a CNS carcinoma. In another embodiment, the cancer is an endometrial carcinoma. In another embodiment, the cancer is a bladder cancer. In another embodiment, the cancer is mesothelioma. In another embodiment, the cancer is malignant mesothelioma (MM). In another embodiment, the cancer is a head and neck cancer. In another embodiment, the cancer is a prostate carcinoma. In another embodiment, the cancer is osteosarcoma. In another embodiment, the cancer is a HER2/neu expressing osteosarcoma. In another embodiment, the osteosarcoma is canine osteosarcoma. In another embodiment, the osteosarcoma is localized osteosarcoma. In another embodiment, the osteosarcoma is metastatic osteosarcoma. In another embodiment, the osteosarcoma is high grade osteosarcoma. In another embodiment, the osteosarcoma is canine appendicular osteosarcoma.

In another embodiment of the methods of the present invention, the subject mounts an immune response against the antigen-expressing tumor or target antigen, thereby mediating the anti-tumor effects.

In another embodiment, the present invention provides an immunogenic composition for treating cancer, the composition comprising a fusion of a truncated LLO to a HER2 chimeric protein. In another embodiment, the immunogenic composition further comprises a Listeria strain expressing the fusion.

In another embodiment, the present invention provides an immunogenic composition for treating cancer, the composition comprising a Listeria strain expressing a HER2 chimeric protein.

In one embodiment, a treatment protocol of the present invention is therapeutic. In another embodiment, the protocol is prophylactic. In another embodiment, the vaccines or compositions of the present invention are used to protect people at risk for cancer such as breast cancer or other types of HER2-containing tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the present invention are administered so that the CTL response to a tumor antigen of the vaccine destroys remaining metastases and prolongs remission from the cancer. In another embodiment, vaccines are used as a cancer immunotherapy in combination with surgery, conventional chemotherapy or radiation treatment. In another embodiment, such combination treatment is used in subjects that cannot undergo amputation. In another embodiment, such combination treatment is used in subjects with primary osteosarcoma that cannot undergo amputation. In another embodiment, vaccines of the present invention are used to effect the growth of previously established tumors and to kill existing tumor cells.

In one embodiment, a “tumor antigen or fragment thereof,” “tumor-associated antigen or fragment thereof,” “heterologous antigen or fragment thereof,” or “antigen peptide or fragment thereof” are used interchangeably herein and include any antigen known in the art including tumor antigens, angiogenic antigens, or infectious disease antigens. In another embodiment, the antigen is a self-antigen.

In one embodiment, the antigen provided herein is derived is a tumor-associated antigen, which in one embodiment, is one of the following tumor antigens: a survivin, a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods as provided herein are melanoma-associated antigens, which in one embodiment are TRP-2, MAGE-1, MAGE-3, gp-100, tyrosinase, HSP-70, beta-HCG, or a combination thereof. Other tumor-associated antigens known in the art are also contemplated in the present invention.

In another embodiment, the antigen or fragment thereof is derived from an antigen selected from a HPV-E7 (from either an HPV16 or HPV18 strain), a HPV-E6 (from either an HPV16 or HPV18 strain), Her-2/neu, NY-ESO-1, telomerase (TERT, SCCE, CEA, LMP-1, p53, carboxic anhydrase IX (CAIX), PSMA, a prostate stem cell antigen (PSCA), a HMW-MAA, WT-1, HIV-1 Gag, Proteinase 3, Tyrosinase related protein 2, PSA (prostate-specific antigen), EGFR-III, survivin, baculoviral inhibitor of apoptosis repeat-containing 5 (BIRC5), LMP-1, p53, PSMA, PSCA, Muc1, PSA (prostate-specific antigen), or a combination thereof.

In another embodiment, the compositions and methods of this invention are used for vaccinating against a tumor or a cancer.

In one embodiment, a treatment protocol of the present invention is therapeutic. In another embodiment, the protocol is prophylactic. In another embodiment, the vaccines or compositions of the present invention are used to protect people at risk for cancer such as breast cancer or other types of HER2-containing tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the present invention are administered so that the CTL response to the tumor antigen of the vaccine destroys remaining metastases and prolongs remission from the cancer. In another embodiment, vaccines are used as a cancer immunotherapy in combination with surgery, or conventional chemotherapy. In another embodiment, such combination treatment is used in subjects that cannot undergo amputation. In another embodiment, such combination treatment is used in subjects with primary osteosarcoma that cannot undergo amputation. In another embodiment, vaccines of the present invention are used to effect the growth of previously established tumors and to kill existing tumor cells.

In another embodiment, the vaccines and immunogenic compositions utilized in any of the methods described above have any of the characteristics of vaccines and immunogenic compositions of the present invention. Each characteristic represents a separate embodiment of the present invention.

Various embodiments of dosage ranges are contemplated by this invention. In one embodiment, in the case of vaccine vectors, the dosage is in the range of 0.4 LD₅₀/dose. In another embodiment, the dosage is from about 0.4-4.9 LD₅₀/dose. In another embodiment the dosage is from about 0.5-0.59 LD₅₀/dose. In another embodiment the dosage is from about 0.6-0.69 LD₅₀/dose. In another embodiment the dosage is from about 0.7-0.79 LD₅₀/dose. In another embodiment the dosage is about 0.8 LD₅₀/dose. In another embodiment, the dosage is 0.4 LD₅₀/dose to 0.8 of the LD₅₀/dose.

In another embodiment, the dosage is 10⁷ bacteria/dose. In another embodiment, the dosage is 1.5×10⁷ bacteria/dose. In another embodiment, the dosage is 2×10⁷ bacteria/dose. In another embodiment, the dosage is 3×10⁷ bacteria/dose. In another embodiment, the dosage is 4×10⁷ bacteria/dose. In another embodiment, the dosage is 6×10⁷ bacteria/dose. In another embodiment, the dosage is 8×10⁷ bacteria/dose. In another embodiment, the dosage is 1×10⁸ bacteria/dose. In another embodiment, the dosage is 1.5×10⁸ bacteria/dose. In another embodiment, the dosage is 2×10⁸ bacteria/dose. In another embodiment, the dosage is 3×10⁸ bacteria/dose. In another embodiment, the dosage is 4×10⁸ bacteria/dose. In another embodiment, the dosage is 6×10⁸ bacteria/dose. In another embodiment, the dosage is 8×10⁸ bacteria/dose. In another embodiment, the dosage is 1×10⁹ bacteria/dose. In another embodiment, the dosage is 1.5×10⁹ bacteria/dose. In another embodiment, the dosage is 2×10⁹ bacteria/dose. In another embodiment, the dosage is 3×10⁹ bacteria/dose. In another embodiment, the dosage is 5×10⁹ bacteria/dose. In another embodiment, the dosage is 6×10⁹ bacteria/dose. In another embodiment, the dosage is 8×10⁹ bacteria/dose. In another embodiment, the dosage is 1×10¹⁰ bacteria/dose. In another embodiment, the dosage is 1.5×10¹⁰ bacteria/dose. In another embodiment, the dosage is 2×10¹⁰ bacteria/dose. In another embodiment, the dosage is 3×10¹⁰ bacteria/dose. In another embodiment, the dosage is 5×10¹⁰ bacteria/dose. In another embodiment, the dosage is 6×10¹⁰ bacteria/dose. In another embodiment, the dosage is 8×10¹⁰ bacteria/dose. In another embodiment, the dosage is 8×10⁹ bacteria/dose. In another embodiment, the dosage is 1×10¹¹ bacteria/dose. In another embodiment, the dosage is 1.5×10¹¹ bacteria/dose. In another embodiment, the dosage is 2×10¹¹ bacteria/dose. In another embodiment, the dosage is 3×10¹¹ bacteria/dose. In another embodiment, the dosage is 5×10¹¹ bacteria/dose. In another embodiment, the dosage is 6×10¹¹ bacteria/dose. In another embodiment, the dosage is 8×10¹¹ bacteria/dose. In another embodiment, the dosage is 5.0×10⁸ bacteria/dose. In another embodiment, the dosage is 3.3×10⁹ bacteria/dose. In another embodiment, a composition for the use in the methods provided herein comprises 3.3×10⁹ Listeria/dose. Each possibility represents a separate embodiment of the present invention.

In one embodiment, a vaccine or immunogenic composition of the present invention is administered alone to a subject. In another embodiment, the vaccine or immunogenic composition is administered together with another cancer therapy. Each possibility represents a separate embodiment of the present invention.

The recombinant Listeria of methods and compositions of the present invention is, in one embodiment, stably transformed with a construct encoding a HER2 chimeric antigen or an LLO-HER2 chimeric antigen fusion. In one embodiment, the construct contains a polylinker to facilitate further subcloning. Several techniques for producing recombinant Listeria are known.

In one embodiment, the construct or nucleic acid molecule is integrated into the Listerial chromosome using homologous recombination. Techniques for homologous recombination are well known in the art, and are described, for example, in Baloglu S, Boyle SM, et al (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant LM strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow MY et al, Construction, characterization, and use of two Listeria monocytogenes site-specific phage integration vectors. J Bacteriol 2002;184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, one of various promoters is used to express the antigen or fusion protein containing same. In one embodiment, an Lm promoter is used, e.g. promoters for the genes hly, actA, pica, plcB and mpl, which encode the Listerial proteins hemolysin, actA, phosphotidylinositol-specific phospholipase, phospholipase C, and metalloprotease, respectively. Each possibility represents a separate embodiment of the present invention.

In another embodiment, methods and compositions of the present invention utilize a homologue of a HER2 chimeric protein or LLO sequence of the present invention. In another embodiment, the methods and compositions of the present invention utilize a HER2 chimeric protein from a non-human mammal. The terms “homology,” “homologous,” etc., when in reference to any protein or peptide, refer in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

In another embodiment, the present invention provides an isolated nucleic acid encoding a signal peptide or a recombinant polypeptide or fusion protein of the present invention. In one embodiment, the isolated nucleic acid comprises a sequence sharing at least 65% homology with a nucleic acid encoding the signal peptide or the recombinant polypeptide or the fusion protein of the present invention. In another embodiment, the isolated nucleic acid comprises a sequence sharing at least 75% homology with a nucleic acid encoding the signal peptide or the recombinant polypeptide or the fusion protein of the present invention. In another embodiment, the isolated nucleic acid comprises a sequence sharing at least 85% homology with a nucleic acid encoding the signal peptide or the recombinant polypeptide or the fusion protein of the present invention. In another embodiment, the isolated nucleic acid comprises a sequence sharing at least 90% homology with a nucleic acid encoding the signal peptide or the recombinant polypeptide or the fusion protein of the present invention. In another embodiment, the isolated nucleic acid comprises a sequence sharing at least 95% homology with a nucleic acid encoding the signal peptide or the recombinant polypeptide or the fusion protein of the present invention. In another embodiment, the isolated nucleic acid comprises a sequence sharing at least 97% homology with a nucleic acid encoding the signal peptide or the recombinant polypeptide or the fusion protein of the present invention. In another embodiment, the isolated nucleic acid comprises a sequence sharing at least 99% homology with a nucleic acid encoding the signal peptide or the recombinant polypeptide or the fusion protein of the present invention.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from a sequence (nucleic acid or amino acid sequence) provided herein of greater than 65%. In another embodiment, “homology” refers to identity to a sequence selected from a sequence provided herein of greater than 70%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaC1, 15 mM trisodium citrate), 50 mM sodium phosphate (pH 7. 6), 5× Denhardt's solution, 10% dextran sulfate, and 20 μg/ml denatured, sheared salmon sperm DNA.

In one embodiment of the present invention, “nucleic acids” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The terms “contacting” or “administering,” in one embodiment, refer to directly contacting the cancer cell or tumor with a composition of the present invention. In another embodiment, the terms refer to indirectly contacting the cancer cell or tumor with a composition of the present invention. In another embodiment, methods of the present invention include methods in which the subject is contacted with a composition of the present invention after which the composition is brought in contact with the cancer cell or tumor by diffusion or any other active transport or passive transport process known in the art by which compounds circulate within the body. In another embodiment, methods of this invention may include at least a single administration of a composition of this invention, wherein in another embodiment, methods of this invention may include multiple administrations of a composition of this invention. Each possibility represents a separate embodiment of the present invention.

In another embodiment, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals or organisms. This can be readily carried out by using hybridization probes to identify the same genetic locus in a variety of individuals or organisms. Any and all such nucleotide variations and resulting amino acid polymorphisms or variations that are the result of natural allelic variation and that do not alter the functional activity are intended to be within the scope of the invention.

### Pharmaceutical Compositions

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of the methods and compositions provided herein, the vaccines or compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelatin capsule.

In another embodiment, the vaccines or compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing,” “suppressing” and “inhibiting” refer in another embodiment to lessening or decreasing. Each possibility represents a separate embodiment of the present invention.

The term “about” as used herein means in quantitative terms plus or minus 5%, or in another embodiment plus or minus 10%, or in another embodiment plus or minus 15%, or in another embodiment plus or minus 20%.

It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

In one embodiment, the term “subject” also encompasses pet dogs and cats, including dogs and cats that cannot undergo amputation. In another embodiment, the term “subject” also encompasses humans that cannot undergo surgery. In another embodiment, the term “subject” also encompasses humans that cannot undergo amputation. In another embodiment, the term “subject” also encompasses a human child.

It will be appreciated by the skilled artisan that the term “mammal” for purposes of treatment refers to any animal classified as a mammal, including, but not limited to, humans, domestic and farm animals, and zoo, sports, or pet animals, such as canines, including dogs, and horses, cats, cattle, pigs, sheep, etc.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a vaccine provided herein for purposes of treatment of tumor may be determined empirically and in a routine manner.

The following examples are presented in order to more fully illustrate the preferred embodiments of the invention. They should in no way be construed, however, as limiting the broad scope of the invention.

### EXAMPLES

Materials and Methods

Oligonucleotides were synthesized by Invitrogen (Carlsbad, Calif.) and DNA sequencing was done by Genewiz Inc, South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). HER2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-HER2/neu antibody was purchased from Sigma.

Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N HER2/neu transgenic mice, which overexpress the rat HER2/neu onco-protein were housed and bred at the animal core facility at the University of Pennsylvania. The NT-2 tumor cell line expresses high levels of rat HER2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, MN) and was grown in complete C-RPMI medium. Bioluminescent work was conducted under guidance by the Small Animal Imaging Facility (SAIF) at the University of Pennsylvania (Philadelphia, Pa.).

Listeria Constructs and Antigen Expression

HER2/neu-pGEM7Z was kindly provided by Dr. Mark Greene at the University of Pennsylvania and contained the full-length human HER2/neu (hHer2) gene cloned into the pGEM7Z plasmid (Promega, Madison WI). This plasmid was used as a template to amplify three segments of hHER2/neu, namely, EC1, EC2, and IC1, by PCR using pfx DNA polymerase (Invitrogen) and the oligos indicated in Table 1.

TABLE 1
Primers for cloning of Human HER2-Chimera

| | | | Amino |
|---|---|---|---|
| | | | acid region |
| | | Base pair | or |
| | DNA sequence | region | junctions |
| HER2- | TGATCTCGAGACCCACCTGGACATGCTC (SEQ ID NO: | 120-510 | 40-170 |
| Chimera (F) | 57) | | |
| HerEC1- | CTACCAGGACACGATTTTGTGGAAG-AATATCCA | 510/1077 | 170/359 |
| EC2F | GGAGTTTGCTGGCTGC (SEQ ID NO: 58) | | |
| (Junction) | | | |
| HerEC1- | GCAGCCAGCAAACTCCTGGATATT-CTTCCACAA | | |
| EC2R | AATCGTGTCCTGGTAG (SEQ ID NO: 59) | | |
| (Junction) | | | |
| HerEC2- | CTGCCACCAGCTGTGCGCCCGAGGG- | 1554/2034 | 518/679 |
| ICIF | CAGCAGAAGATCCGGAAGTACACGA (SEQ ID NO: 60) | | |
| (Junction) | | | |
| HerEC2- | TCGTGTACTTCCGGATCTTCTGCTG | | |
| ICIR | CCCTCGGGC GCACAGCTGGTGGCAG (SEQ ID NO: 61) | | |
| (Junction) | | | |
| HER2- | GTGGCCCGGGTCTAGATTAGTCTAAGAGGCAGCCAT | 2034-2424 | 679-808 |
| Chimera (R) | AGG (SEQ ID NO: 62) | | |

The HER2/neu chimera construct was generated by direct fusion by the SOEing PCR method and each separate hHER2/neu segment as templates. Primers are shown in Table 2.

Sequence of primers for amplification of different
segments human Her2 regions

| | | Base pair | Amino acid |
|---|---|---|---|
| | DNA sequence | region | region |
| HER2-EC1(F) | CCGCCTCGAGGCCGCGAGCACCCAAGTG | 58-979 | 20-326 |
| | (SEQ ID NO: 63) | | |
| HER2- | CGCGACTAGTTTAATCCTCTGCTGTCACCT | | |
| EC1(R) | C (SEQ ID NO: 64) | | |
| HER2-EC2(F) | CCGCCTCGAGTACCTTTCTACGGACGTG | 907-1504 | 303-501 |
| | (SEQ ID NO: 65) | | |
| Her-2- | CGCGACTAGTTTACTCTGGCCGGTTGGCA | | |
| EC2(R) | G (SEQ ID NO: 66) | | |
| HER2-HER2- | CCGCCTCGAGCAGCAGAAGATCCGGAAGT | 2034-3243 | 679-1081 |
| IC1(F) | AC (SEQ ID NO: 67) | | |
| HER2-IC1(R) | CGCGACTAGTTTAAGCCCCTTCGGAGGGT | | |
| | G (SEQ ID NO: 68) | | |

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 μg/ml). In some experiments similar Listeria strains expressing hHER2/neu (Lm-hHER2) fragments were used for comparative purposes. These have been previously described. In all studies, an irrelevant Listeria construct (Lm-control) was included to account for the antigen independent effects of Listeria on the immune system. Lm-controls were based on the same Listeria platform as ADXS31-164, but expressed a different antigen such as HPV16-E7 or NY-ESO-1. Expression and secretion of fusion proteins from Listeria were tested. Each construct was passaged twice in vivo.

Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naive animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naive. Splenocytes from FVB/N mice were isolated one week after the last immunization and co-cultured in 24 well plates at 5×10⁶ cells/well in the presence of mitomycin C treated NT-2 cells in C-RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-Δ) using mouse IFN-γ Enzyme-linked immunosorbent assay (ELISA) kit according to manufacturer's recommendations.

INF-γ ELISpot Assay

Cryopreserved PBMC from each indicated time point were thawed, rested overnight at 37° C. and then counted. Cells were stimulated with 2.5 uM pools of overlapping human HER2/Neu peptides (11mers overlapping by 5 amino acids) that represent the EC1, EC2 and IC1 domains of HER2/Neu present in the chimeric vaccine, and recombinant human IL-2 (Invitrogen, Fredrick, MD) for 5 days. Cells were harvested, washed twice in 1× PBS and counted. IFN-γ ELISpot assays were performed according to the manufacturer's protocol using a commercial canine IFN-γ ELISpot assay kit (R&D Systems, Minneapolis, Minn.). Briefly, 0.8-2×105 stimulated cells were incubated with 2.5 uM of EC1, EC2 or IC1 peptide pools plus IL-2 or IL-2 alone (to determine background counts). All assays were performed in duplicates. Plates were developed according to the manufacturer's instructions. Spots were counted using a CTL-Immunospot analyzer (C.T.L, Shaker Heights, OH). Number of spots were normalized by subtracting twice the number of spots counted in non-stimulated wells.

Tumor Studies in Her2 Transgenic Animals

Six weeks old FVB/N rat HER2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNAlater at −20° C. In order to determine the effect of mutations in the HER2/neu protein on the escape of these tumors, genomic DNA was extracted using a genomic DNA isolation kit, and sequenced.

Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FB S. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat HER2/neu transgenic studies we used 8-14 mice per group, for all wild-type FVB/N studies we used at least 8 mice per group unless otherwise stated. All studies were repeated at least once except for the long term tumor study in HER2/neu transgenic mouse model.

### Example 1

### Generation of L. Monocytogenes Strains that Secrete LLO Fragments Fused to Her-2 Fragments: Construction of ADXS31-164

Construction of the chimeric HER2/neu gene (ChHer2) was described previously. Briefly, ChHer2 gene was generated by direct fusion of two extracellular (aa 40-170 and aa 359-433) and one intracellular fragment (aa 678-808) of the HER2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 1A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria vaccine strains. 2) Unlike pAdv138, pAdv164 does not harbor a copy of the prfA gene in the plasmid (see sequence below and FIG. 1A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 1B) as a band of ˜104 KD was detected by an anti-LLO antibody using Western Blot analysis. The Listeria backbone strain expressing only tLLO was used as negative control.

pAdv164 sequence (7075 base pairs) (see FIG. 1):

(SED ID NO: 53)

cggagtgtatactggcttactatgttggcactgatgagggtgtcagtgaa
gtgcttcatgtggcaggagaaaaaaggctgcaccggtgcgtcagcagaat
atgtgatacaggatatattccgcttcctcgctcactgactcgctacgctc
ggtcgttcgactgcggcgagcggaaatggcttacgaacggggcggagatt
tcctggaagatgccaggaagatacttaacagggaagtgagagggccgcgg
caaagccgtttttccataggctccgcccccctgacaagcatcacgaaatc
tgacgctcaaatcagtggtggcgaaacccgacaggactataaagatacca
ggcgtttccccctggcggctccctcgtgcgctctcctgttcctgcctttc
ggtttaccggtgtcattccgctgttatggccgcgtttgtctcattccacg
cctgacactcagttccgggtaggcagttcgctccaagctggactgtatgc
acgaaccccccgttcagtccgaccgctgcgccttatccggtaactatcgt
cttgagtccaacccggaaagacatgcaaaagcaccactggcagcagccac
tggtaattgatttagaggagttagtcttgaagtcatgcgccggttaaggc
taaactgaaaggacaagttttggtgactgcgctcctccaagccagttacc
tcggttcaaagagttggtagctcagagaaccttcgaaaaaccgccctgca
aggcggttttttcgttttcagagcaagagattacgcgcagaccaaaacga
tctcaagaagatcatcttattaatcagataaaatatttctagccctcctt
tgattagtatattcctatcttaaagttacttttatgtggaggcattaaca
tttgttaatgacgtcaaaaggatagcaagactagaataaagctataaagc
aagcatataatattgcgtttcatctttagaagcgaatttcgccaatatta
taattatcaaaagagaggggtggcaaacggtatttggcattattaggtta
aaaaatgtagaaggagagtgaaacccatgaaaaaaataatgctagttttt
attacacttatattagttagtctaccaattgcgcaacaaactgaagcaaa
ggatgcatctgcattcaataaagaaaattcaatttcatccatggcaccac
cagcatctccgcctgcaagtcctaagacgccaatcgaaaagaaacacgcg
gatgaaatcgataagtatatacaaggattggattacaataaaaacaatgt
attagtataccacggagatgcagtgacaaatgtgccgccaagaaaaggtt
acaaagatggaaatgaatatattgttgtggagaaaaagaagaaatccatc
aatcaaaataatgcagacattcaagttgtgaatgcaatttcgagcctaac
ctatccaggtgctctcgtaaaagcgaattcggaattagtagaaaatcaac
cagatgttctccctgtaaaacgtgattcattaacactcagcattgatttg
ccaggtatgactaatcaagacaataaaatagttgtaaaaaatgccactaa
atcaaacgttaacaacgcagtaaatacattagtggaaagatggaatgaaa
aatatgctcaagcttatccaaatgtaagtgcaaaaattgattatgatgac
gaaatggcttacagtgaatcacaattaattgcgaaatttggtacagcatt
taaagctgtaaataatagcttgaatgtaaacttcggcgcaatcagtgaag
ggaaaatgcaagaagaagtcattagttttaaacaaatttactataacgtg
aatgttaatgaacctacaagaccttccagatttttcggcaaagctgttac
taaagagcagttgcaagcgcttggagtgaatgcagaaaatcctcctgcat
atatctcaagtgtggcgtatggccgtcaagtttatttgaaattatcaact
aattcccatagtactaaagtaaaagctgcttttgatgctgccgtaagcgg
aaaatctgtctcaggtgatgtagaactaacaaatatcatcaaaaattctt
ccttcaaagccgtaatttacggaggttccgcaaaagatgaagttcaaatc
atcgacggcaacctcggagacttacgcgatattttgaaaaaaggcgctac
ttttaatcgagaaacaccaggagttcccattgcttatacaacaaacttcc
taaaagacaatgaattagctgttattaaaaacaactcagaatatattgaa
acaacttcaaaagcttatacagatggaaaaattaacatcgatcactctgg
aggatacgttgctcaattcaacatttcttgggatgaagtaaattatgatc
tcgagacccacctggacatgctccgccacctctaccagggctgccaggtg
gtgcagggaaacctggaactcacctacctgcccaccaatgccagcctgtc
cttcctgcaggatatccaggaggtgcagggctacgtgctcatcgctcaca
accaagtgaggcaggtcccactgcagaggctgcggattgtgcgaggcacc
cagctctttgaggacaactatgccctggccgtgctagacaatggagaccc
gctgaacaataccacccctgtcacaggggcctccccaggaggcctgcggg
agctgcagcttcgaagcctcacagagatcttgaaaggaggggtcttgatc
cagcggaacccccagctctgctaccaggacacgattttgtggaagaatat
ccaggagtttgctggctgcaagaagatctttgggagcctggcatttctgc
cggagagctttgatggggacccagcctccaacactgccccgctccagcca
gagcagctccaagtgtttgagactctggaagagatcacaggttacctata
catctcagcatggccggacagcctgcctgacctcagcgtcttccagaacc
tgcaagtaatccggggacgaattctgcacaatggcgcctactcgctgacc
ctgcaagggctgggcatcagctggctggggctgcgctcactgagggaact
gggcagtggactggccctcatccaccataacacccacctctgcttcgtgc
acacggtgccctgggaccagctctttcggaacccgcaccaagctctgctc
cacactgccaaccggccagaggacgagtgtgtgggcgagggcctggcctg
ccaccagctgtgcgcccgagggcagcagaagatccggaagtacacgatgc
ggagactgctgcaggaaacggagctggtggagccgctgacacctagcgga
gcgatgcccaaccaggcgcagatgcggatcctgaaagagacggagctgag
gaaggtgaaggtgcttggatctggcgcttttggcacagtctacaagggca
tctggatccctgatggggagaatgtgaaaattccagtggccatcaaagtg
ttgagggaaaacacatcccccaaagccaacaaagaaatcttagacgaagc
atacgtgatggctggtgtgggctccccatatgtctcccgccttctgggca
tctgcctgacatccacggtgcagctggtgacacagcttatgccctatggc
tgcctcttagactaatctagacccgggccactaactcaacgctagtagtg
gatttaatcccaaatgagccaacagaaccagaaccagaaacagaacaagt
aacattggagttagaaatggaagaagaaaaaagcaatgatttcgtgtgaa
taatgcacgaaatcattgcttatttttttaaaaagcgatatactagatat
aacgaaacaacgaactgaataaagaatacaaaaaaagagccacgaccagt
taaagcctgagaaactttaactgcgagccttaattgattaccaccaatca
attaaagaagtcgagacccaaaatttggtaaagtatttaattactttatt
aatcagatacttaaatatctgtaaacccattatatcgggtttttgagggg
atttcaagtctttaagaagataccaggcaatcaattaagaaaaacttagt
tgattgccttttttgttgtgattcaactttgatcgtagcttctaactaat
taattttcgtaagaaaggagaacagctgaatgaatatcccttttgttgta
gaaactgtgcttcatgacggcttgttaaagtacaaatttaaaaatagtaa
aattcgctcaatcactaccaagccaggtaaaagtaaaggggctatttttg
cgtatcgctcaaaaaaaagcatgattggcggacgtggcgttgttctgact
tccgaagaagcgattcacgaaaatcaagatacatttacgcattggacacc
aaacgtttatcgttatggtacgtatgcagacgaaaaccgttcatacacta
aaggacattctgaaaacaatttaagacaaatcaataccttctttattgat
tttgatattcacacggaaaaagaaactatttcagcaagcgatattttaac
aacagctattgatttaggttttatgcctacgttaattatcaaatctgata
aaggttatcaagcatattttgttttagaaacgccagtctatgtgacttca
aaatcagaatttaaatctgtcaaagcagccaaaataatctcgcaaaatat
ccgagaatattttggaaagtctttgccagttgatctaacgtgcaatcatt
ttgggattgctcgtataccaagaacggacaatgtagaattttttgatccc
aattaccgttattctttcaaagaatggcaagattggtctttcaaacaaac
agataataagggctttactcgttcaagtctaacggttttaagcggtacag
aaggcaaaaaacaagtagatgaaccctggtttaatctcttattgcacgaa
acgaaattttcaggagaaaagggtttagtagggcgcaatagcgttatgtt
taccctctctttagcctactttagttcaggctattcaatcgaaacgtgcg
aatataatatgtttgagtttaataatcgattagatcaacccttagaagaa
aaagaagtaatcaaaattgttagaagtgcctattcagaaaactatcaagg
ggctaatagggaatacattaccattctttgcaaagcttgggtatcaagtg
atttaaccagtaaagatttatttgtccgtcaagggtggtttaaattcaag
aaaaaaagaagcgaacgtcaacgtgttcatttgtcagaatggaaagaaga
tttaatggcttatattagcgaaaaaagcgatgtatacaagccttatttag
cgacgaccaaaaaagagattagagaagtgctaggcattcctgaacggaca
ttagataaattgctgaaggtactgaaggcgaatcaggaaattttctttaa
gattaaaccaggaagaaatggtggcattcaacttgctagtgttaaatcat
tgttgctatcgatcattaaattaaaaaaagaagaacgagaaagctatata
aaggcgctgacagcttcgtttaatttagaacgtacatttattcaagaaac
tctaaacaaattggcagaacgccccaaaacggacccacaactcgatttgt
ttagctacgatacaggctgaaaataaaacccgcactatgccattacattt
atatctatgatacgtgtttgtttttctttgctggctagcttaattgctta
tatttacctgcaataaaggatttcttacttccattatactcccattttcc
aaaaacatacggggaacacgggaacttattgtacaggccacctcatagtt
aatggtttcgagccttcctgcaatctcatccatggaaatatattcatccc
cctgccggcctattaatgtgacttttgtgcccggcggatattcctgatcc
agctccaccataaattggtccatgcaaattcggccggcaattttcaggcg
ttttcccttcacaaggatgtcggtccctttcaattttcggagccagccgt
ccgcatagcctacaggcaccgtcccgatccatgtgtctttttccgctgtg
tactcggctccgtagctgacgctctcgccttttctgatcagtttgacatg
tgacagtgtcgaatgcagggtaaatgccggacgcagctgaaacggtatct
cgtccgacatgtcagcagacgggcgaaggccatacatgccgatgccgaat
ctgactgcattaaaaaagccttttttcagccggagtccagcggcgctgtt
cgcgcagtggaccattagattctttaacggcagcggagcaatcagctctt
taaagcgctcaaactgcattaagaaatagcctctttctttttcatccgct
gtcgcaaaatgggtaaatacccctttgcactttaaacgagggttgcggtc
aagaattgccatcacgttctgaacttcttcctctgtttttacaccaagtc
tgttcatccccgtatcgaccttcagatgaaaatgaagagaaccttttttc
gtgtggcgggctgcctcctgaagccattcaacagaataacctgttaaggt
cacgtcatactcagcagcgattgccacatactccgggggaaccgcgccaa
gcaccaatataggcgccttcaatccctttttgcgcagtgaaatcgcttca
tccaaaatggccacggccaagcatgaagcacctgcgtcaagagcagcctt
tgctgtttctgcatcaccatgcccgtaggcgtttgctttcacaactgcca
tcaagtggacatgttcaccgatatgttttttcatattgctgacattttcc
tttatcgcggacaagtcaatttccgcccacgtatctctgtaaaaaggttt
tgtgctcatggaaaactcctctcttttttcagaaaatcccagtacgtaat
taagtatttgagaattaattttatattgattaatactaagtttacccagt
tttcacctaaaaaacaaatgatgagataatagctccaaaggctaaagagg
actataccaactatttgttaattaa

### Example 2

### ADXS31-164 is as Immunogenic as LM-LLO-ChHER2

Immunogenic properties of ADXS31-164 in generating anti-HER2/neu specific cytotoxic T cells were compared to those of the Lm-LLO-ChHer2 vaccine in a standard CTL assay. Both vaccines elicited strong but comparable cytotoxic T cell responses toward HER2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria vaccine (FIG. 2A). ADXS31-164 was also able to stimulate the secretion of IFN-γ by the splenocytes from wild type FVB/N mice (FIG. 2B). This was detected in the culture supernatants of these cells that were co-cultured with mitomycin C treated NT-2 cells, which express high levels of HER2/neu antigen (FIG. 5C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 11 or KIFGSLAFL SEQ ID NO: 12) or intracellular (RLLQETELV SEQ ID NO: 13) domains of the HER2/neu molecule (FIG. 2C). A recombinant ChHer2 protein was used as positive control and an irrelevant peptide or no peptide as negative controls. The data from this experiment show that ADXS31-164 is able to elicit anti-HER2/neu specific immune responses to human epitopes that are located at different domains of the targeted antigen.

### Example 3

### ADXS31-164 was more Efficacious than LM-LLO-ChHER2 in Preventing the Onset of Spontaneous Mammary Tumors

Anti-tumor effects of ADXS31-164 were compared to those of Lm-LLO-ChHer2 in HER2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Liseria-HER2/neu recombinant vaccines caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 3). These results indicate that despite being more attenuated, ADXS31-164 is more efficacious than Lm-LLO-ChHer2 in preventing the onset of spontaneous mammary tumors in HER2/neu transgenic animals.

### Example 4

### Mutations in HER2/NEU Gene upon Immunization with ADXS31-164

Mutations in the MHC class I epitopes of HER2/neu have been considered responsible for tumor escape upon immunization with small fragment vaccines or trastuzumab (Herceptin), a monoclonal antibody that targets an epitope in the extracellular domain of HER2/neu. To assess this, genomic material was extracted from the escaped tumors in the transgenic animals and sequenced the corresponding fragments of the neu gene in tumors immunized with the chimeric or control vaccines. Mutations were not observed within the HER2/neu gene of any vaccinated tumor samples suggesting alternative escape mechanisms (data not shown).

### Example 5

### ADXS31-164 causes a Significant Decrease in Intra-Tumoral T Regulatory Cells

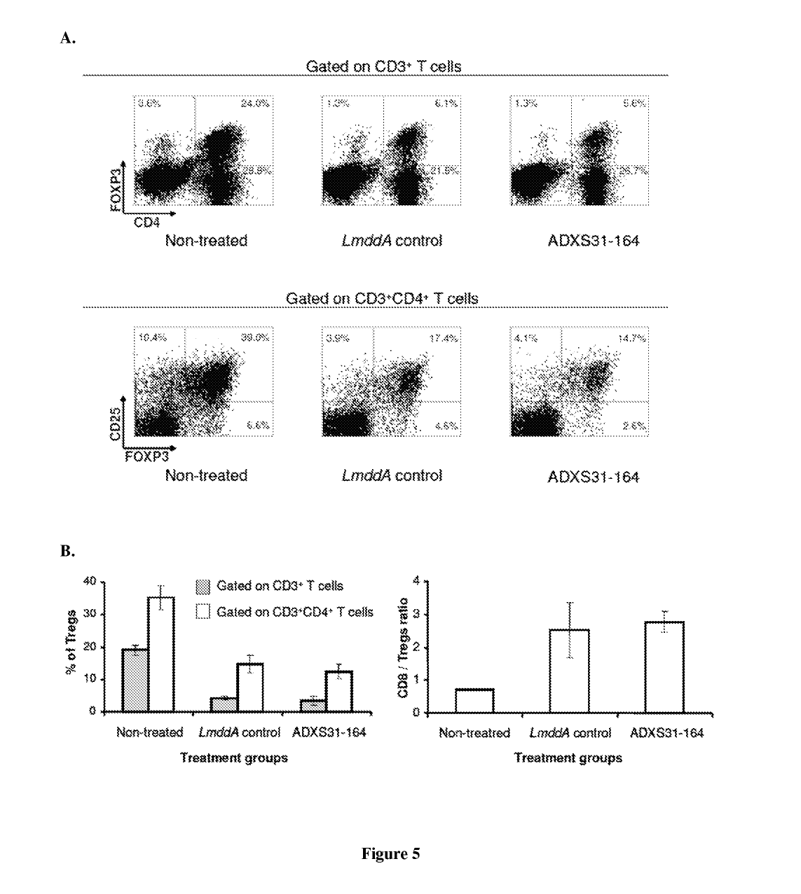

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naïve animals (See FIG. 4). In contrast, immunization with the Listeria vaccines caused a considerable impact on the presence of Tregs in the tumors (FIG. 5A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 5B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 6

### No Escape Mutations were Introduced by Listeria Vaccine Expressing HER-2 Chimera

Tumor samples of the mice immunized with different vaccines such as Lm-LLO-138, LmddA164 and irrelevant vaccine Lm-LLO-NY were harvested. The DNA was purified from these samples and the DNA fragments corresponding to HER2/neu regions ICL EC1 and EC2 were amplified and were sequenced to determine if there were any immune escape mutations. The alignment of sequence from each DNA was performed using CLUSTALW. The results of the analysis indicated that there were no mutations in the DNA sequences harvested from tumors. The detailed analysis of these sequences is shown below.

Alignment of EC2 (975-1029 by of HER2/Neu)

Reference

(SEQ ID NO: 14)

GGTCACAGCTGAGGACGGAACACAGCGTTGTGAGAAATGCAGCAAGCCCT
GTGCT
Lm-LLO-138-2
GGTCACAGCTGAGGACGGAACACAGCGTTGTGAGAAATGCAGCAAGCCCT
GTGCT
Lm-LLO-138-3
GGTCACAGCTGAGGACGGAACACAGCGTTGTGAGAAATGCAGCAAGCCCT
GTGCT
Lm-ddA-164-1
GGTCACAGCTGAGGACGGAACACAGCGTTGTGAGAAATGCAGCAAGCCCT
GTGCT
LmddA164-2
GGTCACAGCTGAGGACGGAACACAGCGTTGTGAGAAATGCAGCAAGCCCT
GTGCT
Lm-ddA-164-3
GGTCACAGCTGAGGACGGAACACAGCGTTGTGAGAAATGCAGCAAGCCCT
GTGCT
LmddA164-4
GGTCACAGCTGAGGACGGAACACAGCGTTGTGAGAAATGCAGCAAGCCCT
GTGCT
Lm-ddA-164-5
GGTCACAGCTGAGGACGGAACACAGCGTTGTGAGAAATGCAGCAAGCCCT
GTGCT
LmddA-164-6
GGTCACAGCTGAGGACGGAACACAGCGTTCTGAGAAATGCAGCAAGCCCT
GTGCT
Reference

(SEQ ID NO: 15)

CGAGTGTGCTATGGTCTGGGCATGGAGCACCTTCGAGGGGCGAGGGCCAT
CACCAGTGAC
Lm-LLO-138-2
CGAGTGTGCTATGGTCTGGGCATGGAGCACCTTCGAGGGGCGAGGGCCAT
CACCAGTGAC
Lm-LLO-138-3
CGAGTGTGCTATGGTCTGGGCATGGAGCACCTTCGAGGGGCGAGGGCCAT
CACCAGTGAC
Lm-ddA-164-1
CGAGTGTGCTATGGTCTGGGCATGGAGCACCTTCGAGGGGCGAGGGCCAT
CACCAGTGAC
LmddA164-2
CGAGTGTGCTATGGTCTGGGCATGGAGCACCTTCGAGGGGCGAGGGCCAT
CACCAGTGAC
Lm-ddA-164-3
CGAGTGTGCTATGGTCTGGGCATGGAGCACCTTCGAGGGGCGAGGGCCAT
CACCAGTGAC
LmddA164-4
CGAGTGTGCTATGGTCTGGGCATGGAGCACCTTCGAGGGGCGAGGGCCAT
CACCAGTGAC
Lm-ddA-164-5
CGAGTGTGCTATGGTCTGGGCATGGAGCACCTTCGAGGGGCGAGGGCCAT
CACCAGTGAC
LmddA-164-6
CGAGTGTGCTATGGTCTGGGCATGGAGCACCTTCGAGGGGCGAGGGCCAT
CACCAGTGAC
Reference

(SEQ ID No: 16)

AATGTCCAGGAGTTTGATGGCTGCAAGAAGATCTTTGGGAGCCTGGCATT
TTTGCCGGAG
Lm-LLO-138-2
AATGTCCAGGAGTTTGATGGCTGCAAGAAGATCTTTGGGAGCCTGGCATT
TTTGCCGGAG
Lm-LLO-138-3
AATGTCCAGGAGTTTGATGGCTGCAAGAAGATCTTTGGGAGCCTGGCATT
TTTGCCGGAG
Lm-ddA-164-1
AATGTCCAGGAGTTTGATGGCTGCAAGAAGATCTTTGGGAGCCTGGCATT
TTTGCCGGAG
LmddA164-2
AATGTCCAGGAGTTTGATGGCTGCAAGAAGATCTTTGGGAGCCTGGCATT
TTTGCCGGAG
Lm-ddA-164-3
AATGTCCAGGAGTTTGATGGCTGCAAGAAGATCTTTGGGAGCCTGGCATT
TTTGCCGGAG
LmddA164-4
AATGTCCAGGAGTTTGATGGCTGCAAGAAGATCTTTGGGAGCCTGGCATT
TTTGCCGGAG
Lm-ddA-164-5
AATGTCCAGGAGTTTGATGGCTGCAAGAAGATCTTTGGGAGCCTGGCATT
TTTGCCGGAG
LmddA-164-6
AATGTCCAGGAGTTTGATGGCTGCAAGAAGATCTTTGGGAGCCTGGCATT
TTTGCCGGAG
Reference

(SEQ ID NO: 17)

AGCTTTGATGGGGACCCCTCCTCCGGCATTGCTCCGCTGAGGCCTGAGCA
GCTCCAAGTG
Lm-LLO-138-2
AGCTTTGATGGGGACCCCTCCTCCGGCATTGCTCCGCTGAGGCCTGAGCA
GCTCCAAGTG
Lm-LLO-138-3
AGCTTTGATGGGGACCCCTCCTCCGGCATTGCTCCGCTGAGGCCTGAGCA
GCTCCAAGTG
Lm-ddA-164-1
AGCTTTGATGGGGACCCCTCCTCCGGCATTGCTCCGCTGAGGCCTGAGCA
GCTCCAAGTG
LmddA164-2
AGCTTTGATGGGGACCCCTCCTCCGGCATTGCTCCGCTGAGGCCTGAGCA
GCTCCAAGTG
Lm-ddA-164-3
AGCTTTGATGGGGACCCCTCCTCCGGCATTGCTCCGCTGAGGCCTGAGCA
GCTCCAAGTG
LmddA164-4
AGCTTTGATGGGGACCCCTCCTCCGGCATTGCTCCGCTGAGGCCTGAGCA
GCTCCAAGTG
Lm-ddA-164-5
AGCTTTGATGGGGACCCCTCCTCCGGCATTGCTCCGCTGAGGCCTGAGCA
GCTCCAAGTG
LmddA-164-6
AGCTTTGATGGGGACCCCTCCTCCGGCATTGCTCCGCTGAGGCCTGAGCA
GCTCCAAGTG
Reference

(SEQ ID NO: 18)

TTCGAAACCCTGGAGGAGATCACAGGTTACCTGTACATCTCAGCATGGCC
AGACAGTCTC
Lm-LLO-138-2
TTCGAAACCCTGGAGGAGATCACAGGTTACCTGTACATCTCAGCATGGCC
AGACAGTCTC
Lm-LLO-138-3
TTCGAAACCCTGGAGGAGATCACAGGTTACCTGTACATCTCAGCATGGCC
AGACAGTCTC
Lm-ddA-164-1
TTCGAAACCCTGGAGGAGATCACAGGTTACCTGTACATCTCAGCATGGCC
AGACAGTCTC
LmddA164-2
TTCGAAACCCTGGAGGAGATCACAGGTTACCTGTACATCTCAGCATGGCC
AGACAGTCTC
Lm-ddA-164-3
TTCGAAACCCTGGAGGAGATCACAGGTTACCTGTACATCTCAGCATGGCC
AGACAGTCTC
LmddA164-4
TTCGAAACCCTGGAGGAGATCACAGGTTACCTGTACATCTCAGCATGGCC
AGACAGTCTC
Lm-ddA-164-5
TTCGAAACCCTGGAGGAGATCACAGGTTACCTGTACATCTCAGCATGGCC
ANACAGTCTC
LmddA-164-6
TTCGAAACCCTGGAGGAGATCACAGGTTACCTGTACATCTCAGCATGGCC
AGACAGTCT
Reference

(SEQ ID NO: 19)

CGTGACCTCAGTGTCTTCCAGAACCTTCGAATCATTCGGGGACGGATTCT
CCACGATGGC
Lm-LLO-138-2
CGTGACCTCAGTGTCTTCCAGAACCTTCGAATCATTCGGGGACGGATTCT
CCACGATGGC
Lm-LLO-138-3
CGTGACCTCAGTGTCTTCCAGAACCTTCGAATCATTCGGGGACGGATTCT
CCACGATGGC
Lm-ddA-164-1
CGTGACCTCAGTGTCTTCCAGAACCTTCGAATCATTCGGGGACGGATTCT
CCACGATGGC
LmddA164-2
CGTGACCTCAGTGTCTTCCAGAACCTTCGAATCATTCGGGGACGGATTCT
CCACGATGGC
Lm-ddA-164-3
CGTGACCTCAGTGTCTTCCAGAACCTTCGAATCATTCGGGGACGGATTCT
CCACGATGGC
LmddA164-4
CGTGACCTCAGTGTCTTCCAAAACCTTCGAATCATTCGGGGACGGATTCT
CCACGATGGC
Lm-ddA-164-5
CGTGACCTCAGTGTCTTCCAAAACCTTCGAATCATTCGGGGACGGATTCT
CCACGATGGC
LmddA-164-6
CGTGACCTCAGTGTCTTCCAAAACCTTCGAATCATTCGGGGACGGATTCT
CCACGATGGC
Reference

(SEQ ID NO: 20)

GCGTACTCATTGACACTGCAAGGCCTGGGGATCCACTCGCTGGGGCTGCG
CTCACTGCGG
Lm-LLO-138-2
GCGTACTCATTGACACTGCAAGGCCTGGGGATCCACTCGCTGGGGCTGCG
CTCACTGCGG
Lm-LLO-138-3
GCGTACTCATTGACACTGCAAGGCCTGGGGATCCACTCGCTGGGGCTGCG
CTCACTGCGG
Lm-ddA-164-1
GCGTACTCATTGACACTGCAAGGCCTGGGGATCCACTCGCTGGGGCTGCG
CTCACTGCGG
LmddA164-3
GCGTACTCATTGACACTGCAAGGCCTGGGGATCCACTCGCTGGGGCTGCG
CTCACTGCGG
Lm-ddA-164-5
GCGTACTCATTGACACTGCAAGGCCTGGGGATCCACTCGCTGGGGCTGCG
CTCACTGCGG
Lm-ddA-164-6
GCGTACTCATTGACACTGCAAGGCCTGGGGATCCACTCGCTGGGGCTGCG
CTCACTGCGG
Reference

(SEQ ID NO: 21)

GAGCTGGGCAGTGGATTGGCTCTGATTCACCGCAACGCCCATCTCTGCTT
TGTACACACT
Lm-LLO-138-2
GAGCTGGGCAGTGGATTGGCTCTGATTCACCGCAACGCCCATCTCTGCTT
TGTACACACT
Lm-LLO-138-3
GAGCTGGGCAGTGGATTGGCTCTGATTCACCGCAACGCCCATCTCTGCTT
TGTACACACT
Lm-ddA-164-1
GAGCTGGGCAGTGGATTGGCTCTGATTCACCGCAACGCCCATCTCTGCTT
TGTACACACT
LmddA164-3
GAGCTGGGCAGTGGATTGGCTCTGATTCACCGCAACGCCCATCTCTGCTT
TGTACACACT
Lm-ddA-164-5
GAGCTGGGCAGTGGATTGGCTCTGATTCACCGCAACGCCCATCTCTGCTT
TGTACACACT
Lm-ddA-164-6
GAGCTGGGCAGTGGATTGGCTCTGATTCACCGCAACGCCCATCTCTGCTT
TGTACACACT
Reference

(SEQ ID NO: 22)

GTACCTTGGGACCAGCTCTTCCGGAACCCACATCAGGCCCTGCTCCACAG
TGGGAACCGG
Lm-LLO-138-2
GTACCTTGGGACCAGCTCTTCCGGAACCCACATCAGGCCCTGCTCCACAG
TGGGAACCGG
Lm-LLO-138-3
GTACCTTGGGACCAGCTCTTCCGGAACCCACATCAGGCCCTGCTCCACAG
TGGGAACCGG
Lm-ddA-164-1
GTACCTTGGGACCAGCTCTTCCGGAACCCACATCAGGCCCTGCTCCACAG
TGGGAACCGG
LmddA164-3
GTACCTTGGGACCAGCTCTTCCGGAACCCACATCAGGCCCTGCTCCACAG
TGGGAACCGG
Lm-ddA-164-5
GTACCTTGGGACCANCTCTTCCGGAACCCACATCAGGCCCTGCTCCACAG
TGGGAACCGG
Lm-ddA-164-6
GTACCTTGGGACCAGCTCTTCCGGAACCCACATCAGGCCCTGCTCCACAG
TGGGAACCGG
Reference

(SEQ ID NO: 23)

CCGGAAGAGGATTGTGGTCTCGAGGGCTTGGTCTGTAACTCACTGTGTGC
CCACGGGCAC
Lm-LLO-138-2
CCGGAAGAGGATTGTGGTCTCGAGGGCTTGGTCTGTAACTCACTGTGTGC
CCACGGGCAC
Lm-LLO-138-3
CCGGAAGAGGATTGTGGTCTCGAGGGCTTGGTCTGTAACTCACTGTGTGC
CCACGGGCAC
Lm-ddA-164-1
CCGGAAGAGGATTGTGGTCTCGAGGGCTTGGTCTGTAACTCACTGTGTGC
CCACGGGCAC
LmddA164-3
CCGGAAGAGGATTGTGGTCTCGAGGGCTTGGTCTGTAACTCACTGTGTGC
CCACGGGCAC
Lm-ddA-164-6
CCGGAAGAGGATTGTGGTCTCGAGGGCTTGGTCTGTAACTCACTGTGTGC
CCACGGGCAC
Reference

(SEQ ID NO: 24)

TGCTGGGGGCCAGGGCCCACCCAGTGTGTCAACTGCAGTCATTTCCTTCG
GGGCCAGGAG
Lm-LLO-138-2
TGCTGGGGGCCAGGGCCCACCCAGTGTGTCAACTGCAGTCATTTCCTTCG
GGGCCAGGAG
Lm-LLO-138-3
TGCTGGGGGCCAGGGCCCACCCAGTGTGTCAACTGCAGTCATTTCCTTCG
GGGCCAGGAG
Lm-ddA-164-1
TGCTGGGGGCCAGGGCCCACCCAGTGTGTCAACTGCAGTCATTTCCTTCG
GGGCCAGGAG
LmddA164-3
TGCTGGGGGCCAGGGCCCACCCAGTGTGTCAACTGCAGTCATTTCCTTCG
GGGCCAGGAG
Lm-ddA-164-6
TGCTGGGGGCCAGGGCCCACCCA---------------------------
----------

Alignment of IC1 (2114-3042 by of HER2/Neu)

Reference

(SEQ ID NO: 25)

CGCCCAGCGGAGCAATGCCCAACCAGGCTCAGATGCGGATCCTAAAAGAG
ACGGAGC
Lm-LLO-NY-2
CGCCCAGCGGAGCAATGCCCAACCAGGCTCAGATGCGGATCCTAAAAGAG
ACGGAGC
Lm-LLO-138-4
CGCCCAGCGGAGCAATGCCCAACCAGGCTCAGATGCGGATCCTAAAAGAG
ACGGAGC
Lm-ddA-164-2
CGCCCAGCGGAGCAATGCCCAACCAGGCTCAGATGCGGATCCTAAAAGAG
ACGGAGC
Lm-ddA-164-3
CGCCCAGCGGAGCAATGCCCAACCAGGCTCAGATGCGGATCCTAAAAGAG
ACGGAGC
Lm-ddA164-6
CGCCCAGCGGAGCAATGCCCAACCAGGCTCAGATGCGGATCCTAAAAGAG
ACGGAGC
Reference

(SEQ ID NO: 26)

TAAGGAAGGTGAAGGTGCTTGGATCAGGAGCTTTTGGCACTGTCTACAAG
GGCATCTGGA
Lm-LLO-NY-1
TAAGGAAGGTGAAGGTGCTTGGATCAGGAGCTTTTGGCACTGTCTACAAG
GGCATCTGGA
Lm-LLO-NY-2
TAAGGAAGGTGAAGGTGCTTGGATCAGGAGCTTTTGGCACTGTCTACAAG
GGCATCTGGA
Lm-LLO-138-1
TAAGGAAGGTGAACGTGCTTGGATCAGGAGCTTTTGGCACTGTCTACAAG
GGCATCTGGA
Lm-LLO-138-2
TAAGGAAGGTGAAGGTGCTTGGATCAGGAGCTTTTGGCACTGTCTACAAG
GGCATCTGGA
Lm-LLO-138-3
TAAGGAAGGTGAAGGTGCTTGGATCAGGAGCTTTTGGCACTGTCTACAAG
GGCATCTGGA
Lm-LLO-138-4
TAAGGAAGGTGAAGGTGCTTGGATCAGGAGCTTTTGGCACTGTCTACAAG
GGCATCTGGA
Lm-ddA-164-1
TAAGGAAGGTGAAGGTGCTTGGATCAGGAGCTTTTGGCACTGTCTACAAG
GGCATCTGGA
Lm-ddA-164-2
TAAGGAAGGTGAAGGTGCTTGGATCAGGAGCTTTTGGCACTGTCTACAAG
GGCATCTGGA
Lm-ddA-164-3
TAAGGAAGGTGAAGGTGCTTGGATCAGGAGCTTTTGGCACTGTCTACAAG
GGCATCTGGA
Lm-ddA-164-4
TAAGGAAGGTGAAGGTGCTTGGATCAGGAGCTTTTGGCACTGTCTACAAG
GGCATCTGGA
Lm-ddA-164-5
TAAGGAAGGTGAAGGTGCTTGGATCAGGAGCTTTTGGCACTGTCTACAAG
GGCATCTGGA
Lm-ddA164-6
TAAGGAAGGTGAAGGTGCTTGGATCAGGAGCTTTTGGCACTGTCTACAAG
GGCATCTGGA
Reference

(SEQ ID NO: 27)

TCCCAGATGGGGAGAATGTGAAAATCCCCGTGGCTATCAAGGTGTTGAGA
GAAAACACAT
Lm-LLO-NY-1
TCCCAGATGGGGAGAATGTGAAAATCCCCGTGGCTATCAAGGTGTTGAGA
GAAAACACAT
Lm-LLO-NY-2
TCCCAGATGGGGAGAATGTGAAAATCCCCGTGGCTATCAAGGTGTTGAGA
GAAAACACAT
Lm-LLO-138-1
TCCCAGATGGGGAGAATGTGAAAATCCCCGTGGCTATCAAGGTGTTGAGA
GAAAACACAT
Lm-LLO-138-2
TCCCAGATGGGGAGAATGTGAAAATCCCCGTGGCTATCAAGGTGTTGAGA
GAAAACACAT
Lm-LLO-138-3
TCCCAGATGGGGAGAATGTGAAAATCCCCGTGGCTATCAAGGTGTTGAGA
GAAAACACAT
Lm-LLO-138-4
TCCCAGATGGGGAGAATGTGAAAATCCCCGTGGCTATCAAGGTGTTGAGA
GAAAACACAT
Lm-ddA-164-1
TCCCAGATGGGGAGAATGTGAAAATCCCCGTGGCTATCAAGGTGTTGAGA
GAAAACACAT
Lm-ddA-164-2
TCCCAGATGGGGAGAATGTGAAAATCCCCGTGGCTATCAAGGTGTTGAGA
GAAAACACAT
Lm-ddA-164-3
TCCCAGATGGGGAGAATGTGAAAATCCCCGTGGCTATCAAGGTGTTGAGA
GAAAACACAT
Lm-ddA-164-4
TCCCAGATGGGGAGAATGTGAAAATCCCCGTGGCTATCAAGGTGTTGAGA
GAAAACACAT
Lm-ddA-164-5
TCCCAGATGGGGAGAATGTGAAAATCCCCGTGGCTATCAAGGTGTTGAGA
GAAAACACAT
Lm-ddA164-6
TCCCAGATGGGGAGAATGTGAAAATCCCCGTGGCTATCAAGGTGTTGAGA
GAAAACACAT
Reference

(SEQ ID NO: 28)

CTCCTAAAGCCAACAAAGAAATTCTAGATGAAGCGTATGTGATGGCTGGT
GTGGGTTCTC
Lm-LLO-NY-1
CTCCTAAAGCCAACAAAGAAATTCTAGATGAAGCGTATGTGATGGCTGGT
GTGGGTTCTC
Lm-LLO-NY-2
CTCCTAAAGCCAACAAAGAAATTCTAGATGAAGCGTATGTGATGGCTGGT
GTGGGTTCTC
Lm-LLO-138-1
CTCCTAAAGCCAACAAAGAAATTCTAGATGAAGCGTATGTGATGGCTGGT
GTGGGTTCTC
Lm-LLO-138-2
CTCCTAAAGCCAACAAAGAAATTCTAGATGAAGCGTATGTGATGGCTGGT
GTGGGTTCTC
Lm-LLO-138-3
CTCCTAAAGCCAACAAAGAAATTCTAGATGAAGCGTATGTGATGGCTGGT
GTGGGTTCTC
lm-LLO-138-4
CTCCTAAAGCCAACAAAGAAATTCTAGATGAAGCGTATGTGATGGCTGGT
GTGGGTTCTC
Lm-ddA-164-1
CTCCTAAAGCCAACAAAGAAATTCTAGATGAAGCGTATGTGATGGCTGGT
GTGGGTTCTC
Lm-ddA-164-2
CTCCTAAAGCCAACAAAGAAATTCTAGATGAAGCGTATGTGATGGCTGGT
GTGGGTTCTC
Lm-ddA-164-3
CTCCTAAAGCCAACAAAGAAATTCTAGATGAAGCGTATGTGATGGCTGGT
GTGGGTTCTC
Lm-ddA-164-4
CTCCTAAAGCCAACAAAGAAATTCTAGATGAAGCGTATGTGATGGCTGGT
GTGGGTTCTC
Lm-ddA-164-5
CTCCTAAAGCCAACAAAGAAATTCTAGATGAAGCGTATGTGATGGCTGGT
GTGGGTTCTC
Lm-ddA164-6
CTCCTAAAGCCAACAAAGAAATTCTAGATGAAGCGTATGTGATGGCTGGT
GTGGGTTCTC
Reference

(SEQ ID NO: 29)

CGTATGTGTCCCGCCTCCTGGGCATCTGCCTGACATCCACAGTACAGCTG
GTGACACAGC
Lm-LLO-NY-1
CGTATGTGTCCCGCCTCCTGGGCATCTGCCTGACATCCACAGTACAGCTG
GTGACACAGC
Lm-LLO-NY-2
CGTATGTGTCCCGCCTCCTGGGCATCTGCCTGACATCCACAGTACAGCTG
GTGACACAGC
Lm-LLO-138-1
CGTATGTGTCCCGCCTCCTGGGCATCTGCCTGACATCCACAGTACAGCTG
GTGACACAGC
Lm-LLO-138-2
CGTATGTGTCCCGCCTCCTGGGCATCTGCCTGACATCCACAGTACAGCTG
GTGACACAGC
Lm-LLO-138-3
CGTATGTGTCCCGCCTCCTGGGCATCTGCCTGACATCCACAGTACAGCTG
GTGACACAGC
Lm-LLO-138-4
CGTATGTGTCCCGCCTCCTGGGCATCTGCCTGACATCCACAGTACAGCTG
GTGACACAGC
Lm-ddA-164-1
CGTATGTGTCCCGCCTCCTGGGCATCTGCCTGACATCCACAGTACAGCTG
GTGACACAGC
Lm-ddA-164-2
CGTATGTGTCCCGCCTCCTGGGCATCTGCCTGACATCCACAGTACAGCTG
GTGACACAGC
Lm-ddA-164-3
CGTATGTGTCCCGCCTCCTGGGCATCTGCCTGACATCCACAGTACAGCTG
GTGACACAGC
Lm-ddA-164-4
CGTATGTGTCCCGCCTCCTGGGCATCTGCCTGACATCCACAGTACAGCTG
GTGACACAGC
Lm-ddA-164-5
CGTATGTGTCCCGCCTCCTGGGCATCTGCCTGACATCCACAGTACAGCTG
GTGACACAGC
Lm-ddA164-6
CGTATGTGTCCCGCCTCCTGGGCATCTGCCTGACATCCACAGTACAGCTG
GTGACACAGC
Reference

(SEQ ID NO: 30)

TTATGCCCTACGGCTGCCTTCTGGACCATGTCCGAGAACACCGAGGTCGC
CTAGGCTCCC
Lm-LLO-NY-1
TTATGCCCTACGGCTGCCTTCTGGACCATGTCCGAGAACACCGAGGTCGC
CTAGGCTCCC
Lm-LLO-NY-2
TTATGCCCTACGGCTGCCTTCTGGACCATGTCCGAGAACACCGAGGTCGC
CTAGGCTCCC
Lm-LLO-138-1
TTATGCCCTACGGCTGCCTTCTGGACCATGTCCGAGAACACCGAGGTCGC
CTAGGCTCCC
Lm-LLO-138-2
TTATGCCCTACGGCTGCCTTCTGGACCATGTCCGAGAACACCGAGGTCGC
CTAGGCTCCC
Lm-LLO-138-3
TTATGCCCTACGGCTGCCTTCTGGACCATGTCCGAGAACACCGAGGTCGC
CTAGGCTCCC
Lm-LLO-138-4
TTATGCCCTACGGCTGCCTTCTGGACCATGTCCGAGAACACCGAGGTCGC
CTAGGCTCCC
Lm-ddA-164-1
TTATGCCCTACGGCTGCCTTCTGGACCATGTCCGAGAACACCGAGGTCGC
CTAGGCTCCC
Lm-ddA-164-2
TTATGCCCTACGGCTGCCTTCTGGACCATGTCCGAGAACACCGAGGTCGC
CTAGGCTCCC
Lm-ddA-164-3
TTATGCCCTACGGCTGCCTTCTGGACCATGTCCGAGAACACCGAGGTCGC
CTAGGCTCCC
Lm-ddA-164-4
TTATGCCCTACGGCTGCCTTCTGGACCATGTCCGAGAACACCGAGGTCGC
CTAGGCTCCC
Lm-ddA-164-5
TTATGCCCTACGGCTGCCTTCTGGACCATGTCCGAGAACACCGAGGTCGC
CTAGGCTCCC
Lm-ddA164-6
TTATGCCCTACGGCTGCCTTCTGGACCATGTCCGAGAACACCGAGGTCGC
CTAGGCTCCC
Reference

(SEQ ID NO: 31)

AGGACCTGCTCAACTGGTGTGTTCAGATTGCCAAGGGGATGAGCTACCTG
GAGGACGTGC
Lm-LLO-NY-1
AGGACCTGCTCAACTGGTGTGTTCAGATTGCCAAGGGGATGAGCTACCTG
GAGGACGTGC
Lm-LLO-NY-2
AGGACCTGCTCAACTGGTGTGTTCAGATTGCCAAGGGGATGAGCTACCTG
GAGGACGTGC
Lm-LLO-138-1
AGGACCTGCTCAACTGGTGTGTTCAGATTGCCAAGGGGATGAGCTACCTG
GAGGACGTGC
Lm-LLO-138-2
AGGACCTGCTCAACTGGTGTGTTCAGATTGCCAAGGGGATGAGCTACCTG
GAGGACGTGC
Lm-LLO-138-3
AGGACCTGCTCAACTGGTGTGTTCAGATTGCCAAGGGGATGAGCTACCTG
GAGGACGTGC
Lm-LLO-138-4
AGGACCTGCTCAACTGGTGTGTTCAGATTGCCAAGGGGATGAGCTACCTG
GAGGACGTGC
Lm-ddA-164-1
AGGACCTGCTCAACTGGTGTGTTCAGATTGCCAAGGGGATGAGCTACCTG
GAGGACGTGC
Lm-ddA-164-2
AGGACCTGCTCAACTGGTGTGTTCAGATTGCCAAGGGGATGAGCTACCTG
GAGGACGTGC
Lm-ddA-164-3
AGGACCTGCTCAACTGGTGTGTTCAGATTGCCAAGGGGATGAGCTACCTG
GAGGACGTGC
Lm-ddA-164-4
AGGACCTGCTCAACTGGTGTGTTCAGATTGCCAAGGGGATGAGCTACCTG
GAGGACGTGC
Lm-ddA-164-5
AGGACCTGCTCAACTGGTGTGTTCAGATTGCCAAGGGGATGAGCTACCTG
GAGGACGTGC
Lm-ddA164-6
AGGACCTGCTCAACTGGTGTGTTCAGATTGCCAAGGGGATGAGCTACCTG
GAGGACGTGC
Reference

(SEQ ID NO: 32)

GGCTTGTACACAGGGACCTGGCTGCCCGGAATGTGCTAGTCAAGAGTCCC
AACCACGTCA
Lm-LLO-NY-1
GGCTTGTACACAGGGACCTGGCTGCCCGGAATGTGCTAGTCAAGAGTCCC
AACCACGTCA
Lm-LLO-NY-2
GGCTTGTACACAGGGACCTGGCTGCCCGGAATGTGCTAGTCAAGAGTCCC
AACCACGTCA
Lm-LLO-138-1
GGCTTGTACACAGGGACCTGGCTGCCCGGAATGTGCTAGTCAAGAGTCCC
AACCACGTCA
Lm-LLO-138-2
GGCTTGTACACAGGGACCTGGCTGCCCGGAATGTGCTAGTCAAGAGTCCC
AACCACGTCA
Lm-LLO-138-3
GGCTTGTACACAGGGACCTGGCTGCCCGGAATGTGCTAGTCAAGAGTCCC
AACCACGTCA
Lm-LLO-138-4
GGCTTGTACACAGGGACCTGGCTGCCCGGAATGTGCTAGTCAAGAGTCCC
AACCACGTCA
Lm-ddA-164-1
GGCTTGTACACAGGGACCTGGCTGCCCGGAATGTGCTAGTCAAGAGTCCC
AACCACGTCA
Lm-ddA-164-2
GGCTTGTACACAGGGACCTGGCTGCCCGGAATGTGCTAGTCAAGAGTCCC
AACCACGTCA
Lm-ddA-164-4
GGCTTGTACACAGGGACCTGGCTGCCCGGAATGTGCTAGTCAAGAGTCCC
AACCACGTCA
Lm-ddA-164-3
GGCTTGTACACAGGGACCTGGCTGCCCGGAATGTGCTAGTCAAGAGTCCC
AACCACGTCA
Lm-ddA-164-5
GGCTTGTACACAGGGACCTGGCTGCCCGGAATGTGCTAGTCAAGAGTCCC
AACCACGTCA
Lm-ddA164-6
GGCTTGTACACAGGGACCTGGCTGCCCGGAATGTGCTAGTCAAGAGTCCC
AACCACGTCA
Reference

(SEQ ID NO: 33)

AGATTACAGATTTCGGGCTGGCTCGGCTGCTGGACATTGATGAGACAGAG
TACCATGCAG
Lm-LLO-NY-1
AGATTACAGATTTCGGGCTGGCTCGGCTGCTGGACATTGATGAGACAGAG
TACCATGCAG
Lm-LLO-NY-2
AGATTACAGATTTCGGGCTGGCTCGGCTGCTGGACATTGATGAGACAGAG
TACCATGCAG
Lm-LLO-138-1
AGATTACAGATTTCGGGCTGGCTCGGCTGCTGGACATTGATGAGACAGAG
TACCATGCAG
Lm-LLO-138-2
AGATTACAGATTTCGGGCTGGCTCGGCTGCTGGACATTGATGAGACAGAG
TACCATGCAG
Lm-LLO-138-3
AGATTACAGATTTCGGGCTGGCTCGGCTGCTGGACATTGATGAGACAGAG
TACCATGCAG
Lm-LLO-138-4
AGATTACAGATTTCGGGCTGGCTCGGCTGCTGGACATTGATGAGACAGAG
TACCATGCAG
Lm-ddA-164-1
AGATTACAGATTTCGGGCTGGCTCGGCTGCTGGACATTGATGAGACAGAG
TACCATGCAG
Lm-ddA-164-2
AGATTACAGATTTCGGGCTGGCTCGGCTGCTGGACATTGATGAGACAGAG
TACCATGCAG
Lm-ddA-164-3
AGATTACAGATTTCGGGCTGGCTCGGCTGCTGGACATTGATGAGACAGAG
TACCATGCAG
Lm-ddA-164-4
AGATTACAGATTTCGGGCTGGCTCGGCTGCTGGACATTGATGAGACAGAG
TACCATGCAG
Lm-ddA-164-5
AGATTACAGATTTCGGGCTGGCTCGGCTGCTGGACATTGATGAGACAGAG
TACCATGCAG
Lm-ddA164-6
AGATTACAGATTTCGGGCTGGCTCGGCTGCTGGACATTGATGAGACAGAG
TACCATGCAG
Reference

(SEQ ID NO: 34)

ATGGGGGCAAGGTGCCCATCAAATGGATGGCATTGGAATCTATTCTCAGA
CGCCGGTTCA
Lm-LLO-NY-1
ATGGGGGCAAGGTGCCCATCAAATGGATGGCATTGGAATCTATTCTCAGA
CGCCGGTTCA
Lm-LLO-NY-2
ATGGGGGCAAGGTGCCCATCAAATGGATGGCATTGGAATCTATTCTCAGA
CGCCGGTTCA
Lm-LLO-138-1
ATGGGGGCAAGGTGCCCATCAAATGGATGGCATTGGAATCTATTCTCAGA
CGCCGGTTCA
Lm-LLO-138-2
ATGGGGGCAAGGTGCCCATCAAATGGATGGCATTGGAATCTATTCTCAGA
CGCCGGTTCA
Lm-LLO-138-3
ATGGGGGCAAGGTGCCCATCAAATGGATGGCATTGGAATCTATTCTCAGA
CGCCGGTTCA
Lm-LLO-138-4
ATGGGGGCAAGGTGCCCATCAAATGGATGGCATTGGAATCTATTCTCAGA
CGCCGGTTCA
Lm-ddA-164-1
ATGGGGGCAAGGTGCCCATCAAATGGATGGCATTGGAATCTATTCTCAGA
CGCCGGTTCA
Lm-ddA-164-2
ATGGGGGCAAGGTGCCCATCAAATGGATGGCATTGGAATCTATTCTCAGA
CGCCGGTTCA
Lm-ddA-164-3
ATGGGGGCAAGGTGCCCATCAAATGGATGGCATTGGAATCTATTCTCAGA
CGCCGGTTCA
Lm-ddA-164-4
ATGGGGGCAAGGTGCCCATCAAATGGATGGCATTGGAATCTATTCTCAGA
CGCCGGTTCA
Lm-ddA-164-5
ATGGGGGCAAGGTGCCCATCAAATGGATGGCATTGGAATCTATTCTCAGA
CGCCGGTTCA
Lm-ddA-164-6
ATGGGGGCAAGGTGCCCATCAAATGGATGGCATTGGAATCTATTCTCAGA
CGCCGGTTCA
Reference

(SEQ ID NO: 35)

CCCATCAGAGTGATGTGTGGAGCTATGGAGTGACTGTGTGGGAGCTGATG
ACTTTTGGGG
Lm-LLO-NY-1
CCCATCAGAGTGATGTGTGGAGCTATGGAGTGACTGTGTGGGAGCTGATG
ACTTTTGGGG
Lm-LLO-NY-2
CCCATCAGAGTGATGTGTGGAGCTATGGAGTGACTGTGTGGGAGCTGATG
ACTTTTGGGG
Lm-LLO-138-1
CCCATCAGAGTGATGTGTGGAGCTATGGAGTGACTGTGTGGGAGCTGATG
ACTTTTGGGG
Lm-LLO-138-2
CCCATCAGAGTGATGTGTGGAGCTATGGAGTGACTGTGTGGGAGCTGATG
ACTTTTGGGG
Lm-LLO-138-3
CCCATCAGAGTGATGTGTGGAGCTATGGAGTGACTGTGTGGGAGCTGATG
ACTTTTGGGG
Lm-LLO-138-4
CCCATCAGAGTGATGTGTGGAGCTATGGAGTGACTGTGTGGGAGCTGATG
ACTTTTGGGG
Lm-ddA-164-1
CCCATCAGAGTGATGTGTGGAGCTATGGAGTGACTGTGTGGGAGCTGATG
ACTTTTGGGG
Lm-ddA-164-2
CCCATCAGAGTGATGTGTGGAGCTATGGAGTGACTGTGTGGGAGCTGATG
ACTTTTGGGG
Lm-ddA-164-3
CCCATCAGAGTGATGTGTGGAGCTATGGAGTGACTGTGTGGGAGCTGATG
ACTTTTGGGG
Lm-ddA-164-4
CCCATCAGAGTGATGTGTGGAGCTATGGAGTGACTGTGTGGGAGCTGATG
ACTTTTGGGG
Lm-ddA-164-5
CCCATCAGAGTGATGTGTGGAGCTATGGAGTGACTGTGTGGGAGCTGATG
ACTTTTGGGG
Lm-ddA164-6
CCCATCAGAGTGATGTGTGGAGCTATGGAGTGACTGTGTGGGAGCTGATG
ACTTTTGGGG
Reference

(SEQ ID NO: 36)

CCAAACCTTACGATGGAATCCCAGCCCGGGAGATCCCTGATTTGCTGGAG
AAGGGAGAA
Lm-LLO-NY-1
CCAAACCTTACGATGGAATCCCAGCCCGGGAGATCCCTGATTTGCTGGAG
AAGGGAGAA
Lm-LLO-NY-2
CCAAACCTTACGATGGAATCCCAGCCCGGGAGATCCCTGATTTGCTGGAG
AAGGGAGAA
Lm-LLO-138-1
CCAAACCTTACGATGGAATCCCAGCCCGGGAGATCCCTGATTTGCTGGAG
AAGGGAGAA
Lm-LLO-138-3
CCAAACCTTACGATGGAATCCCAGCCCGGGAGATCCCTGATTTGCTGGAG
AAGGGAGAA
Lm-LLO-138-4
CCAAACCTTACGATGNAATCCCAGCCCGGGAGATCCCTGATTTGCTGGAG
AAGGGAGAA
Lm-ddA164-6
CCAAACCTTACGATGGAATCCCAGCCCGGGAGATCCCTGATTTGCTGGAG
AAGGGAGAA
Lm-ddA-164-2
CCAAACCTTACGATGGAATCCCAGCCCGGGAGATCCCTGATTTGCTGGAG
AAGGGAGAA
Lm-LLO-138-2
CCAAACCTTACGATGGAATCCCAGCCCGGGAGATCCCTGATTTGCTGGAG
AAGGGAGAA
Lm-ddA-164-3
CCAAACCTTACGATGGAATCCCAGCCCGGGAGATCCCTGATTTGCTGGAG
AAGGGAGAA
Lm-ddA-164-5
CCAAACCTTACGATGGAATCCCAGCCCGGGAGATCCCTGATTTGCTGGAG
AAGGGAGAA
Lm-ddA-164-1
CCAAACCTTACGATGGAATCCCAGCCCGGGAGATCCCTGATTTGCTGGAG
AAGGGAGAA
Lm-ddA-164-4
CCAAACCTTACGATGGAATCCCAGCCCGGGAGATCCCTGATTTGCTGGAG
AAGGGAGAA
Reference

(SEQ ID NO: 37)

CGCCTACCTCAGCCTCCAATCTGCACCATTGATGTCTACATGATTATGGT
CAAATGTT
Lm-LLO-NY-1
CGCCTACCTCAGCCTCCAATCTGCACCATTGATGTCTACATGATTATGGT
CAAATGTT
Lm-LLO-NY-2
CGCCTACCTCAGCCTCCAATCTGCACCATTGATGTCTACATGATTATGGT
CAAATGTT
Lm-LLO-138-1
CGCCTACCTCAGCCTCCAATCTGCACCATTGATGTCTACATGATTATGGT
CAAATGTT
Lm-LLO-138-2
CGCCTACCTCAGCCTCCAATCTGCACCATTGATGTCTACATGATTATGGT
CAAATGTT
Lm-LLO-138-3
CGCCTACCTCAGCCTCCAATCTGCACCATTGATGTCTACATGATTATGGT
CAAATGTT
Lm-LLO-138-4
CGCCTACCTCAGCCTCCAATCTGCACCATTGATGTCTACATGATTATGGT
CAAATGTT
Lm-ddA-164-1
CGCCTACCTCAGCCTCCAATCTGCACCATTGATGTCTACATGATTATGGT
CAAATGTT
Lm-ddA-164-2
CGCCTACCTCAGCCTCCAATCTGCACCATTGATGTCTACATGATTATGGT
CAAATGTT
Lm-ddA-164-3
CGCCTACCTCAGCCTCCAATCTGCACCATTGATGTCTACATGATTATGGT
CAAATGTT
Lm-ddA-164-4
CGCCTACCTCAGCCTCCAATCTGCACCATTGATGTCTACATGATTATGGT
CAAATGTT
Lm-ddA-164-5
CGCCTACCTCAGCCTCCAATCTGCACCATTGATGTCTACATGATTATGGT
CAAATGTT
Lm-ddA164-6
CGCCTACCTCAGCCTCCAATCTGCACCATTGATGTCTACATGATTATGGT
CAAATGTT
Reference

(SEQ ID NO: 38)

GGATGATTGACTCTGAATGTCGCCCGAGATTCCGGGAGTTGGTGTCAGAA
TTTT
Lm-LLO-NY-1
GGATGATTGACTCTGAATGTCGCCCGAGATTCCGGGAGTTGGTGTCAGAA
TTTT
Lm-LLO-NY-2
GGATGATTGACTCTGAATGTCGCCCGAGATTCCGGGAGTTGGTGTCAGAA
TTTT
Lm-LLO-138-2
GGATGATTGACTCTGAATGTCCCCCGAGATTCCGGGAGTTGGTGTCAAAA
TTTT
Lm-LLO-138-3
GGATGATTGACTCTGAATGTCGCCCGAGATTCCGGGAGTTGGTGTCAGAA
TTTT
Lm-LLO-138-4
GGATGATTGACTCTGAATGTCGCCCGAGATTCCGGGAGTTGGTGTCAGAA
TTTT
Lm-ddA-164-1
GGATGATTGACTCTGAATGTCGCCCGAGATTCCGGGAGTTGGTGTCAGAA
TTTT
Lm-ddA-164-2
GGATGATTGACTCTGAATGTCGCCCGAGATTCCGGGAGTTGGTGTCAGAA
TTTT
Lm-ddA-164-3
GGATGATTGACTCTGAATGTCGCCCGAGATTCCGGGAGTTGGTGTCAGAA
TTTT
Lm-ddA-164-5
GGATGATTGACTCTGAATGTCGCCCGAGATTCCGGGAGTTGGTGTCAGAA
TTTT
Lm-ddA-164-4
GGATGATTGACTCTGAATGTCGCCCGAGATTCCGGGAGTTGGTGTCAGAA
TTTT
Lm-ddA164-6
GGATGATTGACTCTGAATGTCGCCCGAGATTCCGGGAGTTGGTGTCAGAA
TTTT
Reference

(SEQ ID NO: 39)

CACGTATGGCGAGGGACCCCCAGCGTTTTGTGGTCATCCAGAACGAGGAC
TT
Lm-LLO-NY-1
CACGTATGGCGAGGGACCCCCAGCGTTTTGTGGTCATCCAGAACGAGGAC
TT
Lm-LLO-NY-2
CACGTATGGCGAGGGACCCCCAGCGTTTTGTGGTCATCCAGAACGAGGAC
TT
Lm-LLO-138-2
CACGTATGGCGAGGGACCCCCAGCGTTTTGTGGTCATCCAGAACGAGGAC
TT
Lm-LLO-138-3
CACGTATGGCGAGGGACCCCCAGCGTTTTGTGGTCATCCAGAACGAGGAC
TT
Lm-LLO-138-4
CACGTATGGCGAGGGACCCCCAGCGTTTTGTGGTCATCCAGAACGAGGAC
TT
Lm-ddA-164-1
CACGTATGGCGAGGGACCCCCAGCGTTTTGTGGTCATCCAGAACGAGGAC
TT
Lm-ddA-164-2
CACGTATGGCGAGGGACCCCCAGCGTTTTGTGGTCATCCAGAACGAGGAC
TT
Lm-ddA-164-3
CACGTATGGCGAGGGACCCCCAGCGTTTTGTGGTCATCCAGAACGAGGAC
TT
Lm-ddA-164-5
CACGTATGGCGAGGGACCCCCAGCGTTTTGTGGTCATCCAGAACGAGGAC
TT
Lm-ddA-164-6
CACGTATGGCGAGGGACCCCCAGCGTTTTGTGGTCATCCAGAACGAGGAC
TT

Alignment of EC1 (399-758 by of HER2/Neu)

Reference

(SEQ ID NO: 40)

CCCAGGCAGAACCCCAGAGGGGCTGCGGGAGCTGCAGCTTCGAAGTCTCA
CAGAGATCCT
Lm-LLO-138-1
CCCAGGCAGAACCCCAGAGGGGCTGCGGGAGCTGCAGCTTCGAAGTCTCA
CAGAGATCCT
Lm-LLO-138-2
CCCAGGCAGAACCCCAGAGGGGCTGCGGGAGCTGCAGCTTCGAAGTCTCA
CAGAGATCCT
Lm-ddA-164-1
CCCAGGCAGAACCCCAGAGGGGCTGCGGGAGCTGCAGCTTCGAAGTCTCA
CAGAGATCCT
LmddA-164-2
CCCAGGCAGAACCCCAGAGGGGCTGCGGGAGCTGCAGCTTCGAAGTCTCA
CAGAGATCCT
LmddA-164-3
CCCAGGCAGAACCCCAGAGGGGCTGCGGGAGCTGCAGCTTCGAAGTCTCA
CAGAGATCCT
LmddA164-4
CCCAGGCAGAACCCCAGAGGGGCTGCGGGAGCTGCAGCTTCGAAGTCTCA
CAGAGATCCT
Reference

(SEQ ID NO: 41)

GAAGGGAGGAGTTTTGATCCGTGGGAACCCTCAGCTCTGCTACCAGGACA
TGGTTTTGTG
Lm-LLO-138-1
GAAGGGAGGAGTTTTGATCCGTGGGAACCCTCAGCTCTGCTACCAGGACA
TGGTTTTGTG
Lm-LLO-138-2
GAAGGGAGGAGTTTTGATCCGTGGGAACCCTCAGCTCTGCTACCAGGACA
TGGTTTTGTG
Lm-ddA-164-1
GAAGGGAGGAGTTTTGATCCGTGGGAACCCTCAGCTCTGCTACCAGGACA
TGGTTTTGTG
LmddA-164-2
GAAGGGAGGAGTTTTGATCCGTGGGAACCCTCAGCTCTGCTACCAGGACA
TGGTTTTGTG
LmddA-164-3
GAAGGGAGGAGTTTTGATCCGTGGGAACCCTCAGCTCTGCTACCAGGACA
TGGTTTTGTG
LmddA164-4
GAAGGGAGGAGTTTTGATCCGTGGGAACCCTCAGCTCTGCTACCAGGACA
TGGTTTTGTG
Reference

(SEQ ID NO: 42)

CCGGGCCTGTCCACCTTGTGCCCCCGCCTGCAAAGACAATCACTGTTGGG
GTGAGAGTCC
Lm-LLO-138-1
CCGGGCCTGTCCACCTTGTGCCCCCGCCTGCAAAGACAATCACTGTTGGG
GTGAGAGTCC
Lm-LLO-138-2
CCGGGCCTGTCCACCTTGTGCCCCCGCCTGCAAAGACAATCACTGTTGGG
GTGAGAGTCC
Lm-ddA-164-1
CCGGGCCTGTCCACCTTGTGCCCCCGCCTGCAAAGACAATCACTGTTGGG
GTGAGAGTCC
LmddA-164-2
CCGGGCCTGTCCACCTTGTGCCCCCGCCTGCAAAGACAATCACTGTTGGG
GTGAGAGTCC
LmddA-164-3
CCGGGCCTGTCCACCTTGTGCCCCCGCCTGCAAAGACAATCACTGTTGGG
GTGAGAGTCC
LmddA164-4
CCGGGCCTGTCCACCTTGTGCCCCCGCCTGCAAAGACAATCACTGTTGGG
GTGAGAGTCC
Reference

(SEQ ID NO: 43)

GGAAGACTGTCAGATCTTGACTGGCACCATCTGTACCAGTGGTTGTGCCC
GGTGCAAGGG
Lm-LLO-138-1
GGAAGACTGTCAGATCTTGACTGGCACCATCTGTACCAGTGGTTGTGCCC
GGTGCAAGGG
Lm-LLO-138-2
GGAAGACTGTCAGATCTTGACTGGCACCATCTGTACCAGTGGTTGTGCCC
GGTGCAAGGG
Lm-ddA-164-1
GGAAGACTGTCAGATCTTGACTGGCACCATCTGTACCAGTGGTTGTGCCC
GGTGCAAGGG
LmddA-164-2
GGAAGACTGTCAGATCTTGACTGGCACCATCTGTACCAGTGGTTGTGCCC
GGTGCAAGGG
LmddA-164-3
GGAAGACTGTCAGATCTTGACTGGCACCATCTGTACCAGTGGTTGTGCCC
GGTGCAAGGG
LmddA164-4
GGAAGACTGTCAGATCTTGACTGGCACCATCTGTACCAGTGGTTGTGCCC
GGTGCAAGGG
Reference

(SEQ ID NO: 44)

CCGGCTGCCCACTGACTGCTGCCATGAGCAGTGTGCCGCAGGCTGCACGG
GCCCCAAGCA
Lm-LLO-138-1
CCGGCTGCCCACTGACTGCTGCCATGAGCAGTGTGCCGCAGGCTGCACGG
GCCCCAAGCA
Lm-LLO-138-2
CCGGCTGCCCACTGACTGCTGCCATGAGCAGTGTGCCGCAGGCTGCACGG
GCCCCAAGCA
Lm-ddA-164-1
CCGGCTGCCCACTGACTGCTGCCATGAGCAGTGTGCCGCAGGCTGCACGG
GCCCCAAGCA
LmddA-164-2
CCGGCTGCCCACTGACTGCTGCCATGAGCAGTGTGCCGCAGGCTGCACGG
GCCCCAAGTA
LmddA-164-3
CCGGCTGCCCACTGACTGCTGCCATGAGCAGTGTGCCGCAGGCTGCACGG
GCCCCAAGTA
LmddA164-4
CCGGCTGCCCACTGACTGCTGCCATGAGCAGTGTGCCGCAGGCTGCACGG
GCCCCAAGTA

### Example 7

### Peripheral Immunization with ADXS31-164 can Delay the Growth of a Metastatic Breast Cancer Cell Line in the Brain

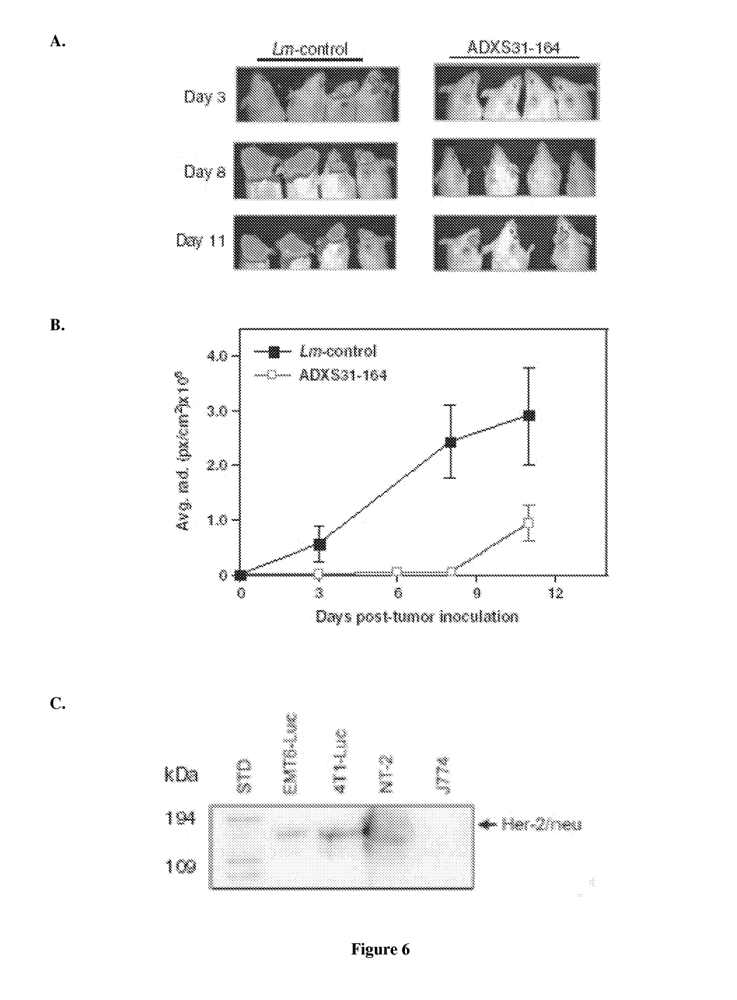

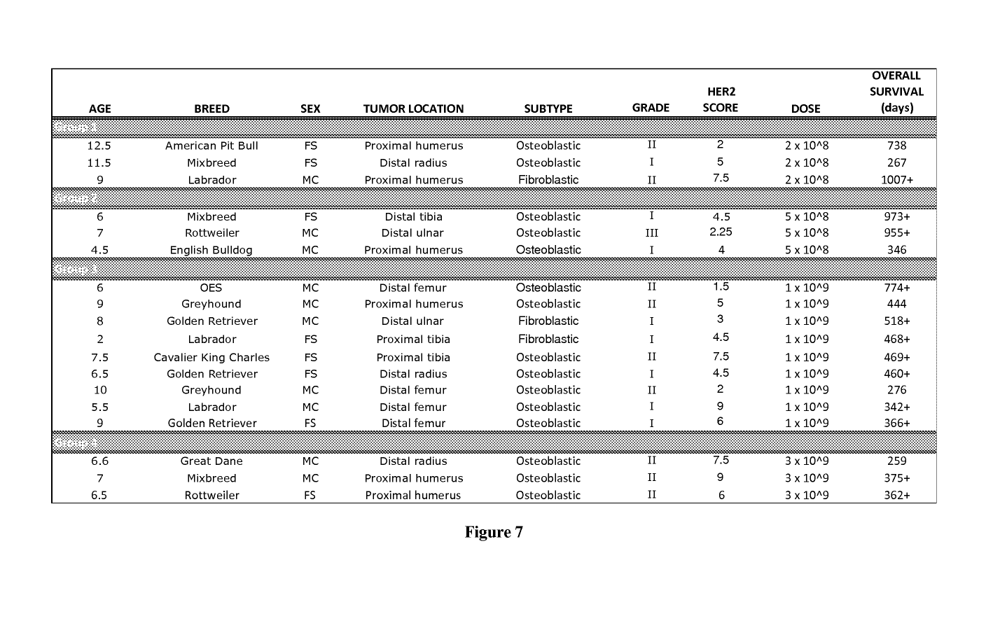

Mice were immunized IP with ADXS31-164 or irrelevant Lm-control vaccines and then implanted intra-cranially with 5,000 EMT6-Luc tumor cells, expressing luciferase and low levels of HER2/neu (FIG. 6C). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIG. 6A and B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

### Example 8

### Treatment of Canine Osteosarcoma by Immunization with ADXS31-164

Canine Osteosarcoma is a cancer of long (leg) bones that is a leading killer of large dogs over the age of 10 years. Standard treatment is amputation immediately after diagnosis, followed by chemotherapy. Invariably, however, the cancer metastasizes to the lungs. With chemotherapy, dogs survive about 18 months compared to 6-12 months, without treatment. The HER2 antigen is believed to be present in up to 50% of osteosarcoma. ADXS31-164 creates an immune attack on cells expressing this antigen and has been developed to treat human breast cancer.

Dogs with a histological diagnosis of osteosarcoma and evidence of expression of HER2/neu by malignant cells are eligible for enrollment.

Canine Osteosarcoma Trial

In the first regiment the limbs are amputated, followed by round of chemotherapy treatment. 3 doses of Her-2 vaccine are subsequently administered with or without a 6 month interval booster.

All dogs are to receive 4 weeks of carboplatin therapy. Four weeks after the last carboplatin dose, dogs are to receive ADXS-HER2 once every three weeks for a total of 3 doses. Group 1 (3 dogs) receive 1×10⁸ CFU per dose, Group 2 (3 dogs) each receive 5×10⁸ CFU per dose and Group 3 (3 dogs) receives 1×10⁹ CFU per dose. Additional dogs are added to a Group to gather more data should if a potentially dose limiting toxicities, be observed. Therefore 9-18 dogs may be treated in the initial study.

In the second regiment, the same as the first regiment is repeated with the exception that only a single dose of vaccine is administered before chemotherapy (1 month before)for a total of 4 doses.

Further, in both regiments a single dose is administered a month after chemotherapy.

### Example 9

### Phase 1 Dose Escalation Study Evaluating the Safety of ADXS-cHER2 in Companion Dogs with HER2/NEU Overexpressing Canine Osteosarcoma

A pilot phase I dose escalation study was performed to determine the dose of a L. monocytogenes expressing human HER2/neu recombinant vaccine that can safely and effectively stimulate tumor-specific immunity in dogs with osteosarcoma. The tumors of all dogs presenting to PennVet for limb amputation due to suspected or confirmed OSA were routinely harvested and evaluated histopathologically to confirm the diagnosis of OSA. In addition, tumor sections from all dogs were evaluated by IHC and Western blot analysis to determine whether the tumor expresses HER2/neu. Only dogs with a histological diagnosis of OSA and evidence of expression of HER2/neu by malignant cells were eligible for enrollment. Single cell suspensions of tumor tissue taken at surgery are cryopreserved and used as autologous tumor targets in chromium release assays to determine anti-tumor immunity.

Up to 18 privately owned dogs with appendicular OSA and confirmed expression of Her2-neu were enrolled (FIG. 7). At enrollment (3 weeks post last carboplatin treatment), all dogs received basic clinical laboratory tests including a Complete Blood Count (CBC), Chemistry Screen (CS) and urinalysis (UA) and a baseline evaluation of cardiac function by echocardiography and measurement of cardiac-specific Troponin I (cTnI) levels. Thoracic radiographs are taken to determine whether pulmonary metastases are present. Only dogs with no evidence of pulmonary metastases were eligible for inclusion in the study. At the time of enrollment, peripheral blood mononuclear cells (PBMCs) are collected to assess baseline levels of anti-tumor immunity (see Assessment of anti-tumor immunity). Furthermore, blood was taken to evaluate baseline immune function to ensure they are no longer immune suppressed by carboplatin. Only dogs with functionally intact immune systems were eligible to receive the Listeria vaccine.

Lm Recombinant Dosing and Data Capture

All dogs were vaccinated using a single ADXS31-164ADXS31-164 recombinant vaccine. The first Lm-huHer2-neu vaccine were given three weeks after the last carboplatin dose and were given once every three weeks after this for a total of 3 doses (FIG. 7).

Group 1 (3 dogs) received the ADXS31-164 (Lm-human chimericHER2/neu) vaccine at 1×10⁸ CFU per dose, Group 2 (3 dogs) each received 5×10⁸ CFU per dose, Group 3 (3 dogs) receive 1×10⁹ CFU per dose, and 3.3×10⁹ CFU per dose (1 dog). Recombinant Lm are administered as a slow intravenous infusion over 30 minutes. The dose chosen for Group 1 is the established safe dose for the chimeric ADXS31-164 recombinant in mice. In humans, the non-toxic dose for Lovaxin C is only one log higher than that established in mice, and this dose is the dose evaluated in Group 3 in this pilot trial.

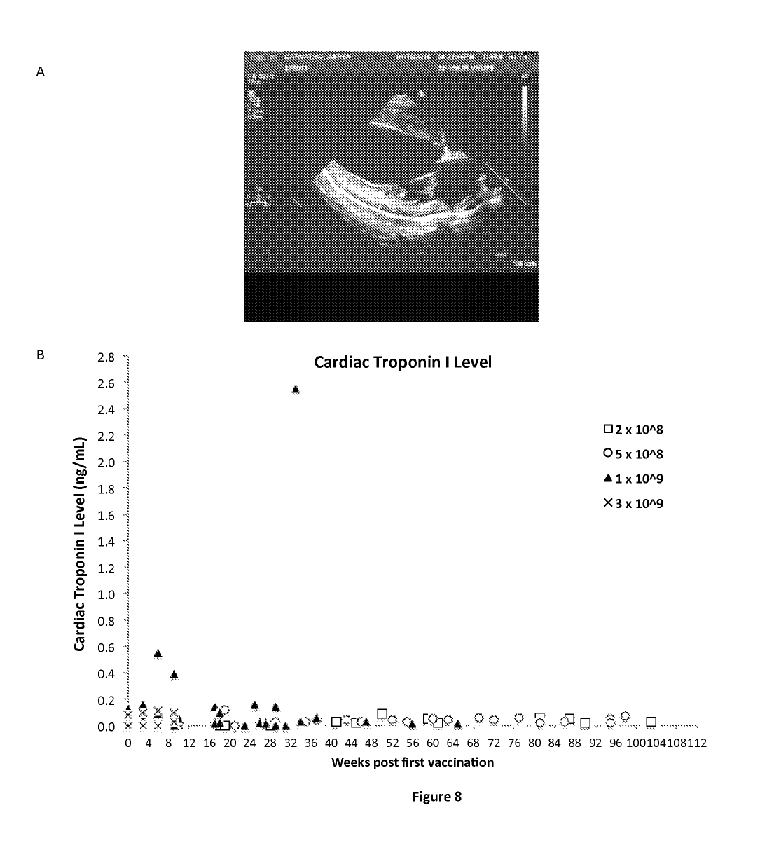

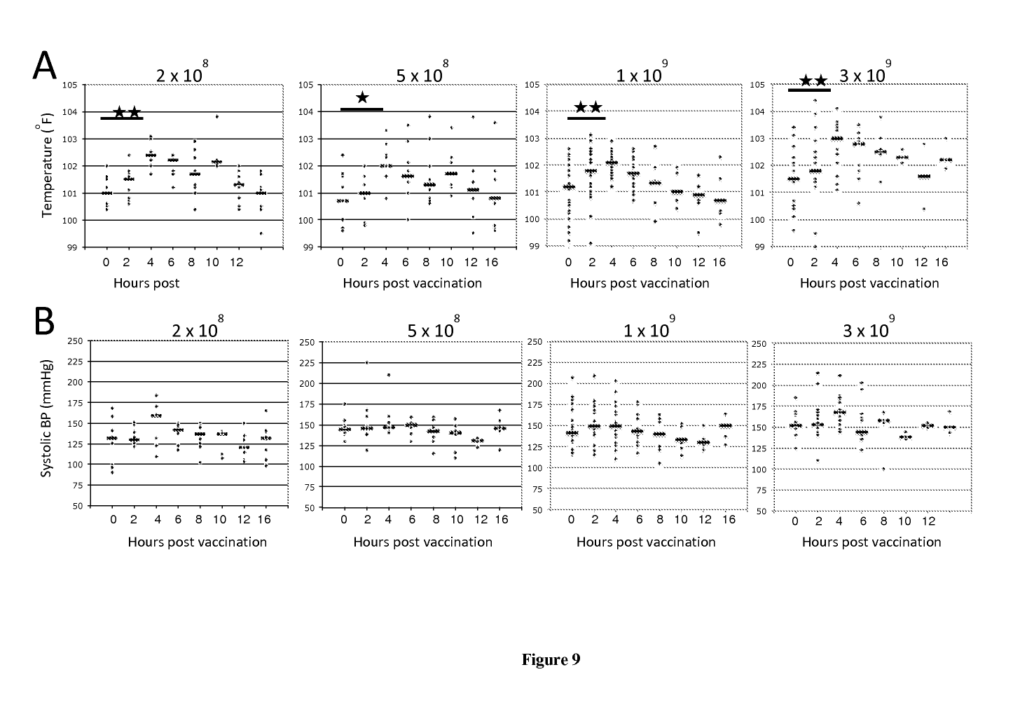

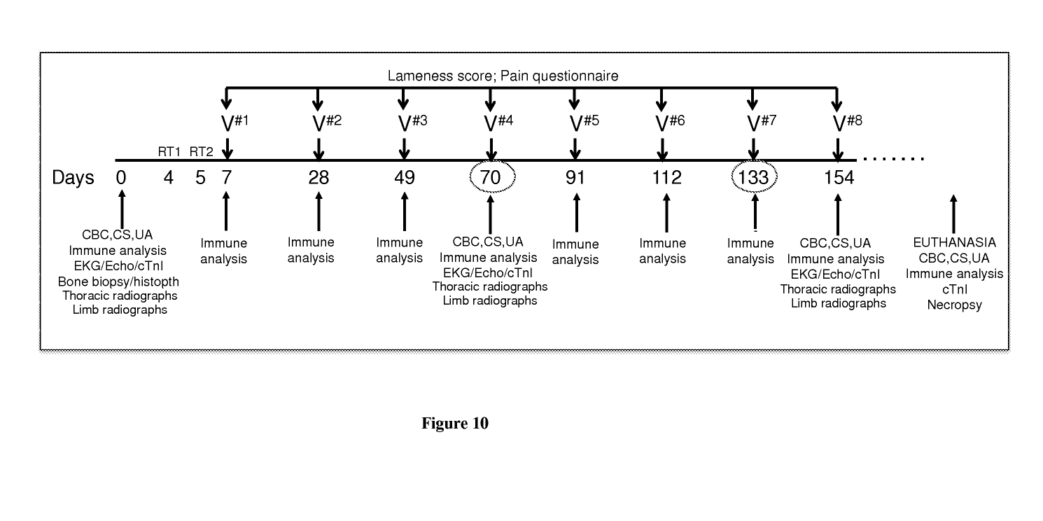

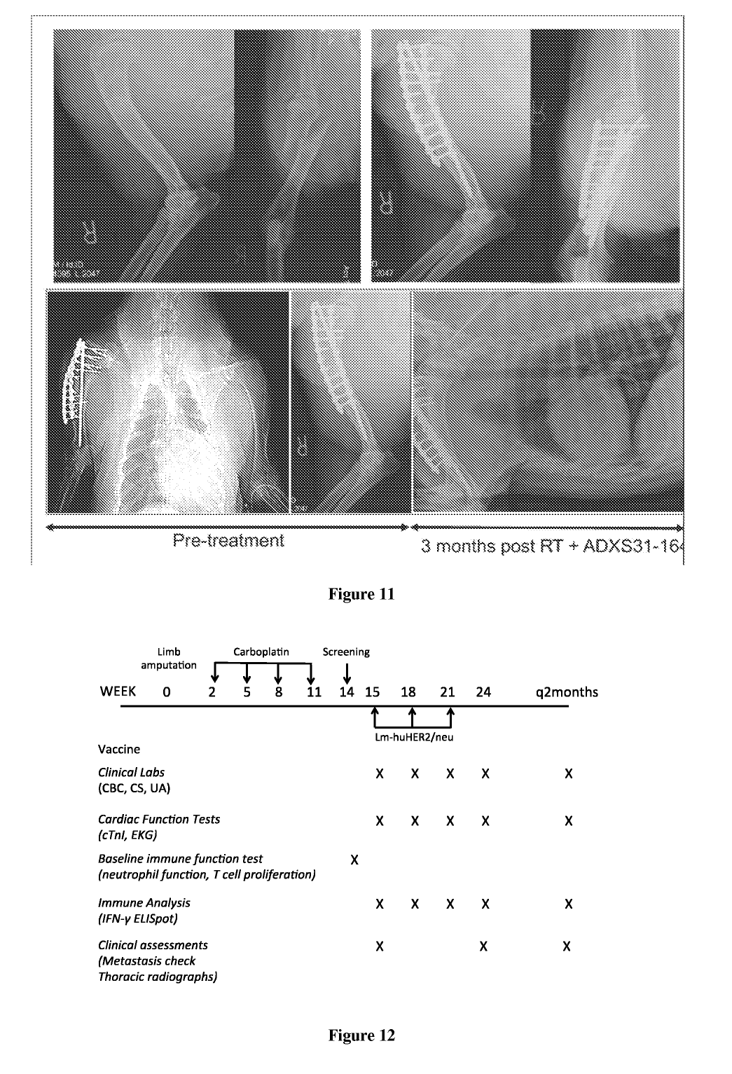

At the time of Lm administration, dogs were monitored for evidence of systemic adverse effects. During infusion, heart rate and rhythm was monitored by ECG and respiratory rate are recorded. Further, heart damage was monitored using ultrasound and by measuring Troponin I levels (FIG. 8). Following infusion, dogs are monitored closely for 48 hours. Core body temperature is monitored continuously for <12 hours post infusion using the Vital Sense continuous body temperature monitoring system by MiniMitter Respironics (routinely used in our Veterinary Clinical Trials Center, VCIC). Pulse rate, rhythm and quality, respiratory rate and effort, were monitored and recorded every hour for the first 6 hours then every 4 hours thereafter, as well as blood pressure and temperature (FIG. 9). All symptoms consistent with immune stimulation are noted and fluids, analgesics, anti-emetics and anti-histamines are used as necessary to control severe reactions. All dogs were observed six times a day and any signs of toxicological effects of the recombinants including discomfort, lethargy, nausea, vomiting and diarrhea were recorded. Blood samples were taken at 24, 48 and 72 hours after the first ADXS31-164 vaccine for cultures to assess the clearance of Lm after systemic administration.

Assessment of Anti-Tumor Immunity

Three weeks following the last carboplatin dose, dogs receive a routine clinical examination and baseline blood work including CBC, CS, UA and cTnI levels. PBMCs are taken at this time for baseline evaluation of anti-tumor immunity. Repeat immune assessment is performed at the time of each vaccination and three weeks after the last vaccination. PBMCs are analyzed for HER2/neu specific T cell responses by CFSE proliferation, cytokine production (ELISpot and qRT-PCR) and CTL assay against autologous tumor targets as outlined below (FIG. 12).

Results

To date we have performed a total of 41 infusions of ADXS31-164 in 16 dogs.

| Number of | Number of | |
|---|---|---|
| dogs | infusions | Rationale |
| 1 | 5 | Two additional infusions post priming series to |
| | | treat metastatic disease |
| 4 | 4 | One additional infusion post priming series to |
| | | maintain tumor free status |
| 4 | 3 | Finished scheduled priming series |
| 1 | 2 | Succumbed to metastatic disease prior to finish |
| | | of priming course |
| 2 | 1 | Succumbed to metastatic disease prior to finish |
| | | of priming course |
| 4 | 1 | Priming course of vaccinations underway |

ADXS31-164 dose has ranged from 1×10⁸, 5×10⁸, 1×10⁹ and 3.3×10⁹ CFU.

| | Total number | | |
|---|---|---|---|
| Dose | of doses | Number | |
| received | administered | of dogs | Reported side effects |
| 1 × 10⁸ | 9 | 3 | Fever, nausea, vomiting, |
| | | | elevated liver enzymes |
| 5 × 10⁸ | 9 | 3 | Fever, nausea, vomiting, |
| | | | elevated liver enzymes |
| 1 × 10⁹ | 17 | 10 | Fever, nausea, vomiting, |
| | | | elevated liver enzymes, |
| | | | thrombocytopenia |
| 3.3 × 10⁹ | 1 | 1 | Nausea, vomiting, |

Standard Operating Procedure for Vaccine Administration

A standard operating procedure was developed for the administration of ADXS31-164. One hour prior to vaccination patients receive 2 mg/kg diphenhydramine via intramuscular injection and 0.2 mg/kg ondansetron as a slow intravenous push. The vaccine was kept at −80° C. and thawed patient-side. It was administered in 200 mls of 0.9% NaCl over 30 mins The infusion line is then flushed with 30 mls of Plasmalyte. Dogs are sent home with a three day course of amoxicillin (to start 72 hours post vaccination) and a 7 day course of liver supplement (S-adenosyl-methionine) that aids in cellular growth and repair.

The primary endpoint of the study was to determine the maximum tolerated dose of ADXS31-164.

Doses up to 3.3×10⁹ were well tolerated in dogs ranging in body weight from 25 kg to 67 kg. All side effects reported were grade I toxicities and the maximum tolerated dose has yet to be reached. Side effects routinely occurred within 2-4 hours of vaccine administration. High fevers usually resolved with intravenous isotonic fluids delivered at maintenance rate (4 mls/kg/hour) for 2-4 hours. In two cases where fevers reached 104.7 and above, a single subcutaneous injection of carprofen induced normothermia within 1-2 hours. Nausea and vomiting was usually self-limiting but in cases where several episodes are noted, 1 mg/kg cerenia is administered and this was very effective at preventing further nausea and vomiting. A total of 5 dogs developed mild, grade I elevations in liver enzymes within 48 hours of vaccine administration—these resolved by one week post vaccination.

Clearance of Listeria

After performing blood cultures on all 16 dogs vaccinated to date there was no detectable listeria in the peripheral circulation of any of the dogs at 24 hours post vaccination. Shedding of listeria in the urine and feces of vaccinated dogs was not assessed.

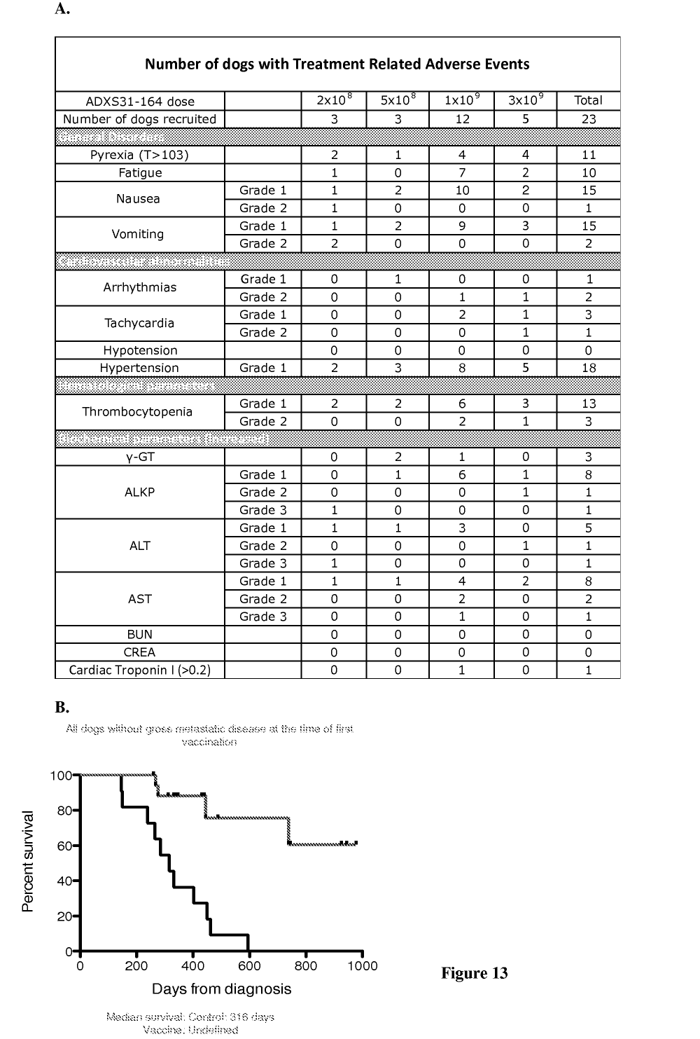

Secondary endpoints for the study are progression-free survival and overall survival. A statistically significant overall survival advantage in dogs with osteosarcoma has been observed when ADXS31-164 is administered after limb amputation and 4 doses of carboplatin. Early results from the first two dose groups (6 dogs) show a significant survival advantage in dogs that received ADXS31-164 compared to 6 dogs whose owners elected not to participate in the trial but who were followed for survival (p=0.003) (FIG. 13). The mean survival time for unvaccinated dogs is 239.5 days. The mean survival time for vaccinated dogs has not yet been reached. This remains true when all dogs within the intent to treat group are included in analysis.

In conclusion, there was no evidence of significant short or long-term side effects on the cardiovascular, hematopoietic, hepatic, or renal systems. Moreover, administration of ADXS31-164 in the presence of minimal residual disease can delay/prevent metastatic disease and prolong overall survival of dogs with HER2/neu positive osteosarcoma.

### Example 10

### Phase 1 Clinical Trial Evaluating ADXS31-164 in Spontaneous Canine Model of Osterosarcoma (OSA)

Materials and Methods

Vaccine Manufacture

Design and generation of ADXS31-164. Briefly, the dal dat actA mutant strain of Listeria monocytogenes (Lm) was transfected with the pADV plasmid carrying a chimeric human HER2/neu construct. The construct contains 2 extracellular domains (EC1 and EC2) and one intracellular domain (IC1) of the human HER2/neu molecule that contain the majority of HLA-A2 restricted immunodominant epitopes, fused to a truncated listeriolysin O construct. The transfer plasmid also contains the bacillus p60 dal gene and is maintained within the mutant Lm via auxotrophic complementation. There is no bacterial resistance cassette. Vaccines were manufactured by Vibalogics GmbH (Cuxhaven, Germany) and stored at −80° C. prior to use.

Histopathology, Staging and Immunohistochemistry

Histopathological assessment of all primary appendicular osteosarcoma tumors was performed by a board certified veterinary pathologist (J.E.). Tumors were described as osteoblastic, chondroblastic, fibroblastic and telangiectatic based on histological features. Primary tumors were scored based on mitotic index, nuclear pleomorphism and the amount of matrix and necrosis present. Histological scores were converted into a grade (I, II or III).

For HER2/neu staining, 5 micron thick serial sections of formalin fixed, decalcified, paraffin embedded tissues were mounted on negatively charged glass slides. Sections were heated at 80° C. for 20 minutes, immersed in Pro Par (clearant) and rehydrated in ethanol. Antigen retrieval was performed by boiling sections in sodium citrate buffer (pH ˜9.0). Endogenous peroxidase was blocked using 3% hydrogen peroxide. Staining was performed with a rabbit anti-human HER2/neu antibody (Neu(c-18):sc-284, Santa Cruz Biotecnology) or a rabbit IgG isotype (Universal Negative Control serum, NC498, Biocare Medical). Bound antibody was detected using the Universal Streptavadin-Biotin2 System (DAKO/LSAB2, HRP). Tissues were stained with 3,3′-diaminobenzidine solution (DAKO) and counterstained with hematoxylin. Slides were viewed using a Nikon E600 infinity corrected upright microscope. Bright field images were acquired using a Nikon Digital Sight DS-Fi1 color camera and a NIS-Element BR3.0 for image analysis. Tissue sections were evaluated and scored for HER2/neu positivity by a board certified pathologist (J.E.) based on the percentage of neoplastic cells staining for HER2/neu (<10%=1, 10%-50%=2, >50%=3) and the intensity of HER2/neu staining (weak=1, moderate=2, strong=3). Scores were based on cells analyzed within 10 hpf for each tissue section. A combined HER2/neu score was obtained by multiplying the two separate scores given for percentage of tumor cells positive for HER2/neu staining and HER2/neu staining intensity. Only dogs with greater than 10% of their tumor cells staining positive for HER2/neu were eligible for trial enrollment.

Eligibility Criteria and Clinical Trial Design

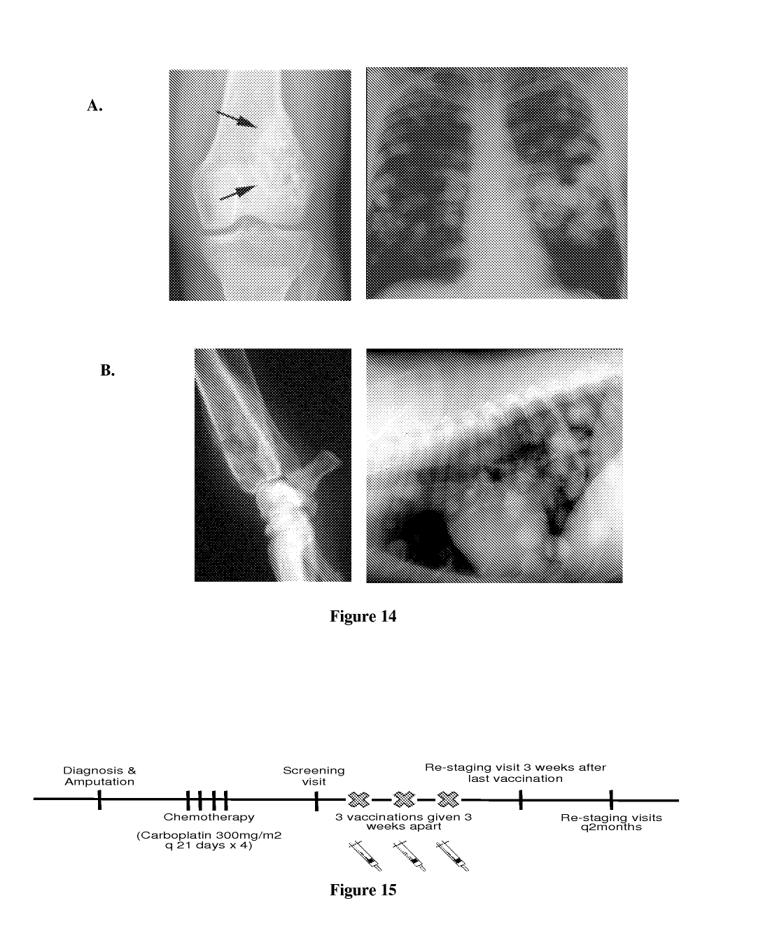

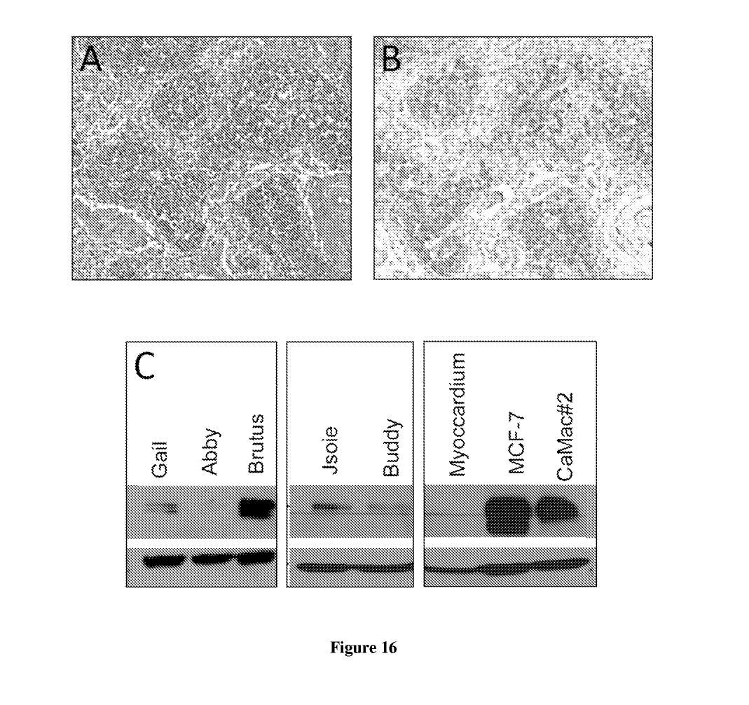

Dogs with a histopathological and immunohistochemical diagnosis of HER2/neu positive OSA that had undergone primary tumor removal either by limb amputation or limb-sparing surgery and had received 4 doses of 300 mg/m² carboplatin given once every 3 weeks (or once every 4 weeks if myelosuppression occurred) as adjuvant chemotherapy were eligible for screening. Dogs were screened three weeks after their last carboplatin treatment. A thorough physical examination, Complete Blood Count (CBC), Chemistry Screen (CS) and Urinalysis (UA) were performed to determine general health status. Basic innate and adaptive immune function was tested using a flow cytometric neutrophil oxidative burst assay and mitogen-induced lymphocyte proliferation assay respectively. Baseline cardiac status was evaluated by electrocardiography, echocardiography and serum cardiac troponin I levels. Thoracic radiographs were performed to determine the presence of pulmonary metastatic disease (see FIG. 14B). Only those dogs found to be systemically healthy with intact innate and adaptive immune function, no evidence of underlying cardiac disease and no evidence of pulmonary metastatic disease were eligible for enrollment. Dogs that died during the course of the study underwent necropsy. The presence and location of metastatic disease was recorded and histopathology and immunohistochemistry to evaluate HER2/neu expression in metastatic lesions were performed.

Immune Analysis

Neutrophil oxidative burst assay. Red blood cells in sodium heparin anti-coagulated blood were lysed using 0.83% NH₄Cl and the remaining white blood cells were washed twice in 1× PBS. Cells were labeled with 15 ug/ml of dihydrorhodamine 123 (DHR-123; Molecular Probes, Grand Island, N.Y.) and activated with 3 nM phorbol 12-myristate 13-acetate (PMA, Sigma, St. Louis, Mo.) for 30 minutes at 37° C. Cells were placed on ice for 15 minutes prior to flow cytometric analysis. Cells were acquired on a FACS Canto cytometer (BD Biosciences, San Jose, CA) and analyzed using FloJo software (Treestar, San Carlos, Calif.).

Lymphocyte proliferation assay. Peripheral Blood Mononuclear Cells (PBMCs) were isolated from sodium heparin anti-coagulated whole blood by density centrifugation. PBMCs were washed twice in 1× PBS and counted. Cells were labeled with 5 uM CFSE and stimulated with 1.25 uM Concanavalin A at 37° C. for 5 days. Cells were harvested, washed twice in FACS buffer, labeled with APC-conjugated rat anti-canine CD4 and PE conjugated rat anti-canine CD8 antibodies (Serotec, Raleigh, N.C.) and analyzed by flow cytometry. For immune function analysis, peripheral blood taken from healthy colony dogs (IACUC #804197) was used as a positive control.

T cell subset analysis. PBMCs taken at baseline, prior to each vaccination, at re-stage and at every 2 months thereafter were analyzed for CD4 and CD8 T cell subsets. Briefly, cryopreserved cells were thawed and washed twice in FACS buffer (1× PBS, 0.2% BSA fraction V, and 4 mM sodium azide) prior to surface staining with mouse anti-canine CD3, PE-labeled rat anti-dog CD8 or Alexa-labeled rat anti-dog CD4 (Serotec, Raleigh, NC). Cells were incubated with the vital dye 7-ADD immediately prior to flow cytometric acquisition. Total CD4⁺ and CD8⁺ T cell numbers were calculated from the flow cytometric percentages and total lymphocyte counts determined using a Cell Dyn 3700CS Hematology analyzer.

Vaccine Administration

Prior to vaccination, dogs received the 5HT3 antagonist ondansetron (0.2 mg/kg) intravenously and the H1 receptor blocker, diphenhydramine (2 mg/kg) intramuscularly to prevent nausea and anaphylaxis respectively. A standard 3+3 clinical trial design was employed. ADXS31-164 was administered at the following doses; Group 1 (2×10⁸ CFU) , Group 2 (5×10⁸ CFU), Group 3 (1×10⁹ CFU) and Group 4 (3.3×10⁹ CFU). ADXS31-164 was diluted in 100 mls 0.9% NaCl (Groups 1 and 2) and 200 mls 0.9% NaCl (Groups 3 and 4) and administered intravenously over 30 minutes. Temperature, pulse, respiratory rate, heart rate and rhythm (by EKG) and blood pressure were monitored every hour following infusion. In cases where body temperature exceeded 103° F., dogs were placed on intravenous Plasmalyte at 4 mls/kg/hr until their temperature fell below 103° F. Dogs were monitored every hour for signs of lethargy, nausea or vomiting. Blood samples were drawn 24 hours and one week post vaccination to assess for any changes in hematological or biochemical parameters and blood cultures were performed at 24 hours post vaccination to determine persistance of live bacteria in the blood stream. All dogs received a short course of amoxycillin and S-Adenosylmethionine (SAMe) 72 hours after vaccination to kill any remaining listeria and provide anti-oxidant support to the liver.

Owners with dogs that were free of metastatic disease at least 5 months after receiving the last vaccine in the initial series were offered the option to receive a booster vaccine at a standard dose of 1×10⁹ CFU. Booster vaccines were administered as described and dogs were monitored after infusion as described above.

Toxicity

Toxicity was graded according to the Veterinary Co-operative Oncology Group—Common Terminology Criteria for Adverse Events (VCOG-CTCAE). Assessment of cardiac toxicity was performed through serial electrocardiograms, echocardiograms and serum cardiac troponin I levels at baseline, at the time of each vaccination, 3 weeks after the last vaccination and every 2 months thereafter until death. Parameters assessed included Left Ventricular Fractional Shortening (LVFS) and Left Ventricular Internal Dimension in diastole (LVIDd) and Left Ventricular Internal Dimension in systole (LVIDs). LVIDd and LVIDs were normalized to body weight to account for the wide range of body size amongst dogs.

ELISpot Analysis

Cryopreserved PBMC from each indicated time point were thawed, rested overnight at 37° C. and then counted. Cells were stimulated with 2.5 uM pools of overlapping human HER2/Neu peptides (1 lmers overlapping by 5 amino acids) that represent the EC1, EC2 and IC1 domains of HER2/Neu present in the chimeric vaccine, and recombinant human IL-2 (Invitrogen, Fredrick, Md.) for 5 days. Cells were harvested, washed twice in 1 x PBS and counted. IFN-γ ELISpot assays were performed according to the manufacturer's protocol using a commercial canine IFN-γ ELISpot assay kit (R&D Systems, Minneapolis, Minn.). Briefly, 0.8-2×10⁵ stimulated cells were incubated with 2.5 uM of EC1, EC2 or IC1 peptide pools plus IL-2 or IL-2 alone (to determine background counts). All assays were performed in duplicates. Plates were developed according to the manufacturer's instructions. Spots were counted using a CTL-Immunospot analyzer (C.T.L, Shaker Heights, OH)

Primary and Secondary Outcome Measures

Time To Metastasis (TTM) was calculated as the time between amputation and development of metastatic disease. OSA Specific Survival was calculated as the time between amputation and death. Patients that died of unrelated causes were censored at the time of their death.

### Results

Eighteen dogs that fulfilled the eligibility criteria were enrolled in this phase I clinical trial. The age, breed, sex, tumor location, subtype, grade and HER2/neu status were recorded (Table 4). A standard 3+3 clinical trial design was employed. ADXS31-164 was administered at the following doses; Group 1: 2×10⁸ CFU (n=3), Group 2: 5×10⁸ CFU (n=3), Group 3: 1×10⁹ CFU (n=9), and Group 4: 3×10⁹ CFU (n=3). Five additional dogs with pre-existing pulmonary metastatic disease, identified at the time of screening also received ADXS31-164 on a compassionate care basis (Table 4). Four of these dogs had strong HER2/neu staining in >50% of neoplastic cells from their primary tumor. Three of these dogs had multiple pulmonary metastatic nodules and two dogs had a single metastatic nodule at screening. Dogs with multiple pulmonary nodules received one vaccine each before disease progression and withdrawal from the study for alternative treatments. The two dogs with single nodules received the full course of three vaccines each. Dogs with pre-exisiting metastatic disease received either 1×10⁹ CFU (n=3) or 3×10⁹ CFU (n=2) ADXS31-164 (Table 5).

FIG. 15 shows a schematic of the time-line of the phase 1 clinical trial, wherein three vaccinations were administered following amputation and follow-up chemotherapy.

TABLE 4
Signalment and tumor characteristics of enrolled dogs

| | | | | | | | | |
|---|---|---|---|---|---|---|---|---|
| | | | | | | | | OVERALL |
| | | | | | | HER2 | | SURVIVAL |
| AGE | BREED | SEX | TUMOR LOCATION | SUBTYPE | GRADE | SCORE | DOSE | (days) |

Group 1

| | | | | | | | | |
|---|---|---|---|---|---|---|---|---|
| 12.5 | American Pit Bull | FS | Proximal humerus | Osteoblastic | II | 2 | 2 × 10\hat{ }8 | 738 |
| 11.5 | Mixbreed | FS | Distal radius | Osteoblastic | I | 5 | 2 × 10\hat{ }8 | 267 |
| 9 | Labrador | MC | Proximal humerus | Fibroblastic | II | 7.5 | 2 × 10\hat{ }8 | 977+ |

Group 2

| | | | | | | | | |
|---|---|---|---|---|---|---|---|---|
| 6 | Mixbreed | FS | Distal tibia | Osteoblastic | I | 4.5 | 5 × 10\hat{ }8 | 943+ |
| 7 | Rottweiler | MC | Distal ulnar | Osteoblastic | III | 2.25 | 5 × 10\hat{ }8 | 925+ |
| 4.5 | English Bulldog | MC | Proximal humerus | Osteoblastic | I | 4 | 5 × 10\hat{ }8 | 346 |

Group 3

| | | | | | | | | |
|---|---|---|---|---|---|---|---|---|
| 6 | OES | MC | Distal femur | Osteoblastic | II | 1.5 | 1 × 10\hat{ }9 | 744+ |
| 9 | Greyhound | MC | Proximal humerus | Osteoblastic | II | 5 | 1 × 10\hat{ }9 | 444 |
| 8 | Golden Retriever | MC | Distal ulnar | Fibroblastic | I | 3 | 1 × 10\hat{ }9 | 488+ |
| 2 | Labrador | FS | Proximal tibia | Fibroblastic | I | 4.5 | 1 × 10\hat{ }9 | 438+ |
| 7.5 | Cavalier King Charles | FS | Proximal tibia | Osteoblastic | II | 7.5 | 1 × 10\hat{ }9 | 439+ |
| 6.5 | Golden Retriever | FS | Distal radius | Osteoblastic | I | 4.5 | 1 × 10\hat{ }9 | 430+ |
| 10 | Greyhound | MC | Distal femur | Osteoblastic | II | 2 | 1 × 10\hat{ }9 | 276 |
| 5.5 | Labrador | MC | Distal femur | Osteoblastic | I | 9 | 1 × 10\hat{ }9 | 312+ |
| 9 | Golden Retriever | FS | Distal femur | Osteoblastic | I | 6 | 1 × 10\hat{ }9 | 336+ |

Group 4

| | | | | | | | | |
|---|---|---|---|---|---|---|---|---|
| 6.6 | Great Dane | MC | Distal radius | Osteoblastic | II | 7.5 | 3 × 10\hat{ }9 | 259 |
| 7 | Mixbreed | MC | Proximal humerus | Osteoblastic | II | 9 | 3 × 10\hat{ }9 | 345+ |
| 6.5 | Rottweiler | FS | Proximal humerus | Osteoblastic | II | 6 | 3 × 10\hat{ }9 | 332+ |

TABLE 5
Signalment and tumor characteristics of dogs with pre-existing metastatic disease
treated on a compassionate care basis

| | | | | | | | | |
|---|---|---|---|---|---|---|---|---|
| | | | | | | | | OVERALL |
| | | | | | | HER2 | | SURVIVAL |
| AGE | BREED | SEX | TUMOR LOCATION | SUBTYPE | GRADE | SCORE | DOSE | (days) |

Vaccine group with mestastic disease

| | | | | | | | | |
|---|---|---|---|---|---|---|---|---|
| 5 | Neopolitan Mastiff | MC | Distal radius | Fibroblastic | I | 7.50 | 1 × 10\hat{ }9 | 233 |
| 6.5 | Great Dane | FS | Distal radius | | | 6 | 1 × 10\hat{ }9 | 256 |
| 2 | Labrador | F | Proximal fibula | Osteoblastic | III | 7.5 | 1 × 10\hat{ }9 | 153 |
| 6.5 | Bernese Mountain Dog | FS | Distal ulnar | Osteoblastic | III | 8.25 | 3 × 10\hat{ }9 | 336 |
| 7 | Rottweiler | MC | Distal radius | Osteoblastic | II | 4.00 | 3 × 10\hat{ }9 | 231 |

### Results

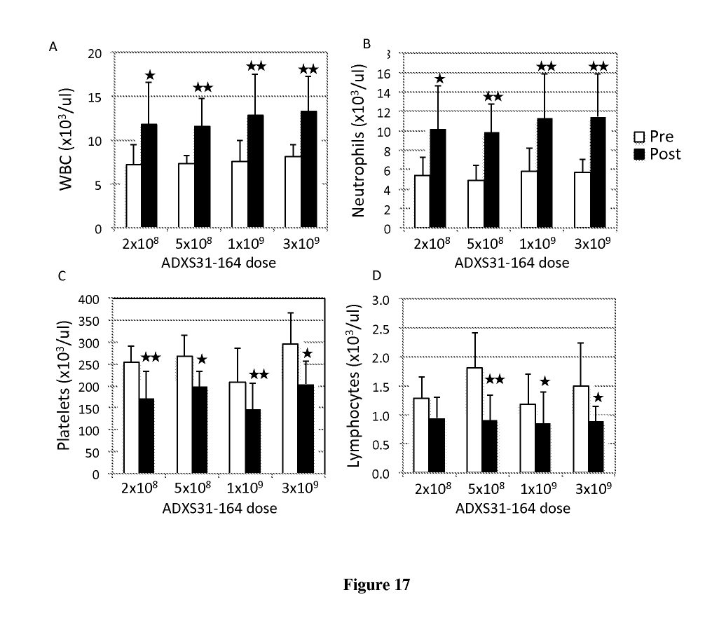

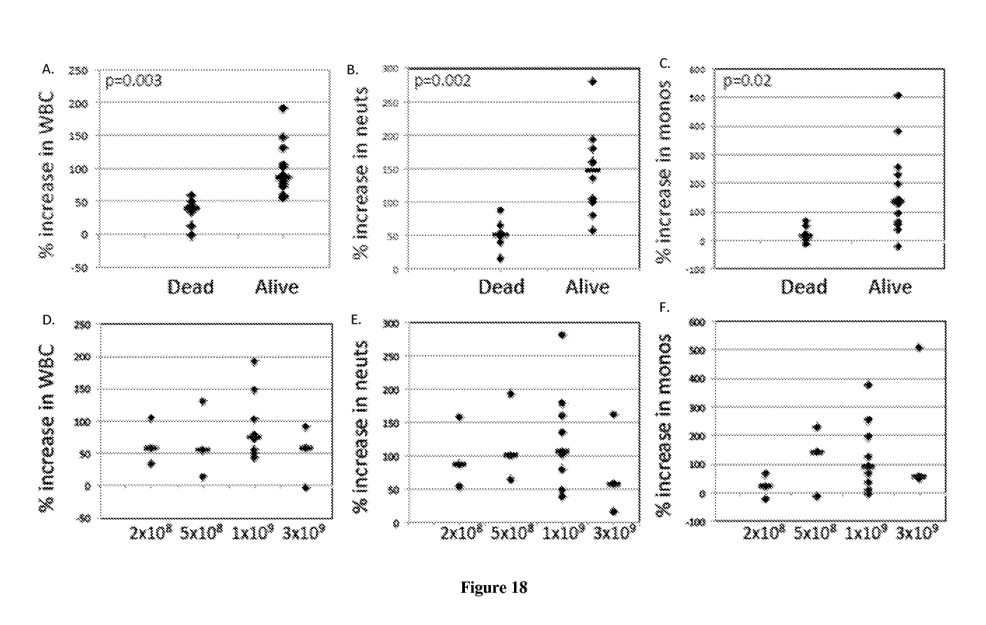

Safety and Toxicity=Safety was evaluated for all 23 vaccinated dogs. All dogs tolerated ADXS31-164 administration well with only transient, low grade toxicities observed on the day of vaccination (Table 6). A statistically significant increase in body temperature occurred 4 hours after ADXS31-164 administration in all groups irrespective of dose (FIG. 9A). Hypotension was not observed at any time point or at any dose (FIG. 9B). 8/18 dogs (without pre-existing metastatic disease) and 3/5 dogs (with pre-existing metastatic disease) developed fevers of >103° F. within 4 hours of vaccination and were given intravenous fluids at that time. Three dogs received a single dose of a non-steroid anti-inflammatory drug to reduce body temperature. In all cases, fevers resolved without further intervention. Transient lethargy, nausea and vomiting that did not require therapeutic intervention occurred within 4 hours of vaccination regardless of dose. In two dogs transient single or bigeminal ventricular premature contractions were identified shortly after vaccination. One dog with pre-existing metastatic disease developed ventricular tachycardia within 2 hours of vaccination. Treatment with lidocaine, procainamide, sotalol and corticosteroids had little effect however, the arrhythmia resolved within 72 hours. Transient, but statistically significant increases in white blood cell and neutrophil counts occurred 24 hours after ADXS31-164 and were accompanied by a transient decrease in platelets and lymphocytes (FIG. 17). Although there was no correlation between ADXS31-164 dose and magnitude of hematological change, there was a significant difference in the magnitude of white blood cell, neutrophil and monocyte responses between dogs that survived and those that died (FIG. 18A-F). Mild, transient increases in the serum concentrations of liver enzymes occurred in approximately half of the dogs, consistent with mild inflammation caused by the hepatotropic Listeria (Table 6). All changes identified in the peripheral blood were asymptomatic and resolved within one week of ADXS31-164 administration. No significant changes in renal function were documented in any dog. 19/23 dogs had blood cultures performed 24 hours after ADXS31-164 administration and all were negative, consistent with rapid clearance of the highly attenuated LmddA strain.

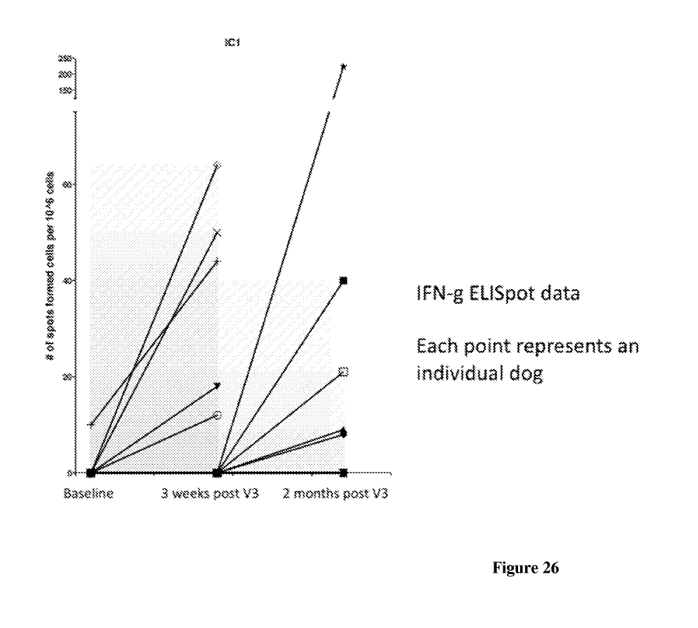

Given that HER2/neu targeted monoclonal antibodies cause cardio toxicity we evaluated biomarkers of cardiac damage and echocardiographic measures of dysfunction including cardiac troponin I, fractional shortening (%), LVIDd and LVIDs at baseline, prior to each vaccination and every 2 months thereafter. No significant, sustained changes in cardiac troponin I, fractional shortening, LVIDd or LVIDs were identified in any of the vaccinated dogs (FIG. 26A-D). One dog in Group 3 showed a stepwise increase in serum cardiac troponin I at the time of each vaccination however, this was not accompanied by echocardiographic signs of dysfunction. Values returned to baseline following the last vaccination and were not elevated on repeat assessments.

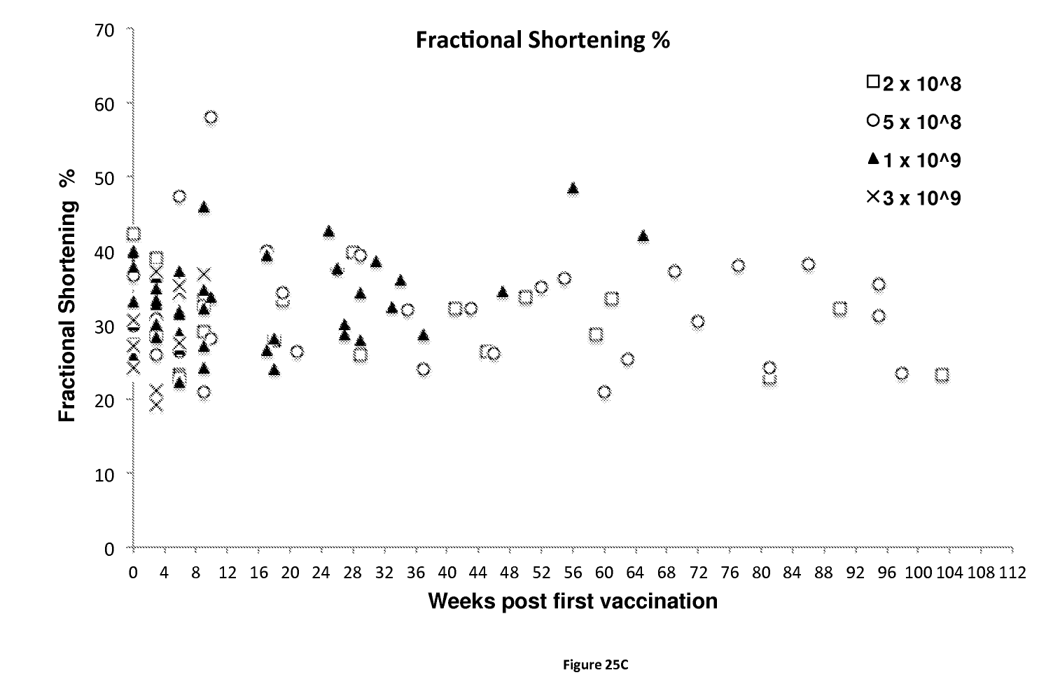

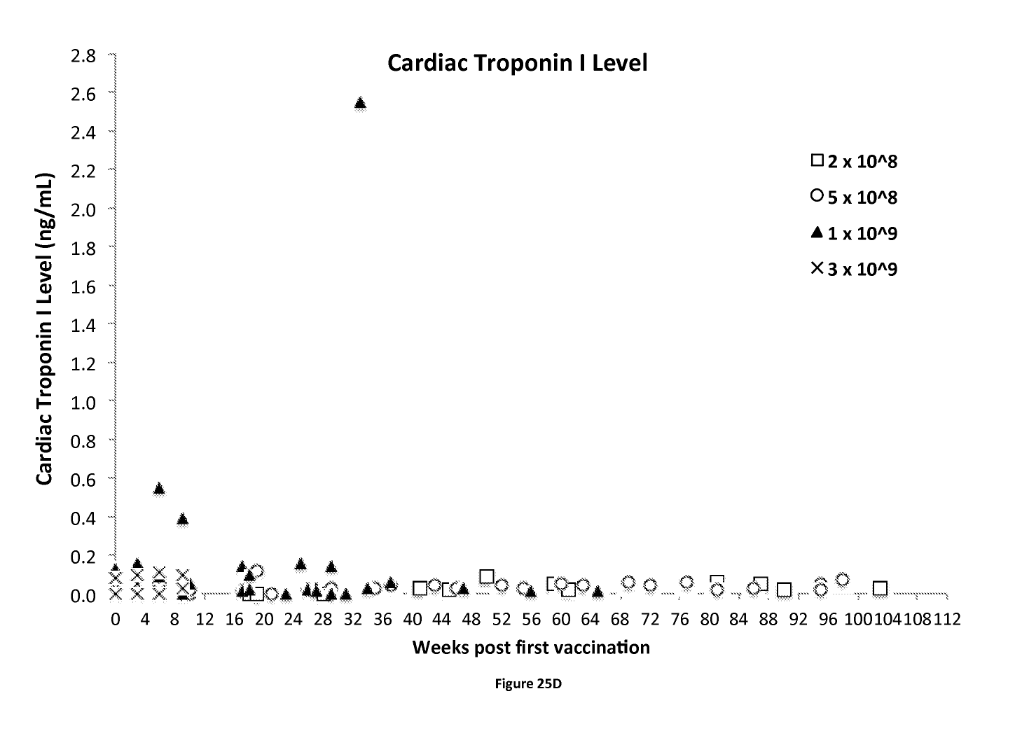

Throughout the clinical trial cardiac troponin I levels were measured along with fractional shortening, Left Ventricular Internal Diameter in systole (LVIDs) and LVID in diastole (LVIDd) as shown in FIG. 25 (A-D), there was no evidence of long or short-term cardio toxicity following administration of ADXS31-164.

Table 6 below presents data showing minimal treatment related adverse events were reported during the clinical trial.

TABLE 6
Treatment Related Adverse Events occurring at or within
48 hours of ADXS31-164 vaccination.
Number of Dogs with Treatment Related Adverse Events

| | ADXS31-164 dose | | | | |
|---|---|---|---|---|---|
| | | | | 3 × | |
| | 2 × 10⁸ | 5 × 10⁸ | 1 × 10⁹ | 10⁹ | Total |
| Number of dogs recruited | 3 | 3 | 11 | 6 | 23 |

General Disorders

| | | | | | | |
|---|---|---|---|---|---|---|
| Pyrexia (T>103) | Grade 1 | 2 | 1 | 5 | 5 | 13 |
| Fatigue | Grade 1 | 1 | 0 | 7 | 2 | 10 |
| Nausea | Grade 1 | 1 | 2 | 10 | 2 | 15 |
| | Grade 2 | 1 | 0 | 0 | 0 | 1 |
| Vomiting | Grade 1 | 1 | 2 | 9 | 3 | 15 |
| | Grade 2 | 2 | 0 | 0 | 3 | 5 |

Cardiovascular abnormalities

| | | | | | | |
|---|---|---|---|---|---|---|
| Arrhythmias | Grade 1 | 0 | 1 | 0 | 0 | 1 |
| | Grade 2 | 0 | 0 | 0 | 1 | 1 |
| Tachycardia | Grade 1 | 0 | 0 | 2 | 1 | 3 |
| | Grade 2 | 0 | 0 | 0 | 1 | 1 |
| Hyoptension | | 0 | 0 | 0 | 0 | 0 |
| Hypertension | Grade 1 | 2 | 3 | 8 | 5 | 18 |

Hematological parameters

| | | | | | | |
|---|---|---|---|---|---|---|
| Thrombocytopenia | Grade 1 | 2 | 2 | 6 | 3 | 13 |
| | Grade 2 | 0 | 0 | 2 | 1 | 3 |

Biochemical parameters (Increased)

| | | | | | | |
|---|---|---|---|---|---|---|
| γ-GT | Grade 1 | 0 | 2 | 1 | 0 | 3 |
| ALKP | Grade 1 | 0 | 1 | 6 | 1 | 8 |
| | Grade 2 | 0 | 0 | 0 | 1 | 1 |
| | Grade 3 | 1 | 0 | 0 | 0 | 1 |
| ALT | Grade 1 | 1 | 1 | 3 | 0 | 5 |
| | Grade 2 | 0 | 0 | 0 | 1 | 1 |
| | Grade 3 | 1 | 0 | 0 | 0 | 1 |
| AST | Grade 1 | 1 | 1 | 4 | 2 | 8 |
| | Grade 2 | 0 | 0 | 2 | 0 | 2 |
| | Grade 3 | 0 | 0 | 1 | 0 | 1 |
| BUN | | 0 | 0 | 0 | 0 | 0 |
| CREA | | 0 | 0 | 0 | 0 | 0 |
| Cardiac Troponin I | Grade 1 | 0 | 0 | 1 | 1 | 2 |

Conclusion: ADSX31-164 toxicities were low grade and transient.

Immune Response to ADXS31-164

The results presented in FIG. 18 demonstrate that an early immune response to ADXS31-164 in dogs receiving the vaccines predicted survival of the dogs. FIG. 18 shows that ADXS31-164 induced increases in WBC, neutrophil and monocytes counts, which correlated with survival and were accompanied by a transient decrease in platelets and lymphocytes (FIG. 17).

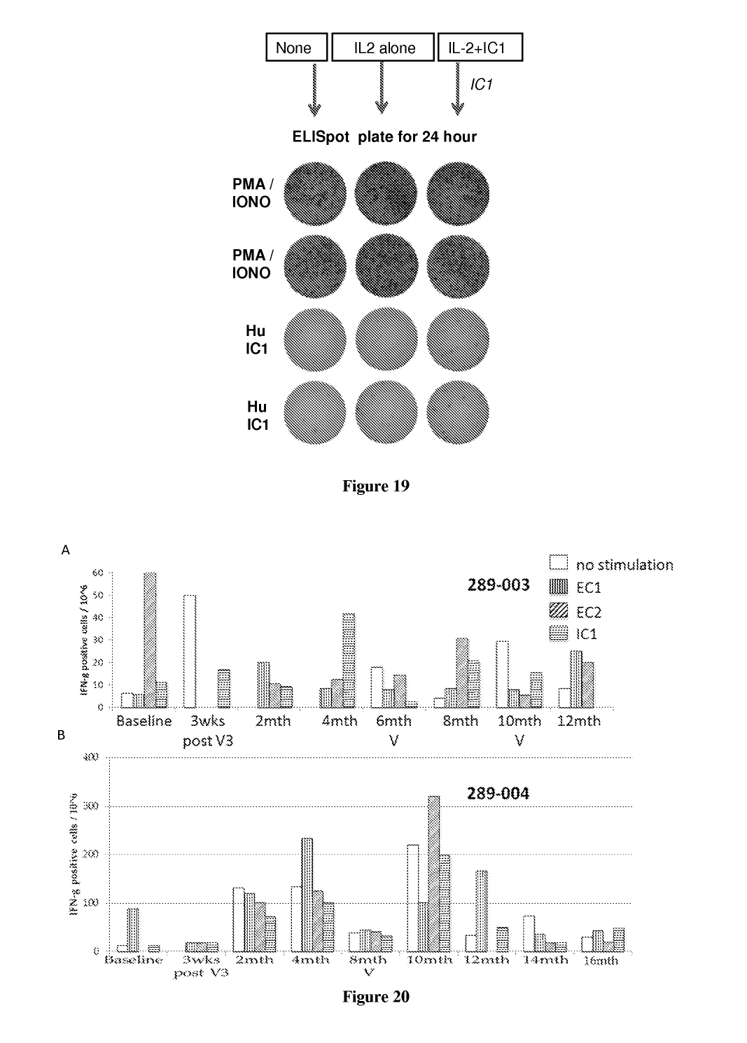

The ability of ADXS31-164 to induce and maintain an immune response, and in particular to induce HER2/Neu specific T cell immunity was assessed during the clinical trial. In order to evaluate the immune response and to determine if a HER2/Neu specific T cell response was induced by ADXS31-164, HER2/Neu specific T cell numbers were assessed by IFN-γ ELISpot. Samples were taken at baseline (3 weeks post carboplatin), at every vaccination and every 2 months thereafter. FIG. 19 shows the results of the ELISpot assay.

HER2/neu Specific Immune Responses. Immunological responses against the human EC1, EC2 and IC1 domains of HER2/neu (sharing 89%, 93% and 98% identity with canine HER2/neu respectively) were detected at baseline in 4/18, 6/18 and 1/18 dogs respectively. Induced IFN-γ responses against one or more of the HER2/neu domains were detected in 7 dogs 3 weeks after the third ADXS31-164 vaccination (Table 7). Five of these dogs developed immune responses against the highly conserved IC1 domain. Five additional dogs developed IFN-γ responses against the IC1 domain 2 months later. Three additional dogs developed IFN-γ responses against either EC2 alone, EC2 and IC1 or EC1, EC2 and EC3 at the time of relapse (dogs 001, 002 and 017). 3 dogs that developed immunological responses against HER2/neu during their initial vaccination series were evaluated by IFN-γ ELISpot over 15 to 17 months. HER2/Neu specific IFN-γ responses were not maintained however, the dogs remained free of metastatic disease during this time. 10 dogs received additional booster vaccinations, of the 6 evaluable, 2 dogs had detectable increases in HER2/neu specific IFN-γ responses 2 months after booster vaccination. Of the 8 dogs that relapsed, 5 had no increase in HER2/neu specific IFN-γ responses 3 weeks after ADXS31-164.

TABLE 7

| | | | | 3 WEEKS POST | | | 2 MONTH | | | 4 MONTH | | | | |
|---|---|---|---|---|---|---|---|---|---|---|---|---|---|---|
| | BASELINE | | | ADXS31-164 | | | RE-CHECK | | | RE-CHECK | | | TIME TO | OVERALL |
| DOG | EC1 | EC2 | IC1 | EC1 | EC2 | IC1 | EC1 | EC2 | IC1 | EC1 | EC2 | IC1 | RELAPSE | SURVIVAL |

Group 1

| | | | | | | | | | | | | | | |
|---|---|---|---|---|---|---|---|---|---|---|---|---|---|---|
| 001 | − | − | − | − | − | − | − | − | − | −^{R} | +^{R} | −^{R} | 350^{B} | 738 |
| 002 | − | + | − | − | − | − | +^{R} | +^{R} | +^{R} | | DEAD | | 185^{L} | 267 |
| 003 | − | + | − | − | − | − | + | + | + | + | + | + | | 1000+ |

Group 2

| | | | | | | | | | | | | | | |
|---|---|---|---|---|---|---|---|---|---|---|---|---|---|---|
| 004 | + | − | − | + | + | + | − | − | − | − | − | − | | 966+ |
| 007 | + | + | − | + | − | − | + | + | + | ND | ND | ND | 869^{B} | 948+ |
| 008 | − | − | − | + | + | + | ND | ND | ND | ND | ND | ND | 318^{B}* | 346 |

Group 3

| | | | | | | | | | | | | | | |
|---|---|---|---|---|---|---|---|---|---|---|---|---|---|---|
| 013 | − | − | − | − | − | − | − | − | + | − | − | + | | 767+ |
| 017 | − | − | − | − | − | − | − | − | − | +^{R} | +^{R} | +^{R} | 322^{B} | 444 |
| 024 | + | − | − | + | − | − | + | − | − | − | + | + | | 511+ |
| 025 | − | − | − | + | + | + | + | − | − | − | − | − | | 461+ |
| 026 | − | + | − | + | + | − | − | − | − | − | + | + | | 462+ |
| 027 | − | − | − | − | − | − | − | + | + | − | − | − | | 453+ |
| 036 | − | − | − | +^{R} | −^{R} | +^{R} | | DEAD | | | | | 251^{L} | 276 |
| 038 | − | − | − | − | − | − | − | + | − | ND | ND | ND | | 335+ |
| 039 | − | − | − | − | + | − | + | + | + | −^{R} | −^{R} | +^{R} | 315^{L} | 359+ |

Group 4

| | | | | | | | | | | | | | | |
|---|---|---|---|---|---|---|---|---|---|---|---|---|---|---|
| 030 | + | + | + | − | + | + | ND | ND | ND | | DEAD | | 226^{L}* | 259 |
| 037 | − | − | − | +^{R} | +^{R} | +^{R} | ND | ND | ND | ND | ND | ND | 190^{L} | 368+ |
| 040 | − | − | − | − | − | − | − | − | − | ND | ND | ND | | 355+ |

Booster vaccinations. Ten of the 18 dogs without metastatic disease at enrollment were administered a single booster vaccine between 5 and 10 months after the initial vaccine series. Four of these dogs received additional booster vaccines given between 4 and 15 months after the first booster vaccine. Similar low grade, transient side effects were noted at the time of booster vaccination as with the initial vaccination series.

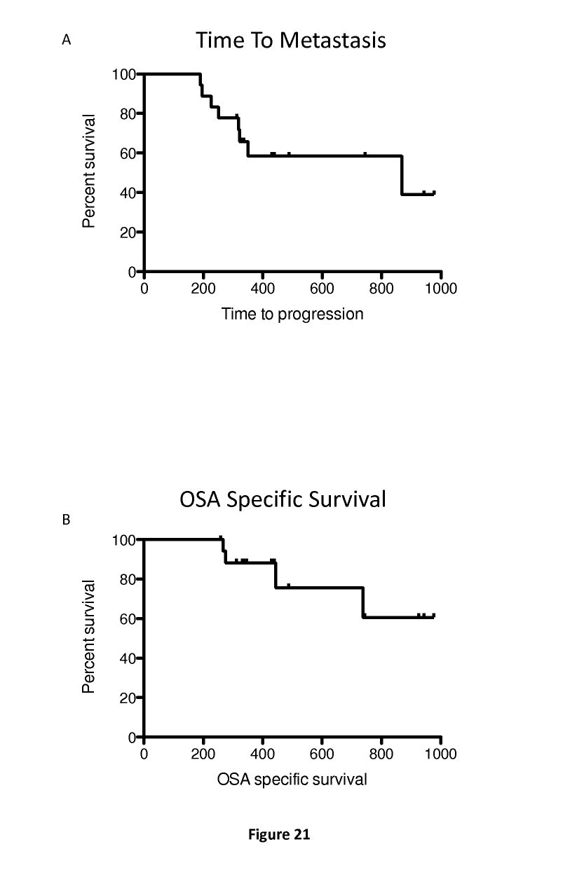

FIGS. 20(A and B) show that repeat booster vaccinations also stimulated HER2 specific immunity. Repeat booster vaccinations were administered at 6 and 10 months for animal 289-003, and at 8 months for animal 289-004. Clinical Outcomes. 8/18 dogs in the vaccinated group relapsed, 4 with pulmonary metastatic disease and 4 with bone metastases. Two dogs with bone metastases progressed to pulmonary metastases. One dog with a bone lesion in her sacrum died from aspiration pneumonia and one dog with a solitary pulmonary nodule died of nephroblastoma however, necropsy specimens from bone and lung lesions respectively were not available for histopathological confirmation of metastatic osteosarcoma. These two dogs were censored from OSA specific survival analysis. Dogs that relapsed received different rescue chemo- and radiation therapies at the discretion of the primary clinician. The 4 dogs with bone metastases were treated with analgesics only (1 dog), palliative radiation alone (1 dog) or in combination with chemotherapy (2 dogs). Two dogs received Adriamycin and 1 dog received palladia for the treatment of pulmonary metastatic disease. Median OSA specific survival for vaccinated dogs has not yet been reached. Kaplan-Meier survival curves for TTM and OSA Specific Survival are shown in FIG. 21. Overall survival rates at 1 and 2 years for vaccinated dogs are 71.4% and 57% respectively. Of the 12 dogs that developed HER2/neu specific IFN-γ responses within 2 months of vaccination, 9 are still alive (3 dogs>900 days, 1 dog>700 days, 3 dogs>400 days and 2 dogs>300 days and 7 remain tumor free to date (Table 7)). The results presented in FIG. 24 demonstrate that ADXS31-164 breaks the tolerance to HER2/Neu. This may be significant for the treatment of OSA as well as other HER2/Neu tumors and/or cancers.

Necropsy findings. 6/18 dogs died during the study period and necropsies were performed on 4 of these dogs. Three dogs were found to have multifocal grade II and III metastatic osteosarcoma involving the lungs (3 dogs), bone (2 dogs), mediastinum (1 dog) and kidney (1 dog). One dog, euthanized on account of a large progressive renal mass was found to have nephroblastoma. This dog also had a single pulmonary nodule but this was unfortunately not evaluated by histopathology.

Survival, Prolonged Survival, Tumor Progression following Administration of ADXS31-164

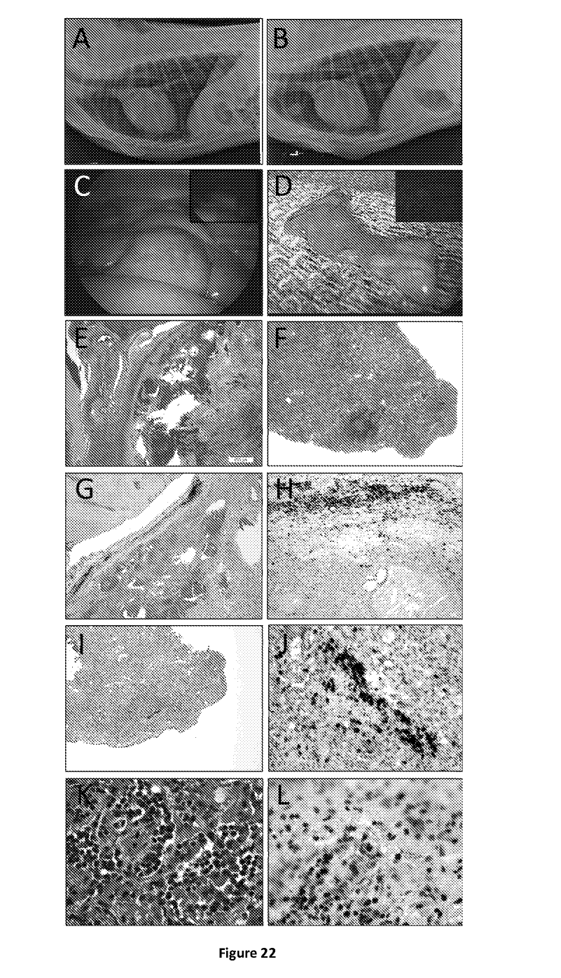

Three dogs with multiple metastatic pulmonary nodules at screening and treated on a compassionate care basis received one vaccine each before disease progression and removal from the study. The two dogs presenting with solitary metastatic pulmonary nodules at the time of screening received all three vaccines (see Table 5 for signalment and tumor characteristics). Progressive pulmonary metastatic disease occurred in one of these dogs despite vaccination. No additional pulmonary lesions developed in the second dog despite the pre-exisiting pulmonary nodule doubling in size every 3 weeks (FIGS. 22A and B). CT scan one week after the last vaccination, confirmed the absence of additional metastatic lesions and the dog underwent metastatectomy. Prior to surgery, the dye indocyanine green (ICG), used to detect tumor margins and areas of inflammation, was administered intravenously and at surgery, fluorescence under near infra-red light was seen in the pulmonary nodule and several other areas of healthy appearing pulmonary parenchyma near the solitary nodule (FIG. 22C and D). Histopathology of the pulmonary nodule revealed metastatic OSA with large areas of hemorrhage and necrosis, surrounded by a thick fibrous capsule (FIG. 22E). IHC showed an accumulation of CD3⁺ T cells around the fibrous capsule with very few T cells within the nodule itself (FIGS. 22G and H). Other areas identified by near infra-red fluorescence showed focal areas of T cell infiltrates (FIGS. 22F, 22I and 22J). T cells were seen surrounding abnormally large, vimentin positive cells with prominent mitotic figures (FIG. 22K and 22L). These findings suggest that single metastatic sarcoma cells may be effectively targeted by tumor specific T cells within the lung and provide a possible mechanism by which ADXS31-164 prevents metastatic pulmonary disease. The dog recovered well from surgery and remained free of pulmonary metastatic disease for 5 months before developing widespread aggressive, HER2/neu+ metastatic disease in the subcutaneous tissue (osteoblastic, grade II and chondroblastic, grade III), mediastinum (osteoblastic, grade II) and diaphragm (osteoblastic, grade III). Results show that despite induction of HER2/neu specific T cell responses, off-tumor side effects were not identified, hence induction of HER2/neu specific T cells is responsible for elimination of HER2/neu positive metastatic cells and long term protection from disease recurrence. This is supported by the timing of HER2/Neu-specific T cell expansion which in 5 dogs occurred approximately 8 months post diagnosis, when many dogs will develop metastatic disease and by the histopathological findings of focal T cell responses within the pulmonary parenchyma of one dog following vaccination and metastatectomy.

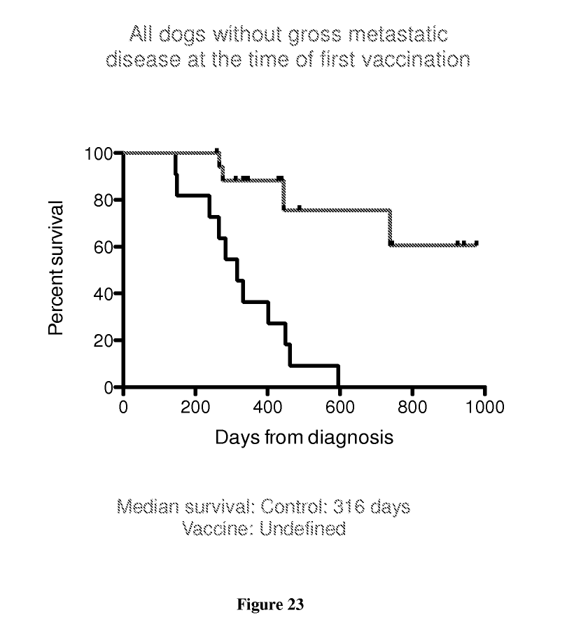

The results presented in FIG. 22 and FIG. 23 demonstrate that administration of ADXS31-164 delays and/or prevents metastatic disease and prolongs the overall survival in dogs with spontaneous HER2+ osteosarcoma. As can be seen in both figures, dogs receiving vaccine had significantly extended survival time, while the median survival for those dogs receiving vaccine has not yet been reached.

While our study demonstrates the effectiveness of this approach in preventing metastatic disease, vaccination with ADXS31-164 was unable to induce regression of pre-existing gross, pulmonary metastatic disease in 5 dogs treated on a compassionate care basis. In one dog this appeared to be associated with a failure of T cells to penetrate the fibrous capsule surrounding the metastatic lesion or for those cells to survive within the established tumor microenvironment (FIG. 22C). However, in the same dog, focal areas of T cell infiltrates surrounding large, actively dividing mesenchymal cells, purported to be metastatic OSA cells were identified in grossly normal lung parenchyma and unexpectedly, following metastatectomy this dog did not develop further pulmonary metastatic disease. Taken together, these data suggest that ADXS31-164 prevents pulmonary metastatic disease through its ability to induce potent innate immune responses that may sensitize metastatic OSA cells to FAS/FASL mediated apoptosis and adaptive immune responses in the form of HER2/Neu specific T cells that eliminate micrometastatic pulmonary disease.

### CONCLUSIONS

At the time of filing this application 12/18 dogs have not developed pulmonary metastatic disease, demonstrating that ADXS31-164 prevents metastatic disease in a subject suffering from spontaneous HER2+ osteosarcoma when administered in the setting of minimal residual disease. Vaccinated dogs showed a statistically significant increase in overall survival compared to a historical HER2/Neu+ control group. Median survival in the HER2/Neu+ control dogs (n=11) was 316 days (p=0.032) wherein the median survival in ADSX31-164 treated dogs has not been reached. Further, the results indicate that ADXS31-164 breaks peripheral tolerance to the highly conserved IC1 domain of HER2/Neu (FIG. 26). The magnitude of the increase in leucocytes within 24 hours of ADXS31-164 administration (FIG. 18) correlates with survival, suggesting that outcome depends in part upon the ability of the dog's immune system to respond to the vaccine Importantly, this study showed that administration of up to 3×10⁹ CFU of ADXS31-164 to dogs with spontaneous OSA is safe and causes only transient, low grade side effects at the time of administration. Moreover, prevention of pulmonary metastatic disease maybe in part associated with CD3+ T cell mediated elimination of microscopic metastatic disease in the lung. This work has important implications for pediatric OSA and other human cancers that express HER2/Neu.

Moreover, here we show that administration of ADXS31-164 in doses up to 3.3×10̂9 CFU are safe in the dog and despite inducing HER2/neu specific immunity, do not lead to short or long term cardio toxicity. On target, off tumor side effects including cardio toxicity has been associated with the administration of large numbers of HER2/neu specific T cells or when trastuzumab has been used concurrently with anthracyclines. We employed a standard chemotherapy protocol without doxorubicin to reduce any potential risk of cardio toxicity.

Our study demonstrates that ADXS31-164 can prevent pulmonary metastatic disease in dogs with OSA. These results demonstrate safety and unprecedented survival times in dogs with OSA and pave the way to investigate the ability of ADXS31-164 to prevent metastatic disease in patients with HER2/neu expressing tumors including pediatric osteosarcoma and mammary carcinoma.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating a HER2/neu-expressing tumor growth or cancer in a subject, the method comprising the step of administering a composition comprising a recombinant attenuated Listeria comprising nucleic acid encoding a recombinant polypeptide, wherein said recombinant polypeptide comprises a HER2/neu chimeric antigen fused to an additional polypeptide, wherein said nucleic acid molecule comprises a first open reading frame encoding said recombinant polypeptide, wherein said nucleic acid molecule further comprises a second open reading frame encoding a metabolic enzyme, and wherein said metabolic enzyme complements an endogenous gene that is mutated in the chromosome of said recombinant Listeria strain.

2. The method of claim 1, wherein said composition comprises a Listeria dose of about 3.3×10⁹ Listeria.

3. The method of claim 1, wherein said subject is a human or a canine subject.

4. The method of claim 3, wherein said human subject is a child, an adolescent or an adult.

5. The method of claim 1, wherein administering said fusion polypeptide to said subject prevents escape mutations within said tumor.

6. The method of claim 1, wherein said HER2/neu chimeric antigen is a human chimeric HER2/neu comprising at least 5, 9, 13, 14, or 17 of the mapped human MHC-class I epitopes.

7. The method of claim 1, wherein said chimeric HER2/neu is a chimeric canine HER2/neu.

8. The method of claim 1, wherein said nucleic acid molecule is integrated into the Listeria genome.

9. The method of claim 1, wherein said nucleic acid molecule is in a plasmid in said recombinant Listeria vaccine strain and wherein said plasmid is stably maintained in said recombinant Listeria vaccine strain in the absence of antibiotic selection.

10. The method of claim 1, wherein said recombinant Listeria comprises a mutation in the actA virulence gene.

11. The method of claim 1, wherein said additional polypeptide is selected from the group consisting of: a) non-hemolytic LLO protein or N-terminal fragment, b) a PEST sequence, or c) an ActA fragment.

12. The method of claim 1, wherein said metabolic enzyme encoded by said second open reading frame is an alanine racemase enzyme or a D-amino acid transferase enzyme.

13. The method of claim 1, further comprising an independent adjuvant.

14. The method of claim 12, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

15. The method of claim 1, wherein said tumor is a HER2/neu positive tumor and wherein said cancer is a HER2/neu-expressing cancer.

16. The method of claim 1, wherein said cancer is osteosarcoma, ovarian cancer, gastric cancer, central nervous system (CNS) cancer, or Ewing's sarcoma (ES).

17. The method of claim 16, wherein said osteosarcoma cancer is a canine osteosarcoma.

18. The method of claim 16, wherein said osteosarcoma is a pediatric osteosarcoma.

19. A method of eliciting an enhanced immune response against a HER2/neu-expressing tumor growth or cancer in a subject, the method comprising the step of administering a composition comprising a recombinant attenuated Listeria strain comprising a nucleic acid encoding a recombinant polypeptide, wherein said fusion polypeptide comprises a HER2/neu chimeric antigen fused to an additional polypeptide, wherein said nucleic acid molecule comprises a first open reading frame encoding said recombinant polypeptide, wherein said nucleic acid molecule further comprises a second open reading frame encoding a metabolic enzyme, and wherein said metabolic enzyme complements an endogenous gene that is mutated in the chromosome of said recombinant Listeria strain.

20. The method of claim 1, wherein said composition comprises a Listeria dose of about 3.3×10⁹ Listeria.

21. The method of claim 19, wherein said subject is a human or a canine subject.

22. The method of claim 21, wherein said human subject is a child, an adolescent or an adult.

23. The method of claim 19, wherein administering said fusion polypeptide to said subject having a Her2/neu-expressing tumor prevents escape mutations within said tumor.

24. The method of claim 19, wherein said HER2/neu chimeric antigen is a human chimeric HER2/neu comprising at least 5, 9, 13, 14, or 17 of the mapped human MHC-class I epitopes.

25. The method of claim 19, wherein said chimeric HER2/neu is a chimeric canine HER2/neu.

26. The method of claim 19, wherein said nucleic acid molecule is integrated into the Listeria genome.

27. The method of claim 19, wherein said nucleic acid molecule is in a plasmid in said recombinant Listeria vaccine strain.

28. The method of claim 19, wherein said plasmid is stably maintained in said recombinant Listeria vaccine strain in the absence of antibiotic selection.

29. The method of claim 19, wherein said recombinant Listeria comprises a mutation in the actA virulence gene.

30. The method of claim 19, wherein said additional polypeptide is selected from the group consisting of: a) non-hemolytic LLO protein or N-terminal fragment, b) a PEST sequence, or c) an ActA fragment.

31. The method of claim 19, wherein said metabolic enzyme encoded by said second open reading frame is an alanine racemase enzyme or a D-amino acid transferase enzyme.

32. The method of claim 19, further comprising an independent adjuvant.

33. The method of claim 32, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

34. The method of claim 19, wherein said tumor is a HER2/neu positive tumor and wherein said cancer is a HER2/neu-expressing cancer.

35. The method of claim 19, wherein said cancer is osteosarcoma, ovarian cancer, gastric cancer, central nervous system (CNS) cancer, or Ewing's sarcoma (ES).

36. The method of claim 35, wherein said osteosarcoma cancer is a canine osteosarcoma.

37. The method of claim 19, wherein said osteosarcoma is a pediatric osteosarcoma.

38. The method of claim 19, wherein said immune response against said HER2/neu-expressing tumor or cancer comprises an immune response to a subdominant epitope of said HER2/neu protein.


SEQUENCE LISTING (74 sequences)

SEQ ID NO: 1 - DNA, 1263 bp, Artificial Sequence
  HER2/neu chimeric protein
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 2 - protein, 420 aa, Artificial Sequence [sequence omitted]

SEQ ID NO: 3 - DNA, 1323 bp, Listeria monocytogenes
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 4 - protein, 441 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 5 - protein, 14 aa, Listeria monocytogenes
KTEEQPSEVNTGPR

SEQ ID NO: 6 - protein, 28 aa, Listeria monocytogenes
KASVTDTSEGDLDSSMQSADESTPQPLK

SEQ ID NO: 7 - protein, 20 aa, Listeria monocytogenes
KNEEVNASDFPPPPTDEELR

SEQ ID NO: 8 - protein, 33 aa, Listeria monocytogenes
RGGIPTSEEFSSLNSGDFTDDENSETTEEEIDR

SEQ ID NO: 9 - protein, 17 aa, Listeria monocytogenes
KQNTASTETTTTNEQPK

SEQ ID NO: 10 - protein, 17 aa, Streptococcus equisimilis
KQNTANTETTTTNEQPK

SEQ ID NO: 11 - protein, 9 aa, Artificial Sequence
  HLA-A2 restricted epitopes located at the extracellular domains of the HER2/neu molecule
HLYQGCQVV

SEQ ID NO: 12 - protein, 9 aa, Artificial Sequence
  HLA-A2 restricted epitopes located at the extracellular domains of the HER2/neu molecule
KIFGSLAFL

SEQ ID NO: 13 - protein, 9 aa, Artificial Sequence
  HLA-A2 restricted epitopes located at the extracellular domains of the HER2/neu molecule
RLLQETELV

SEQ ID NO: 14 - DNA, 55 bp, Artificial Sequence
  Alignment of EC2 (base pairs 975 -1029 of HER2/neu)
ggtcacagctgaggacggaacacagcgttgtgagaaatgcagcaagccctgtgct

SEQ ID NO: 15 - DNA, 60 bp, Artificial Sequence
  Alignment of EC2 (base pairs 975 -1029 of HER2/neu)
cgagtgtgctatggtctgggcatggagcaccttcgaggggcgagggccatcaccagtgac

SEQ ID NO: 16 - DNA, 60 bp, Artificial Sequence
  Alignment of EC2 (base pairs 975 -1029 of HER2/neu)
aatgtccaggagtttgatggctgcaagaagatctttgggagcctggcatttttgccggag

SEQ ID NO: 17 - DNA, 60 bp, Artificial Sequence
  Alignment of EC2 (base pairs 975 -1029 of HER2/neu)
agctttgatggggacccctcctccggcattgctccgctgaggcctgagcagctccaagtg

SEQ ID NO: 18 - DNA, 60 bp, Artificial Sequence
  Alignment of EC2 (base pairs 975 -1029 of HER2/neu)
ttcgaaaccctggaggagatcacaggttacctgtacatctcagcatggccagacagtctc

SEQ ID NO: 19 - DNA, 60 bp, Artificial Sequence
  Alignment of EC2 (base pairs 975 -1029 of HER2/neu)
cgtgacctcagtgtcttccagaaccttcgaatcattcggggacggattctccacgatggc

SEQ ID NO: 20 - DNA, 60 bp, Artificial Sequence
  Alignment of EC2 (base pairs 975 -1029 of HER2/neu)
gcgtactcattgacactgcaaggcctggggatccactcgctggggctgcgctcactgcgg

SEQ ID NO: 21 - DNA, 60 bp, Artificial Sequence
  Alignment of EC2 (base pairs 975 -1029 of HER2/neu)
gagctgggcagtggattggctctgattcaccgcaacgcccatctctgctttgtacacact

SEQ ID NO: 22 - DNA, 60 bp, Artificial Sequence
  Alignment of EC2 (base pairs 975 -1029 of HER2/neu)
gtaccttgggaccagctcttccggaacccacatcaggccctgctccacagtgggaaccgg

SEQ ID NO: 23 - DNA, 60 bp, Artificial Sequence
  Alignment of EC2 (base pairs 975 -1029 of HER2/neu)
ccggaagaggattgtggtctcgagggcttggtctgtaactcactgtgtgcccacgggcac

SEQ ID NO: 24 - DNA, 60 bp, Artificial Sequence
  Alignment of EC2 (base pairs 975 -1029 of HER2/neu)
tgctgggggccagggcccacccagtgtgtcaactgcagtcatttccttcggggccaggag

SEQ ID NO: 25 - DNA, 57 bp, Artificial Sequence
  Alignment of IC1 (base pairs 2114-3042 of HER2/neu)
cgcccagcggagcaatgcccaaccaggctcagatgcggatcctaaaagagacggagc

SEQ ID NO: 26 - DNA, 60 bp, Artificial Sequence
  Alignment of IC1 (base pairs 2114-3042 of HER2/neu)
taaggaaggtgaaggtgcttggatcaggagcttttggcactgtctacaagggcatctgga

SEQ ID NO: 27 - DNA, 60 bp, Artificial Sequence
  Alignment of IC1 (base pairs 2114-3042 of HER2/neu)
tcccagatggggagaatgtgaaaatccccgtggctatcaaggtgttgagagaaaacacat

SEQ ID NO: 28 - DNA, 60 bp, Artificial Sequence
  Alignment of IC1 (base pairs 2114-3042 of HER2/neu)
ctcctaaagccaacaaagaaattctagatgaagcgtatgtgatggctggtgtgggttctc

SEQ ID NO: 29 - DNA, 60 bp, Artificial Sequence
  Alignment of IC1 (base pairs 2114-3042 of HER2/neu)
cgtatgtgtcccgcctcctgggcatctgcctgacatccacagtacagctggtgacacagc

SEQ ID NO: 30 - DNA, 60 bp, Artificial Sequence
  Alignment of IC1 (base pairs 2114-3042 of HER2/neu)
ttatgccctacggctgccttctggaccatgtccgagaacaccgaggtcgcctaggctccc

SEQ ID NO: 31 - protein, 60 aa, Artificial Sequence
  Alignment of IC1 (base pairs 2114-3042 of HER2/neu)
AGGACCTGCTCAACTGGTGTGTTCAGATTGCCAAGGGGATGAGCTACCTGGAGGACGTGC

SEQ ID NO: 32 - protein, 60 aa, Artificial Sequence
  Alignment of IC1 (base pairs 2114-3042 of HER2/neu)
GGCTTGTACACAGGGACCTGGCTGCCCGGAATGTGCTAGTCAAGAGTCCCAACCACGTCA

SEQ ID NO: 33 - DNA, 60 bp, Artificial Sequence
  Alignment of IC1 (base pairs 2114-3042 of HER2/neu)
agattacagatttcgggctggctcggctgctggacattgatgagacagagtaccatgcag

SEQ ID NO: 34 - DNA, 60 bp, Artificial Sequence
  Alignment of IC1 (base pairs 2114-3042 of HER2/neu)
atgggggcaaggtgcccatcaaatggatggcattggaatctattctcagacgccggttca

SEQ ID NO: 35 - DNA, 60 bp, Artificial Sequence
  Alignment of IC1 (base pairs 2114-3042 of HER2/neu)
cccatcagagtgatgtgtggagctatggagtgactgtgtgggagctgatgacttttgggg

SEQ ID NO: 36 - DNA, 59 bp, Artificial Sequence
  Alignment of IC1 (base pairs 2114-3042 of HER2/neu)
ccaaaccttacgatggaatcccagcccgggagatccctgatttgctggagaagggagaa

SEQ ID NO: 37 - DNA, 58 bp, Artificial Sequence
  Alignment of IC1 (base pairs 2114-3042 of HER2/neu)
cgcctacctcagcctccaatctgcaccattgatgtctacatgattatggtcaaatgtt

SEQ ID NO: 38 - DNA, 54 bp, Artificial Sequence
  Alignment of IC1 (base pairs 2114-3042 of HER2/neu)
ggatgattgactctgaatgtcgcccgagattccgggagttggtgtcagaatttt

SEQ ID NO: 39 - DNA, 52 bp, Artificial Sequence
  Alignment of IC1 (base pairs 2114-3042 of HER2/neu)
cacgtatggcgagggacccccagcgttttgtggtcatccagaacgaggactt

SEQ ID NO: 40 - DNA, 60 bp, Artificial Sequence
  Alignment of EC1 (base pairs 399-758 of HER2/neu)
cccaggcagaaccccagaggggctgcgggagctgcagcttcgaagtctcacagagatcct

SEQ ID NO: 41 - DNA, 60 bp, Artificial Sequence
  Alignment of EC1 (base pairs 399-758 of HER2/neu)
gaagggaggagttttgatccgtgggaaccctcagctctgctaccaggacatggttttgtg

SEQ ID NO: 42 - DNA, 60 bp, Artificial Sequence
  Alignment of EC1 (base pairs 399-758 of HER2/neu)
ccgggcctgtccaccttgtgcccccgcctgcaaagacaatcactgttggggtgagagtcc

SEQ ID NO: 43 - DNA, 60 bp, Artificial Sequence
  Alignment of EC1 (base pairs 399-758 of HER2/neu)
ggaagactgtcagatcttgactggcaccatctgtaccagtggttgtgcccggtgcaaggg

SEQ ID NO: 44 - DNA, 60 bp, Artificial Sequence
  Alignment of EC1 (base pairs 399-758 of HER2/neu)
ccggctgcccactgactgctgccatgagcagtgtgccgcaggctgcacgggccccaagca

SEQ ID NO: 45 - DNA, 3716 bp, Rattus norvegicus
ccggaatcgcgggcacccaagtgtgtaccggcacagacatgaagttgcggctccctgccagtcctgagacccacctggacatgctccgccacctgtaccagggctgtcaggtagtgcagggcaacttggagcttacctacgtgcctgccaatgccagcctctcattcctgcaggacatccaggaagttcagggttacatgctcatcgctcacaaccaggtgaagcgcgtcccactgcaaaggctgcgcatcgtgagagggacccagctctttgaggacaagtatgccctggctgtgctagacaaccgagatcctcaggacaatgtcgccgcctccaccccaggcagaaccccagaggggctgcgggagctgcagcttcgaagtctcacagagatcctgaagggaggagttttgatccgtgggaaccctcagctctgctaccaggacatggttttgtggaaggacgtcttccgcaagaataaccaactggctcctgtcgatatagacaccaatcgttcccgggcctgtccaccttgtgcccccgcctgcaaagacaatcactgttggggtgagagtccggaagactgtcagatcttgactggcaccatctgtaccagtggttgtgcccggtgcaagggccggctgcccactgactgctgccatgagcagtgtgccgcaggctgcacgggccccaagcattctgactgcctggcctgcctccacttcaatcatagtggtatctgtgagctgcactgcccagccctcgtcacctacaacacagacacctttgagtccatgcacaaccctgagggtcgctacacctttggtgccagctgcgtgaccacctgcccctacaactacctgtctacggaagtgggatcctgcactctggtgtgtcccccgaataaccaagaggtcacagctgaggacggaacacagcgttgtgagaaatgcagcaagccctgtgctcgagtgtgctatggtctgggcatggagcaccttcgaggggcgagggccatcaccagtgacaatgtccaggagtttgatggctgcaagaagatctttgggagcctggcatttttgccggagagctttgatggggacccctcctccggcattgctccgctgaggcctgagcagctccaagtgttcgaaaccctggaggagatcacaggttacctgtacatctcagcatggccagacagtctccgtgacctcagtgtcttccagaaccttcgaatcattcggggacggattctccacgatggcgcgtactcattgacactgcaaggcctggggatccactcgctggggctgcgctcactgcgggagctgggcagtggattggctctgattcaccgcaacgcccatctctgctttgtacacactgtaccttgggaccagctcttccggaacccacatcaggccctgctccacagtgggaaccggccggaagaggattgtggtctcgagggcttggtctgtaactcactgtgtgcccacgggcactgctgggggccagggcccacccagtgtgtcaactgcagtcatttccttcggggccaggagtgtgtggaggagtgccgagtatggaaggggctcccccgggagtatgtgagtgacaagcgctgtctgccgtgtcaccccgagtgtcagcctcaaaacagctcagagacctgctttggatcggaggctgatcagtgtgcagcctgcgcccactacaaggactcgtcctcctgtgtggctcgctgccccagtggtgtgaaaccggacctctcctacatgcccatctggaagtacccggatgaggagggcatatgccagccgtgccccatcaactgcacccactcctgtgtggatctggatgaacgaggctgcccagcagagcagagagccagcccggtgacattcatcattgcaactgtagtgggcgtcctgctgttcctgatcttagtggtggtcgttggaatcctaatcaaacgaaggagacagaagatccggaagtatacgatgcgtaggctgctgcaggaaactgagttagtggagccgctgacgcccagcggagcaatgcccaaccaggctcagatgcggatcctaaaagagacggagctaaggaaggtgaaggtgcttggatcaggagcttttggcactgtctacaagggcatctggatcccagatggggagaatgtgaaaatccccgtggctatcaaggtgttgagagaaaacacatctcctaaagccaacaaagaaattctagatgaagcgtatgtgatggctggtgtgggttctccgtatgtgtcccgcctcctgggcatctgcctgacatccacagtacagctggtgacacagcttatgccctacggctgccttctggaccatgtccgagaacaccgaggtcgcctaggctcccaggacctgctcaactggtgtgttcagattgccaaggggatgagctacctggaggacgtgcggcttgtacacagggacctggctgcccggaatgtgctagtcaagagtcccaaccacgtcaagattacagatttcgggctggctcggctgctggacattgatgagacagagtaccatgcagatgggggcaaggtgcccatcaaatggatggcattggaatctattctcagacgccggttcacccatcagagtgatgtgtggagctatggagtgactgtgtgggagctgatgacttttggggccaaaccttacgatggaatcccagcccgggagatccctgatttgctggagaagggagaacgcctacctcagcctccaatctgcaccattgatgtctacatgattatggtcaaatgttggatgattgactctgaatgtcgcccgagattccgggagttggtgtcagaattttcacgtatggcgagggacccccagcgttttgtggtcatccagaacgaggacttgggcccatccagccccatggacagtaccttctaccgttcactgctggaagatgatgacatgggtgacctggtagacgctgaagagtatctggtgccccagcagggattcttctccccggaccctaccccaggcactgggagcacagcccatagaaggcaccgcagctcgtccaccaggagtggaggtggtgagctgacactgggcctggagccctcggaagaagggccccccagatctccactggctccctcggaaggggctggctccgatgtgtttgatggtgacctggcaatgggggtaaccaaagggctgcagagcctctctccacatgacctcagccctctacagcggtacagcgaggaccccacattacctctgccccccgagactgatggctatgttgctcccctggcctgcagcccccagcccgagtatgtgaaccaatcagaggttcagcctcagcctcctttaaccccagagggtcctctgcctcctgtccggcctgctggtgctactctagaaagacccaagactctctctcctgggaagaatggggttgtcaaagacgtttttgccttcgggggtgctgtggagaaccctgaatacttagtaccgagagaaggcactgcctctccgccccacccttctcctgccttcagcccagcctttgacaacctctattactgggaccagaactcatcggagcaggggcctccaccaagtaactttgaagggacccccactgcagagaaccctgagtacctaggcctggatgtacctgta

SEQ ID NO: 46 - DNA, 360 bp, Rattus norvegicus
cccaggcagaaccccagaggggctgcgggagctgcagcttcgaagtctcacagagatcctgaagggaggagttttgatccgtgggaaccctcagctctgctaccaggacatggttttgtggaaggacgtcttccgcaagaataaccaactggctcctgtcgatatagacaccaatcgttcccgggcctgtccaccttgtgcccccgcctgcaaagacaatcactgttggggtgagagtccggaagactgtcagatcttgactggcaccatctgtaccagtggttgtgcccggtgcaagggccggctgcccactgactgctgccatgagcagtgtgccgcaggctgcacgggccccaagca

SEQ ID NO: 47 - DNA, 618 bp, Rattus norvegicus
ggtcacagctgaggacggaacacagcgttgtgagaaatgcagcaagccctgtgctcgagtgtgctatggtctgggcatggagcaccttcgaggggcgagggccatcaccagtgacaatgtccaggagtttgatggctgcaagaagatctttgggagcctggcatttttgccggagagctttgatggggacccctcctccggcattgctccgctgaggcctgagcagctccaagtgttcgaaaccctggaggagatcacaggttacctgtacatctcagcatggccagacagtctccgtgacctcagtgtcttccagaaccttcgaatcattcggggacggattctccacgatggcgcgtactcattgacactgcaaggcctggggatccactcgctggggctgcgctcactgcgggagctgggcagtggattggctctgattcaccgcaacgcccatctctgctttgtacacactgtaccttgggaccagctcttccggaacccacatcaggccctgctccacagtgggaaccggccggaagaggattgtggtctcgagggcttggtctgtaactcactgtgtgcccacgggcactgctgggggccagggcccaccca

SEQ ID NO: 48 - DNA, 929 bp, Rattus norvegicus
cgcccagcggagcaatgcccaaccaggctcagatgcggatcctaaaagagacggagctaaggaaggtgaaggtgcttggatcaggagcttttggcactgtctacaagggcatctggatcccagatggggagaatgtgaaaatccccgtggctatcaaggtgttgagagaaaacacatctcctaaagccaacaaagaaattctagatgaagcgtatgtgatggctggtgtgggttctccgtatgtgtcccgcctcctgggcatctgcctgacatccacagtacagctggtgacacagcttatgccctacggctgccttctggaccatgtccgagaacaccgaggtcgcctaggctcccaggacctgctcaactggtgtgttcagattgccaaggggatgagctacctggaggacgtgcggcttgtacacagggacctggctgcccggaatgtgctagtcaagagtcccaaccacgtcaagattacagatttcgggctggctcggctgctggacattgatgagacagagtaccatgcagatgggggcaaggtgcccatcaaatggatggcattggaatctattctcagacgccggttcacccatcagagtgatgtgtggagctatggagtgactgtgtgggagctgatgacttttggggccaaaccttacgatggaatcccagcccgggagatccctgatttgctggagaagggagaacgcctacctcagcctccaatctgcaccattgatgtctacatgattatggtcaaatgttggatgattgactctgaatgtcgcccgagattccgggagttggtgtcagaattttcacgtatggcgagggacccccagcgttttgtggtcatccagaacgaggacttgggcccatccagccccatggacagtaccttctaccgttcactgctggaa

SEQ ID NO: 49 - DNA, 3798 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctga

SEQ ID NO: 50 - DNA, 393 bp, Homo sapiens
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaag

SEQ ID NO: 51 - DNA, 477 bp, Homo sapiens
aatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgaggg

SEQ ID NO: 52 - DNA, 391 bp, Homo sapiens
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagact

SEQ ID NO: 53 - DNA, 7075 bp, Artificial Sequence
  pAdv164 sequence
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 54 - DNA, 921 bp, Homo sapiens
gccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggat

SEQ ID NO: 55 - DNA, 597 bp, Homo sapiens
tacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagag

SEQ ID NO: 56 - DNA, 1209 bp, Homo sapiens
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggct

SEQ ID NO: 57 - DNA, 28 bp, Artificial Sequence
  HER2-Chimera (F)
tgatctcgagacccacctggacatgctc

SEQ ID NO: 58 - DNA, 49 bp, Artificial Sequence
  HerEC1-EC2F (Junction)
ctaccaggacacgattttgtggaagaatatccaggagtttgctggctgc

SEQ ID NO: 59 - DNA, 49 bp, Artificial Sequence
  HerEC1-EC2R (Junction)
gcagccagcaaactcctggatattcttccacaaaatcgtgtcctggtag

SEQ ID NO: 60 - DNA, 50 bp, Artificial Sequence
  HerEC2-ICIF (Junction)
ctgccaccagctgtgcgcccgagggcagcagaagatccggaagtacacga

SEQ ID NO: 61 - DNA, 50 bp, Artificial Sequence
  HerEC2-ICIR (Junction)
tcgtgtacttccggatcttctgctgccctcgggcgcacagctggtggcag

SEQ ID NO: 62 - DNA, 39 bp, Artificial Sequence
  HER2-Chimera (R)
gtggcccgggtctagattagtctaagaggcagccatagg

SEQ ID NO: 63 - DNA, 28 bp, Artificial Sequence
  HER2-EC1(F)
ccgcctcgaggccgcgagcacccaagtg

SEQ ID NO: 64 - DNA, 31 bp, Artificial Sequence
  HER2-EC1 (R)
cgcgactagtttaatcctctgctgtcacctc

SEQ ID NO: 65 - DNA, 28 bp, Artificial Sequence
  HER2-EC2 (F)
ccgcctcgagtacctttctacggacgtg

SEQ ID NO: 66 - DNA, 30 bp, Artificial Sequence
  Her- 2- EC2 (R)
cgcgactagtttactctggccggttggcag

SEQ ID NO: 67 - DNA, 31 bp, Artificial Sequence
  HER2-HER2-IC1(F)
ccgcctcgagcagcagaagatccggaagtac

SEQ ID NO: 68 - DNA, 30 bp, Artificial Sequence
  HER2-IC1 (R)
cgcgactagtttaagccccttcggagggtg

SEQ ID NO: 69 - protein, 131 aa, Homo sapiens
SLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCK

SEQ ID NO: 70 - protein, 131 aa, Canis lupus
SLSFLQDIQEVQGYVLIAHSQVRQIPLQRLRIVRGTQLFEDNYALAVLDNGDPLEGGIPAPGAAPGGLRELQLRSLTEILKGGVLIQRSPQLCHQDTILWKDVFHKNNQLALTLIDTNRSRACPPCSPACK

SEQ ID NO: 71 - protein, 75 aa, Homo sapiens
TAPLQPEQLQVFETLEEITGYLYISAWPDSLPDLSVFQNLQVIRGRILHNGAYSLTLQGLGISWLGLRSLRELGS

SEQ ID NO: 72 - protein, 75 aa, Canis lupus
TAPLQPEQLRVFEALEEITGYLYISAWPDSLPNLSVFQNLRVIRGRVLHDGAYSLTLQGLGISWLGLRSLRELGS

SEQ ID NO: 73 - protein, 131 aa, Homo sapiens
NQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLED

SEQ ID NO: 74 - protein, 131 aa, Canis lupus; identical to SEQ ID NO: 73